(19) United States
(12) Patent Application Publication
(10) Pub. No.: US 2017/0143214 A1
(43) Pub. Date: May 25, 2017

(54) TEMPERATURE MEASUREMENT SYSTEMS, METHODS AND DEVICES

(71) Applicant: Securus Medical Group, Inc., Cleveland, OH (US)

(72) Inventors: John T. Garibotto, Marblehead, MA (US); Jesse N. Jones, IV, Woburn, MA (US); John A. Schanzle, Belmont, MA (US); Steven D. Girouard, Chagrin Falls, OH (US); Frederick M. Moore, Boston, MA (US); Dan E. Hamilton, Mont Vernon, NH (US); Jon B. Taylor, Groton, MA (US)

(73) Assignee: Securus Medical Group, Inc., Cleveland, OH (US)

(21) Appl. No.: 15/309,307

(22) PCT Filed: Jun. 2, 2015
(86) PCT No.: PCT/US2015/033680

§ 371 Date: Nov. 7, 2016

Related U.S. Application Data
(60) Provisional application No. 62/007,677, filed on Jun. 4, 2014.

(51) Int. Cl. A61B 5/01; G01J 5/08; A61B 5/00; G01J 5/00

(52) U.S. Cl. CPC A61B 5/015; G01J 5/0025; G01J 5/0821; A61B 5/4233; A61B 5/0086

(57) ABSTRACT

A system that produces temperature estimations of a tissue surface comprises a base, a probe assembly having a proximal end and a distal end, a fiber assembly extending through the probe assembly, a motion unit at the base constructed and arranged to at least one of rotate at least one fiber relative to the base about the longitudinal axis and translate the at least one fiber relative to the base in a linear direction along the longitudinal axis, a first coupling mechanism coupled to the base, wherein the handle is removably coupled to the first coupling mechanism, and a second coupling mechanism at the motion unit, wherein the probe connector is removably coupled to the second coupling mechanism.

### RELATED APPLICATIONS

This patent application claims the benefit of U.S. Provisional Application Ser. No. 62/007,677 filed Jun. 4, 2014, and is a continuation-in-part (CIP) of International Patent Application Serial Number PCT/US2013/076961, entitled “Temperature Measurement Systems, Method and Devices,” filed Dec. 20, 2013, which in turn claims the benefit of U.S. Provisional Application Ser. No. 61/749,617 filed Jan. 7, 2013, the content of each of which is incorporated by reference in its entirety.

This patent application is related to International Patent Application Serial Number PCT/US2011/061802, entitled “Ablation and Temperature Measurement Devices”, filed Nov. 22, 2011 and U.S. Provisional Application Ser. No. 61/417,416, filed Nov. 27, 2010, and U.S. patent application Ser. No. 12/934,008 filed Sep. 22, 2010, the content of each of which is incorporated by reference in its entirety.

### FIELD

Embodiments relate generally to the field of tissue temperature monitoring, and more particularly, to ablation and temperature measurement devices and systems that monitor tissue temperature during energy delivery.

### BACKGROUND

Numerous medical procedures include the delivery of energy to change the temperature of target tissue, such as to ablate or otherwise treat the tissue. With today's energy delivery systems, it is difficult for an operator of the system, such as a clinician, to treat all of the target tissue while avoiding adversely affecting non-target tissue. In treatment of a cardiac arrhythmia, ablation of heart tissue can often ablate target tissue such as heart wall tissue, while inadvertently ablating esophageal tissue. Similarly, in airway ablation for the treatment of COPD, asthma, tumors and other airway disorders the esophageal tissue may be inadvertently ablated. In tumor ablation procedures, cancerous tissue ablation may also be incomplete or healthy tissue may be damaged.

There is a need for energy delivery and energy monitoring systems which allow a clinician to properly deliver energy to target tissue, while avoiding any destructive energy delivery to non-target tissue.

### SUMMARY

According to a first aspect, a system for producing surface temperature estimations of a tissue surface comprises a first optical assembly constructed and arranged to receive infrared light emitted from multiple tissue surface areas; a fiber comprising a proximal end and a distal end, where the distal end is optically coupled to receive infrared light from the first optical assembly; and a sensor optically coupled to the fiber proximal end, where the sensor is constructed and arranged to produce a signal that correlates to an average temperature of each of the multiple tissue surface areas.

The system can be constructed and arranged to produce surface temperature estimations of a surface of an esophagus.

The system can further comprise a probe comprising the first optical assembly and the fiber. Probe diameter can be less than or equal to 15 F, or less than or equal to 12 F, or less than or equal to 9 F, or less than or equal to 6Fr. In other embodiments, the probe diameter can be greater than 15 F.

The first optical assembly can comprise a surrounding tube. The surrounding tube can comprise a relatively infrared transmissive tube. The surrounding tube can comprise a material selected from the group consisting of: high density polyethylene (HDPE) or low density polyethylene (LDPE); germanium; and combinations of these.

The first optical assembly can comprise an optical element selected from the group consisting of: optical fiber; lens; mirror; filter; prism; amplifier; refractor; splitter; polarizer; aperture; and combinations of these.

The first optical assembly can comprise an optical element constructed and arranged to perform an action on the received infrared light selected from the group consisting of: focus; split; filter; transmit without filtering; amplify; refract; reflect; polarize; and combinations of these.

The first optical assembly can comprise a rigid length less than or equal to 3 cm, or less than or equal to 2 cm, or less than or equal to 1 cm, or less than or equal to 0.5 cm.

The first optical assembly can comprise an optical element comprising a planar surface, an angled surface and a convex surface. The angled surface can comprise an angle of approximately 45°. The convex surface can comprise a convex surface with approximately a 4 mm radius.

The first optical assembly can comprise an optical element with a first surface constructed and arranged to receive infrared light from tissue and a second surface constructed and arranged to direct the received infrared light to the fiber distal end. The second surface can comprise a convex surface. In various embodiments, surfaces may be treated with an anti-reflective coating specific for infrared wavelengths 8-11 um. The first optical assembly can comprise an optical separation distance between the second surface and the fiber distal end. In some embodiments, the fiber can comprise an approximately 400 μm core, the optical separation distance can comprise a distance of approximately 4.5 mm, and the second surface can comprise a convex radius of approximately 3 mm. In this embodiment, the first optical assembly can comprise a focal length of approximately 3.5 mm, and the system can be constructed and arranged to receive infrared light from multiple tissue surface areas comprising an area of approximately 0.4 mm. In some embodiments, the fiber can comprise an approximately 400 μm core, the optical separation distance can comprise a distance of approximately 4.2 mm, and the second surface can comprise a convex radius of approximately 4 mm. In this embodiment, the first optical assembly can comprise a focal length of approximately 7.5 mm, the system can be constructed and arranged to receive infrared light from multiple tissue surface areas comprising an area of approximately 1.0 mm, and the system can comprise spatial resolution criteria correlating to a depth of field of approximately 8 mm.

The first optical assembly can comprise a focal length of less than or equal to 10 mm, or less than or equal to 5 mm, for example, a focal length of approximately 3.2 mm, or approximately 3.5 mm. The first optical assembly can comprise a focal length between 4 mm and 10 mm.

The system can comprise spatial resolution criteria correlating to a depth of field between 0.1 mm and 15 mm, or a depth of field between 0.1 mm and 1.0 mm, for example a depth of field of approximately 0.5 mm. The system can comprise spatial resolution criteria correlating to a depth of field of between 1.5 mm and 10 mm, for example a depth of field of approximately 7 mm.

The first optical assembly can comprise a flange constructed and arranged to geometrically center the fiber.

The multiple tissue surface areas can comprise multiple tissue surfaces, each comprising an area between 0.1 mm² and 20 mm², or an area between 0.5 mm² and 1.5 mm², for example an area of approximately 1 mm².

The multiple tissue surface areas can comprise multiple tissue surfaces, each comprising an equivalent diameter between 0.5 mm and 1.5 mm.

The multiple tissue surface areas can comprise multiple tissue surfaces, each comprising a major axis comprising a length between 0.5 mm and 1.5 mm.

The multiple tissue surface areas can comprise multiple tissue surfaces, each comprising a relatively circular geometry, or a relatively rectangular geometry. The multiple tissue surface areas can comprise multiple relatively flat tissue surfaces, or can comprise multiple peaks and valleys. The multiple tissue surface areas can comprise multiple tubular tissue surface areas, for example a segment of the esophagus.

The fiber can comprise a material selected from the group consisting of: zinc selenide, germanium; germanium oxide, silver halide; chalcogenide; a hollow core fiber material; and combinations of these.

The fiber can comprise a material relatively transmissive to wavelengths between 6 μm and 15 μm, or wavelengths between 8 μm and 11 μm.

The fiber can comprise a bundle of fibers. The bundle of fibers can comprise coherent or non-coherent fibers.

The fiber can comprise at least one anti-reflective coating. For example, the at least one anti-reflective coating can be positioned on at least one of the fiber proximal end or the fiber distal end. The at least one anti-reflective coating can comprise a coating selected from the group consisting of: a broadband anti-reflective coating such as a coating covering a range of 6 μm-15 μm or a range of 8 μm-11 μm; a narrow band anti-reflective coating such as a coating covering a range of 7.5 μm-8 μm or a range of 8 μm-9 μm; a single line anti-reflective coating such as a coating designed to optimally reflect a single wavelength or a very narrow range of wavelengths in the infrared region; and combinations of these.

The fiber can further comprise a core comprising a diameter between 6 μm and 100 μm, or a diameter between 200 μm and 400 μm.

The fiber can further comprise a core and a surrounding cladding. The fiber further can comprise a core and an air envelope surrounding the core.

The fiber further can comprise a twist resisting structure surrounding at least a portion of the fiber between the fiber proximal end and the fiber distal end, for example a structure selected from the group consisting of: coil; braid; and combinations of these. The twist resisting structure can comprise a torque shaft. The torque shaft can comprise multiple layers of wires wound in opposite directions. The torque shaft can comprise four to twelve wires.

The system can be constructed and arranged to perform a manipulation on the fiber selected from the group consisting of: rotating the fiber; translating the fiber; and combinations of these. The fiber can comprise a service loop constructed and arranged to accommodate translation motion.

The fiber can further comprise a sleeve surrounding at least a portion of the fiber where the sleeve can comprise a material constructed and arranged to be non-reactive with at least a portion of the fiber. For example, the fiber can comprise a core, and the sleeve material can be constructed and arranged to be non-reactive with the core.

The sensor can comprise an infrared light detector. The sensor can comprise a sensor selected from the group consisting of: a photoconductor such as a mercury cadmium telluride photodetector or a mercury zinc telluride photodetector, a microbolometer; a pyroelectric detector such as a lithium tantalite detector or triclycine sulfate detector, a thermopile; and combinations of these.

The sensor can comprise a response time less than or equal to 200 milliseconds, or a response time less than or equal to 1 millisecond.

The sensor can comprise a cooling assembly constructed and arranged to cool one or more portions of the sensor. The cooling assembly can comprise a cooling assembly selected from the group consisting of: liquid nitrogen filled dewar; thermoelectric cooler; Stirling cycle cooler; and combinations of these.

The signal can comprise a voltage signal and/or a current signal. The signal can represent a change in received infrared light.

The system can further comprise a shaft, where the fiber is slidingly received by the shaft. The shaft can comprise a rounded tip. The shaft can comprise a material selected from the group consisting of: polyethylene; polyimide; polyurethane; polyether block amide; and combinations of these. The shaft can comprise a braided shaft. The shaft can be constructed and arranged for over-the-wire insertion into a body lumen. The shaft can be constructed and arranged for insertion into a nostril. The shaft can be constructed and arranged to be inserted through anatomy with a radius of curvature less than or equal to 4 inches, or a radius of curvature less than or equal to 2 inches, or a radius of curvature less than or equal to 1 inch.

The system can further comprise a second optical assembly constructed and arranged to receive infrared light from the fiber and direct light onto a receiving surface of the sensor. The second optical assembly can comprise an adjustment assembly constructed and arranged to allow at least two-dimensional positioning of the second optical assembly relative to the sensor.

The second optical assembly can comprise an optical element selected from the group consisting of: optical fiber; lens; mirror; filter; prism; amplifier; refractor; splitter; polarizer; aperture; and combinations of these. The second optical assembly can comprise an optical element constructed and arranged to perform an action on the received infrared light selected from the group consisting of: focus; split; filter; transmit without filtering; amplify; refract; reflect; polarize; and combinations of these.

The system can comprise a cooled housing and, at least a portion of the second optical assembly can be maintained within the cooled housing, for example a Stirling cooled housing.

The second optical assembly comprises a component comprising an anti-reflective surface.

The second optical assembly can comprise a component relatively transmissive of light with a wavelength between 6 μm and 15 μm, or a wavelength between 8 μm and 11 μm.

The second optical assembly can comprise a focusing lens. The focusing lens can be separated from a least a portion of the sensor by a gap, for example an operator adjustable gap.

The second optical assembly can comprise a filter. The filter can be relatively non-transmissive of light with a wavelength below 8 μm and/or relatively non-transmissive of light with a wavelength above 11 μm.

The second optical assembly can comprise a cold aperture.

The second optical assembly can comprise an immersion lens.

The second optical assembly can be constructed and arranged to overfill the receiving surface of the sensor with the infrared light received from the fiber. For example, the second optical assembly can be constructed and arranged to overfill the sensor to perform an action selected from the group consisting of: minimizing infrared light emanating from surfaces other than the fiber proximal end onto the sensor receiving surface; minimizing errors caused by light emanating from the fiber proximal end moving at least one of on or off the receiving surface; and combinations of these. The second optical assembly can be constructed and arranged to underfill the receiving surface of the sensor with the infrared light received from the fiber. For example, the second optical assembly can be constructed and arranged to underfill the sensor to maximize the amount of light received by the receiving surface of the sensor that emanates from the fiber proximal end. The system can be constructed and arranged to allow an operator to adjust the amount of at least one of overfill or underfill.

The second optical assembly can be constructed and arranged to deliver the infrared light received from the fiber in a pattern matching the geometry of the receiving surface of the sensor. In some embodiments, the system can be constructed and arranged to deliver the infrared light received from the fiber in a rectangular pattern to the receiving surface of the sensor, where the receiving surface of the sensor comprises a rectangular pattern. In some embodiments, the system can be constructed and arranged to deliver the infrared light received from the fiber in a circular pattern to the receiving surface of the sensor, where the receiving surface of the sensor comprises a circular pattern. In some embodiments, the system can be constructed and arranged to deliver the infrared light received from the fiber in an elliptical pattern to the receiving surface of the sensor, where the receiving surface of the sensor comprises an elliptical pattern. In some embodiments, the system can be constructed and arranged to deliver the infrared light received from the fiber in a square pattern to the receiving surface of the sensor, where the receiving surface of the sensor comprises a square pattern.

The system can further comprise a rotating assembly. The rotating assembly can be constructed and arranged to rotate the fiber and/or the first optical assembly. The system can further comprise a translating assembly constructed and arranged to translate the fiber. The system can be constructed and arranged to simultaneously rotate and translate the fiber or sequentially rotate and translate the fiber. The rotating assembly can be constructed and arranged to provide a 360° rotation. The rotating assembly can be constructed and arranged to provide a reciprocating rotation less than 360°, such as a reciprocating rotation between 45° and 320°, or a reciprocating motion of less than or equal to 180°, or a reciprocating motion of less than or equal to 90°.

The rotating assembly can comprise a rotational encoder.

The rotating assembly can be constructed and arranged to rotate the fiber at a velocity between 1000 rpm and 15000 rpm, or a velocity between 4000 rpm and 8000 rpm, for example a velocity of approximately 7260 rpm.

The rotating assembly can comprise an adjustment assembly constructed and arranged to allow an operator to adjust the position of at least a portion of the fiber, for example the fiber proximal end. The adjustment assembly can be constructed and arranged to provide at least two dimensions of adjustment.

The system can further comprise a translating assembly constructed and arranged to translate the fiber and/or the sensor. The translating assembly can be constructed and arranged to translate the fiber in a reciprocating motion. The system can further comprise a rotating assembly constructed and arranged to rotate the fiber. The translating assembly can be further constructed and arranged to translate the rotating assembly. The system can be constructed and arranged to simultaneously rotate and translate the fiber, or sequentially rotate and translate the fiber.

The translating assembly can be constructed and arranged to translate the fiber a distance between 5 mm and 100 mm, or a distance between 10 mm and 40 mm, for example a distance of approximately 25 mm.

The translating assembly can comprise a linear encoder. The translating assembly can comprise a yankee screw.

The translating assembly can be constructed and arranged to provide a relatively continuous translation of the fiber. The translating assembly can be constructed and arranged to translate the fiber for a first time period and a second time period, where the first and second time periods are separated by a delay.

The system can further comprise a user interface. The user interface can be constructed and arranged to display a graphical temperature map of the average temperature of each of the multiple tissue surface areas. The user interface can be constructed and arranged to depict temperature differences by varying a graphical parameter selected form the group consisting of: color; hue; contrast; and combinations of these. The user interface can be constructed and arranged to allow an operator to adjust a temperature versus a graphic parameter correlation.

The user interface can be constructed and arranged to display a temperature map of a two dimensional representation of body tissue and/or a three dimensional representation of body tissue.

The user interface can be constructed and arranged to display an alphanumeric table of temperature information.

The user interface can be constructed and arranged to display and continually update a temperature map of the average temperature of each of the multiple tissue surface areas. For example, the user interface can be constructed and arranged to update the temperature map every 0.1 seconds to every 30 seconds, or every 0.2 seconds to every 5 seconds, or every 0.5 seconds to every 2 seconds, for example approximately every 1 second.

The user interface can further comprise a user input component. The user input component can comprise a component selected from the group consisting of: touch screen monitor; a keyboard; a mouse; a joystick; and combinations of these.

The user interface can be constructed and arranged to allow an operator to calibrate the sensor. The user interface can be constructed and arranged to allow an operator to adjust a motion parameter selected form the group consisting of: a rotation parameter such as rotational travel distance and/or rotational speed; a translation parameter such as translation travel distance and/or translational speed; scanning pattern geometry; and combinations of these.

The user interface can be constructed and arranged to display other temperature information, for example at least one of peak temperature information and average temperature information for multiple tissue surfaces.

The system can further comprise a signal processing unit. The signal processing unit can be constructed and arranged to correlate the sensor signal into a table of temperature values correlating to the multiple tissue surface areas. The system can further comprise a video monitor, and the signal processing unit can produce a video signal constructed and arranged to drive the video monitor. The signal processing unit can comprise an algorithm, for example an algorithm constructed and arranged to perform a function selected from the group consisting of: averaging one or more values such as temperature values; finding the peak value of one or more temperature values; comparing peak values of one or more tissue areas; rate of change of tissue temperature; rate of rate of change of tissue temperature; determining an outlier value; and combinations of these. Additionally, the algorithm can be constructed and arranged to determine an area of tissue whose average temperature is higher than other tissue areas measured.

The system can further comprise at least one band, where the first optical assembly can collect infrared light emanating from the at least one band. The at least one band can comprise a proximal band, and the first optical assembly can be constructed and arranged to translate between a proximal position and distal position, and where the proximal band is positioned relative the proximal position. The at least one band can comprise a distal band, and the first optical assembly can be constructed and arranged to translate between a proximal position and distal position, where the distal band is positioned relative the distal position. The at least one band can comprise a distal band and a proximal band, and the first optical assembly can be constructed and arranged to translate between the distal band and the proximal band. The at least one band can comprise a material selected from the group consisting of: a thermally conductive material; aluminum, titanium, gold, copper, steel; and combinations of these. The at least one band can be constructed and arranged to cause the sensor to produce a predetermined signal when the first optical element receives infrared light from the at least one band.

The system can further comprise at least one temperature sensor constructed and arranged to measure a temperature of the at least one band. The at least one temperature sensor can comprise a sensor selected from the group consisting of: thermocouple; thermisters; and combinations of these. The system can be constructed and arranged to calibrate the sensor based on the measured temperature. The system can be constructed and arranged to calibrate the sensor multiple times, where the calibration can be based on the measured temperature. For example, the optical assembly can be constructed and arranged to translate, and the system can be constructed and arranged to calibrate the sensor for every optical assembly translation.

The at least one band can comprise a first band and a second band, and the system can further comprise a second temperature sensor constructed and arranged to measure a temperature of the second band. For example, the optical assembly can be constructed and arranged to translate, and the system can be constructed and arranged to calibrate the sensor two times for every optical assembly translation.

The at least one band can comprise a visualization marker, for example a marker selected from the group consisting of: a radiopaque marker such as a radiopaque marker band; an ultrasonically reflective marker; a visible light marker; a magnetic marker; and combinations of these.

The system can further comprise a positioning member. The positioning member can be constructed and arranged to position the first optical assembly at a distance from the tissue surface. The positioning member can be constructed and arranged to center the first optical assembly in a body lumen, for example in an esophagus.

According to another aspect, a system for producing surface temperature estimations of a tissue surface comprises an elongate probe comprising a first optical assembly constructed and arranged to receive infrared light emitted from multiple tissue surface areas and a fiber comprising a proximal end and a distal end, where the distal end is optically coupled to receive infrared light from the first optical assembly; and a sensor optically coupled to the fiber proximal end, where the sensor is constructed and arranged to produce a signal that correlates to an average temperature of each of the multiple tissue surface areas.

According to another aspect, a system for producing surface temperature estimations of a tissue surface comprises a fiber comprising a proximal end and a distal end, where the fiber is constructed and arranged to allow infrared light to pass therethrough; an optical assembly optically coupled to the distal end of the fiber, where the optical assembly is constructed and arranged to receive infrared light emitted from at least one tissue surface area; and a sensor optically coupled to the fiber proximal end, where the sensor is constructed and arranged to produce a signal based on the infrared light emitted from the at least one tissue surface area, and where the signal correlates to an average temperature of the at least one tissue surface area.

According to another aspect, a method of producing surface temperature estimations of a tissue surface comprises: selecting a system as described herein; deploying at least a portion of the system to a tissue surface of a patient location; and producing surface temperature estimations in the region of the tissue surface.

According to another aspect, a system that produces temperature estimations of a tissue surface, comprises a base; a probe assembly having a proximal end and a distal end, the proximal end of the probe assembly at the base and extending along a longitudinal axis, and including: a handle at the proximal end of the probe assembly; and a probe connector; a fiber assembly extending through the probe assembly, the fiber assembly including at least one fiber constructed and arranged to receive infrared energy from the tissue surface; a motion unit at the base, the motion unit constructed and arranged to at least one of rotate the at least one fiber relative to the base about the longitudinal axis and translate the at least one fiber relative to the base in a linear direction along the longitudinal axis; a first coupling mechanism coupled to the base, wherein the handle is removably coupled to the first coupling mechanism; and a second coupling mechanism at the motion unit, wherein the probe connector is removably coupled to the second coupling mechanism.

The motion unit can comprise a rotary motor having a hollow shaft, wherein the probe connector is positioned in the hollow shaft, and wherein the hollow shaft is driven by the motion unit to rotate the at least one fiber about the longitudinal axis; and a linear motor that translates the at least one fiber and the rotary motor in a linear direction along the longitudinal axis.

The rotary motor assembly and the linear motor can operate independently of each other.

The motion unit can comprise a rotary motor having a hollow shaft, wherein the probe connector is positioned in the hollow shaft, and wherein the hollow shaft is driven by the motion unit to rotate the at least one fiber about the longitudinal axis.

A proximal end of the probe connector can include a conical nose, wherein a proximal end of the at least one fiber is at the conical nose, and wherein a proximal end of the hollow shaft of the rotary motor mates with the conical nose of the probe connector.

The system can further comprise an optical element adjacent the rotary motor, wherein the conical nose is positioned in the hollow shaft such that the at least one fiber is aligned with the optical element along the longitudinal axis.

The conical nose of the probe connector can be conformably positioned in a conical cavity of the hollow shaft of the rotary motor to maintain concentricity between the at least one fiber and the optical element during operation of the system.

When the rotary motor rotates between two positions at a predetermined angle between the two positions, the at least one fiber can rotate at the same predetermined angle and at the same time as the rotary motor.

The second coupling mechanism can include a spring-biased rotary motor coupling at the hollow shaft of the rotary motor, the spring-biased rotary motor coupling having at least one groove, and wherein the probe connector includes at least one engagement pin constructed and arranged to mate with the at least one groove at the hollow shaft of the rotary motor.

The system can further comprise an automatic coupling mechanism that couples the probe connector to the rotary motor by detecting the handle at the first coupling mechanism, and drives a connection interface of the rotary motor to the probe connector for interfacing with the probe connector.

The rotary motor can include a plurality of counterweights coupled to the hollow shaft for providing a centripetal force. The second coupling mechanism can be positioned at the counterweights for coupling to a proximal end of the probe connector.

The second coupling mechanism can comprise a collet and wherein the probe connector comprises a coupling that interfaces with the collet.

The probe connector can comprise at least one slot, the hollow shaft comprises at least one opening that aligns with the at least one slot of the probe connector, and wherein the system further comprises a linkage device that is positioned in the aligned at least one slot and opening to prevent the probe connector from moving axially with respect to the hollow shaft.

The system can further comprise a control device that controls an insertion and removal of the linkage device with respect to the hollow shaft.

The at least one probe connector slot can include a ramp for applying a force in an axial direction for abutting the probe connector with an end of the hollow shaft.

The hollow shaft of the rotary motor can include a threaded region, and the probe connector can comprise a thread that mates with the threaded region of the rotary motor.

The system can further comprise a sensor at the first coupling mechanism that detects when the handle is coupled at the first coupling mechanism, and wherein the translation table moves the rotary motor relative to the probe connector for coupling the threaded probe connector with the threaded region of the rotary motor.

The system can further comprise a linear motor that translates the at least one fiber in a linear direction along the longitudinal axis.

The motion unit can further comprise a translation table that is moved along the base by the linear motor in the linear direction along the longitudinal axis.

The system can further comprise a locking mechanism coupled to the translation table, and an actuator coupled to the base, and the locking mechanism can engage the actuator to prevent the translation table from a linear movement.

The system can be constructed and arranged to produce surface temperature estimations of a body cavity having a tissue surface.

The system can further comprise a sensor assembly having a sensor that receives the infrared energy from the at least one fiber, and convert the received infrared energy into temperature information signals.

The sensor assembly can be positioned on a positioning plate for aligning the sensor assembly with a proximal end of the at least one fiber.

The positioning plate can include a positioning plate for adjusting the sensor assembly in at least one of an pitch, yaw, roll, x, y, and z direction relative to the proximal end of the at least one fiber.

The sensor assembly can comprise a cooling assembly constructed and arranged to cool one or more portions of the sensor.

The system can further comprise a controller that processes the infrared energy received by the sensor assembly and generates an output that includes temperature data related to the processed infrared energy.

A portion of the fiber assembly between the probe connector and the first coupling assembly can extend in the linear direction along the longitudinal axis during translation of the at least one fiber.

The at least one fiber can extend directly between the first coupling assembly and the motion unit.

The fiber assembly can be passive, and constructed and arranged to only collect infrared energy from the tissue surface.

The first coupling mechanism can include a sheath bulkhead coupled to the base and having a slot for receiving the handle of the probe assembly.

The sheath bulkhead can include a twist lock coupling at the slot, and wherein the handle includes a bayonet portion that mates with the twist lock coupling at the slot to prevent rotation of the handle about the longitudinal axis.

The twist lock coupling can include a spring-loaded pin activation element and the bayonet portion of the handle includes at least one lobe, and wherein the spring-loaded pin activation element biases the at least one lobe at the sheath bulkhead unit.

The motion unit can comprise a Yankee screw and a rotary motor, wherein the Yankee screw includes a Yankee screw motor that translates the at least one fiber and the rotary motor in a linear direction along the longitudinal axis.

The Yankee screw motor can operate to rotate the Yankee screw.

The Yankee screw can include dual opposed continuous helical grooves and the Yankee screw motor can rotates the Yankee screw to translate the at least one fiber and the rotary motor in the linear direction.

A translation speed and a rotational speed of the fiber assembly can be both driven by the rotary motor.

The at least one fiber can collect infrared energy from a body lumen tissue surface while the rotary motor of the motion unit rotates the at least one fiber about the longitudinal axis.

The at least one fiber can collect infrared energy from a body lumen tissue surface while the motion unit at least one of translates the at least one fiber along the longitudinal axis and rotates the at least one fiber about the longitudinal axis.

The system can further comprise a controller that processes Infrared energy collected by the at least one fiber, and generates an output that includes temperature data related to the processed Infrared energy.

The output can include at least one of a two dimensional (2D) graphical temperature map, a one dimensional (1D) graphical temperature map, a temperature value, an alarm, and a temperature rate of change.

The probe assembly can further comprise a sheath coupled to the handle, wherein a distal end of the fiber is positioned in the sheath and at least one of translates and rotates relative to the sheath.

The system can further comprise at least one marker band positioned at a distal end of the sheath. The distal end of the fiber assembly can be constructed and arranged to translate relative to the at least one marker band.

The sheath can include an IR opaque region at a distal side of the marker band, and an IR transmissive region at a proximal side of the marker band.

The at least one marker band can comprise a distal band and a proximal band, and wherein the first fiber assembly is constructed and arranged to translate between the distal band and the proximal band.

The translation assembly can be constructed and arranged to translate the fiber in a reciprocating motion between the distal band and the proximal band, and wherein the fiber receives the infrared energy from a region between the distal band and the proximal band.

The at least one marker band can be constructed and arranged to cause a sensor in communication with a proximal end of the at least one fiber to produce a predetermined signal when the distal end of the at least one fiber receives infrared light from the at least one marker band.

The at least one marker band can be C-shaped, and the C-shaped marker band can include two ends, and a gap between the two ends.

The gap can identify a rotational position of the at least one fiber.

The gap can provide a different and distinguishable signal from the rest of the marker band due to differences in emissivity between tissue and the marker band material.

The system can further comprise a processor that converts the infrared energy received at the at least one fiber into a plurality of temperature measurements.

The system can further comprise a display user interface that receives the temperature measurements from the processor, and displays a graphical temperature map corresponding to the tissue surface.

The user interface can be constructed and arranged to display the temperature map of at least one of a one-dimensional, two-dimensional, and three-dimensional representation of the tissue surface.

The user interface can be constructed and arranged to display the temperature map of a four-dimensional representation of the tissue surface.

The user interface can be constructed and arranged to display other temperature information.

The other temperature information can comprise at least one of peak temperature information, rate of change of temperature information, and average temperature information for multiple tissue surfaces.

According to another aspect, a probe assembly, comprises a rotary motor having a rotatable hollow shaft extending along a longitudinal axis; an optical device extending through the hollow shaft along the longitudinal axis; a stationary fiber assembly in communication with the optical device; a mounting sleeve coupled to the hollow shaft along the longitudinal axis; and an optical element in a mounting sleeve, the optical element in direct communication with a distal end of the optical device for outputting received infrared energy to the distal end of the optical device, wherein the rotary motor rotates the hollow shaft relative to the fiber assembly along the longitudinal axis, and wherein the hollow shaft rotates the mounting sleeve about the longitudinal axis relative to the stationary fiber assembly.

The probe assembly can further comprise a probe sheath about the rotary motor and mounting sleeve, the probe sheath include an infrared transmissive surface, wherein the optical element can receive the infrared energy from a tissue surface via the infrared transmissive surface.

The optical device can be a portion of the fiber assembly, and wherein the rotary motor rotates the hollow shaft about the fiber assembly.

The probe assembly can further comprise a slip ring about at least a portion of the stationary fiber assembly, the slip ring positioned between the stationary fiber assembly and the hollow shaft.

The slip ring can be coupled to an exposed region of the hollow shaft at a proximal end of the rotary motor to align a combination of the optical element, the fiber assembly, and a stationary optical element adjacent a proximal end of the fiber assembly.

The probe assembly can further comprise a separating element between the rotary motor and the mounting sleeve that surrounds an exposed region of the hollow shaft extending from the rotary motor.

The separating element can include a lubricous material, bearing, or a running gap.

The optical device can include an index-matched optical element between the fiber assembly and the optical element, and the optical element can direct Infrared energy along the index-matched optical element to the fiber assembly.

The probe assembly can further comprise an electrical connector for providing power to the rotary motor.

In another aspect, a temperature mapping system that produces temperature estimations of a tissue surface, comprises a probe assembly; a fiber assembly extending through the probe assembly, the fiber assembly including at least one fiber constructed and arranged to receive infrared energy from the tissue surface; a processor that converts the received infrared energy into temperature information signals; and a motion unit coupled to the proximal end of the probe assembly, the motion unit constructed and arranged to at least one of rotate the at least one fiber about a longitudinal axis and translate the fiber assembly along the longitudinal axis at a speed that changes according to the temperature signals.

The processor can process an amount of temperature data that is dependent on a rate of rotation and speed of translation of the fiber assembly by the motion unit.

The motion unit can increase a rotational speed of the fiber assembly when an area of interest at the tissue surface is identified.

The motion unit can decrease the translation speed of the fiber assembly and reduces a translation distance to the area of interest.

The motion unit can further increase the rotational speed of the fiber assembly.

The motion unit can proportionally increase the translation speed of the fiber assembly and increase the rate of rotation of the fiber assembly at or near the area of interest.

In another aspect, a system that produces temperature estimations of a tissue surface comprises a monitoring unit that receives and displays the temperature information; a probe assembly; a fiber assembly extending through the probe assembly, the fiber assembly including at least one fiber constructed and arranged to receive infrared energy from the tissue surface; a patient interface unit, comprising a base; a motion unit at the base, the motion unit constructed and arranged to at least one of rotate the at least one fiber relative to the base about the longitudinal axis and translate the at least one fiber relative to the base in a linear direction along the longitudinal axis; a first coupling mechanism coupled to the base; and a second coupling mechanism at the motion unit, wherein the probe assembly is removably coupled to each of the first and second coupling mechanisms; and a processor that converts the infrared energy received at the at least one fiber into a plurality of temperature measurements.

The patient interface unit can comprise a sensor assembly co-located with the rotary motor on the translation table.

In another aspect, a method of controlling a temperature measurement probe comprises: determining a first longitudinal position and a second longitudinal position of a distal end of a probe sheath, the first and second longitudinal positions spaced apart from each other in the longitudinal direction, a first region of interest being defined therebetween; collecting, at a fiber extending through the probe sheath, data from tissue proximal the probe sheath in the first region of interest; determining a second region of interest within the first region of interest, in response to the collected data; and controlling a rate of movement of the fiber at a collection region to be different when collecting data within the second region of interest as compared to collecting data that lies within the first region of interest and beyond the second region of interest.

According to another aspect, a system for performing a medical procedure comprises a base; a probe assembly having a proximal end and a distal end, the proximal end of the probe assembly at the base and extending along a longitudinal axis, and including: a handle at the proximal end of the probe assembly; and a probe connector; a fiber assembly extending through the probe assembly, the fiber assembly including at least one fiber constructed and arranged to receive infrared energy from the tissue surface; a motion unit at the base, the motion unit constructed and arranged to at least one of rotate the at least one fiber relative to the base about the longitudinal axis and translate the at least one fiber relative to the base in a linear direction along the longitudinal axis; a first coupling mechanism coupled to the base, wherein the handle is removably coupled to the first coupling mechanism; and a second coupling mechanism at the motion unit, wherein the probe connector is removably coupled to the second coupling mechanism.

In another aspect, provided is a method for performing a medical procedure using the surgical instrument referred to herein.

### BRIEF DESCRIPTION OF THE DRAWINGS

The accompanying drawings, which are incorporated in and constitute a part of this specification, illustrate various embodiments of the present inventive concepts, and together with the description, serve to explain the principles of the inventive concepts. In the drawings:

FIG. 1 is a schematic view of a temperature mapping system including a temperature measurement probe, consistent with the present inventive concepts.

FIG. 2 is a sectional side view of the distal portion of the temperature measurement probe of FIG. 1 positioned in a body lumen, consistent with the present inventive concepts.

FIG. 2A is a magnified sectional side view of the distal portion of the temperature measurement probe of FIG. 2, including an infrared light collector, consistent with the present inventive concepts.

FIG. 2B is a perspective view of a component of the infrared light collector of FIG. 2A, including the pathway of collected infrared light, consistent with the present inventive concepts.

FIG. 3 is an optical schematic of a “close-optimized” optical system, including cross sectional representations of tissue surface areas, consistent with the present inventive concepts.

FIG. 4 is an optical schematic of a “range-optimized” optical system, including cross sectional representations of tissue surface areas, consistent with the present inventive concepts.

FIG. 5A is a perspective view of a sensor assembly and a rotating assembly, consistent with the present inventive concepts.

FIG. 5B is a perspective cross sectional view of the rotating assembly of FIG. 5A, consistent with the present inventive concepts.

FIG. 6 is a perspective view of a translating assembly, consistent with the present inventive concepts.

FIG. 7 is an optical schematic of an optical pathway proximate a sensor assembly, consistent with the present inventive concepts.

FIG. 8A is an optical schematic of an infrared detector illustrating projections of infrared light focused toward the detector, in a configuration that overfills the detector, consistent with the present inventive concepts.

FIG. 8B is an optical schematic of an infrared detector illustrating projections of the infrared light focused toward the detector, in a configuration that underfills the detector, consistent with the present inventive concepts.

FIG. 9 is a schematic view of a temperature measurement system, consistent with some embodiments of the present inventive concepts.

FIG. 10 is a perspective view of patient interface unit of FIG. 9, consistent with some embodiments of the present inventive concepts.

FIG. 11 is a perspective partial cross-sectional view of elements of patient interface unit of FIGS. 9 and 10, consistent with some embodiments of the present inventive concepts.

FIG. 12 is a close-up perspective cross-sectional view of a region between the detector and the rotary motor assembly of patient interface unit of FIGS. 9-11, consistent with some embodiments of the present inventive concepts.

FIG. 13 is a close-up perspective cross-sectional view of a latch mechanism of patient interface unit of FIGS. 9-12, consistent with some embodiments of the present inventive concepts.

FIG. 14A is a partial cutaway perspective view of a patient interface unit and a probe connector separate from each other, and further illustrating a view of an interior of a sheath attachment frame of patient interface unit, consistent with some embodiments of the present inventive concepts.

FIG. 14B is a perspective partial cross-sectional view of elements of patient interface unit of FIG. 14A, consistent with some embodiments of the present inventive concepts.

FIG. 14C is a perspective view of the probe assembly of FIGS. 14A and 14B.

FIG. 14D is a perspective view of the probe assembly of FIGS. 14A-14C, wherein the handle is separate from the probe coupling.

FIG. 15 is a close-up perspective view of a region of the rotary motor assembly receiving probe connector of FIGS. 14A and 14B, exposing an interior of a spring-loaded actuator coupling, consistent with some embodiments of the present inventive concepts.

FIG. 16A is a cross-sectional view of a patient interface unit, illustrating a loading of a probe connector, consistent with some embodiments of the present inventive concepts.

FIG. 16B is a cross-sectional view of patient interface unit of FIG. 16A, illustrating an engaging of a probe connector, consistent with some embodiments of the present inventive concepts.

FIG. 16C is a perspective view of patient interface unit of FIGS. 16A and 16B, consistent with some embodiments of the present inventive concepts.

FIG. 17A is a perspective view of a patient interface unit, consistent with some embodiments of the present inventive concepts.

FIG. 17B is a close-up perspective view of a rotary motor assembly of patient interface unit of FIG. 17A, consistent with some embodiments of the present inventive concepts.

FIG. 17C is a cross-sectional view of patient interface unit of FIGS. 17A and 17B, consistent with some embodiments of the present inventive concepts.

FIG. 17D is a close-up cross-sectional view of patient interface unit of FIGS. 17A-17C, consistent with some embodiments of the present inventive concepts. FIG. 18A is a perspective view of a patient interface coupling between a rotary motor assembly and a probe connector, consistent with other embodiments of the present inventive concepts.

FIG. 18B is a cross-sectional view of patient interface coupling of FIG. 18A, consistent with some embodiments of the present inventive concepts.

FIG. 19 is a close-up view of a threaded coupling between a probe and a patient interface unit, consistent with some embodiments of the present inventive concepts.

FIG. 20A is a perspective view of a distal end of probe, consistent with some embodiments of the present inventive concepts.

FIG. 20B is a cross-sectional view of the distal end of probe of FIG. 20A, consistent with some embodiments of the present inventive concepts.

FIG. 21 is a cross-sectional view of the distal end of a probe, consistent with some embodiments of the present inventive concepts.

FIG. 22 is a view of a patient interface unit, consistent with some embodiments of the present inventive concepts.

FIG. 23 is a cross-sectional view of a probe having a fiber assembly in a first position relative to a distal marker band, consistent with some embodiments of the present inventive concepts.

FIG. 24 is a cross-sectional view of probe of FIG. 23, wherein the fiber assembly is in a second position relative to the distal marker band, consistent with some embodiments of the present inventive concepts.

FIG. 25 is a perspective view of a probe configured to include a C-shaped marker band about a sheath, consistent with some embodiments of the present inventive concepts.

FIGS. 26A and 26B are graphs illustrating locations of a fiber assembly relative to a C-shaped marker band of FIG. 26, consistent with some embodiments of the present inventive concepts.

FIG. 27 is an illustration of a display at a monitoring unit, consistent with some embodiments of the present inventive concepts.

FIG. 28 is an illustration of two dimensional (2D) and one dimensional (1D) temperature maps, respectively, produced in response to an IR scan of a tissue surface, consistent with some embodiments of the present inventive concepts.

FIG. 29 is a view of a probe engaged in a multi-mode scanning operation, consistent with some embodiments of the present inventive concepts.

FIG. 30 is a schematic view of a temperature measurement system, consistent with some embodiments of the present inventive concepts.

FIG. 31 is a cross-sectional view of the calibration unit of FIG. 30.

### DETAILED DESCRIPTION

Reference will now be made in detail to the present embodiments of the inventive concepts, examples of which are illustrated in the accompanying drawings. Wherever possible, the same reference numbers will be used throughout the drawings to refer to the same or like parts.

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

For example, it will be appreciated that all features set out in any of the claims (whether independent or dependent) can be combined in any given way.

Provided herein is a temperature measurement system for producing a temperature map for multiple locations, such as a two or three dimensional surface of a patient's tissue. The system can include one or more sensors, such as infrared (IR) light detectors or other infrared sensors. In other embodiments, the system can include thermistor or thermocouple sensors. The system can include a reusable portion, and one or more disposable portions. The system can include a probe, such as a probe constructed and arranged to be inserted into a body lumen such as the esophagus, respiratory tract, or colon. Probe can include an elongate member such as a shaft, and the system can be constructed and arranged to measure temperature at multiple tissue locations positioned at the side of the elongate member and/or forward of the distal end of the elongate member. The system or probe can be constructed and arranged as described in applicant's co-pending International Patent Application Serial Number PCT/US2011/061802, entitled “Ablation and Temperature Measurement Devices”, and filed Nov. 22, 2011, the contents of which is incorporated by reference in its entirety.

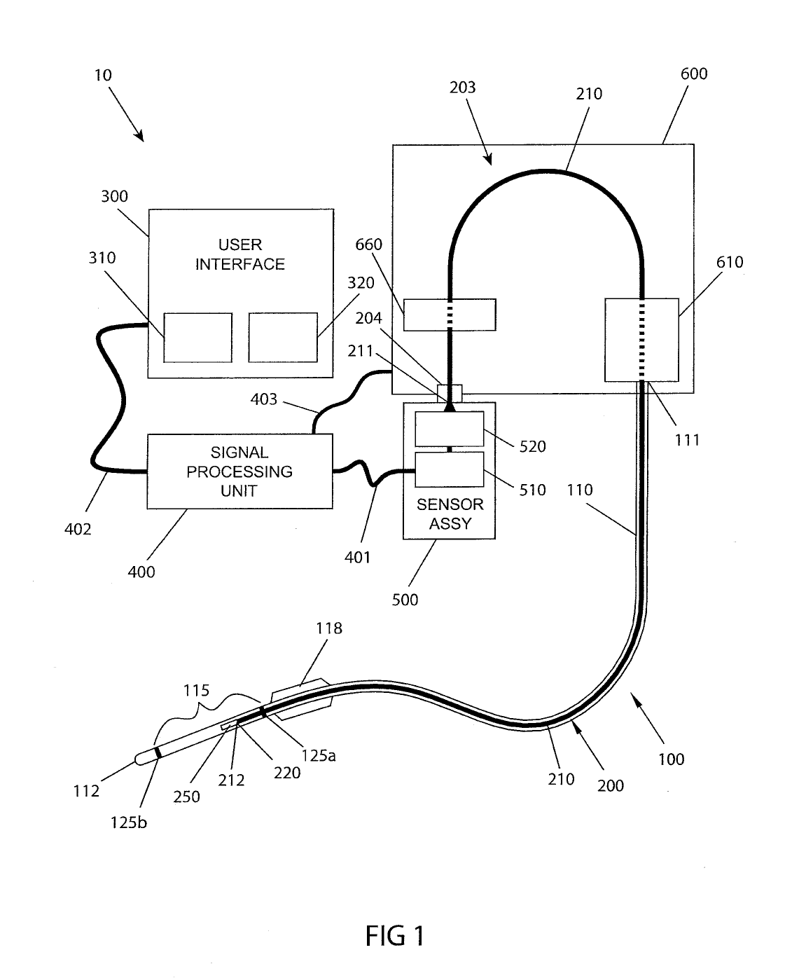

Referring now to FIG. 1, a schematic view of a temperature mapping system including a temperature measurement probe is illustrated, consistent with the present inventive concepts. System 10 includes probe 100, sensor assembly 500, signal processing unit (SPU) 400, and user interface 300. Probe 100 includes shaft 110 which slidingly receives an elongate filament, fiber assembly 200. Fiber assembly 200 is constructed and arranged to collect at least infrared light emanating from one or more surface locations (e.g. one or more tissue surface locations) positioned radially out from the central axis of the distal portion of shaft 110. The collected infrared light travels proximally within fiber assembly 200 and is received by sensor assembly 500. Sensor assembly 500 converts the received infrared light to one or more information signals that are transmitted to SPU 400. System 10 can include motion transfer assembly 600, configured to cause fiber assembly 200 to translate and/or rotate, such as to collect infrared light from a series of tissue locations (e.g. a contiguous or discontiguous surface of tissue). SPU 400 converts the one or more information signals received from sensor assembly 500 into a series of temperature measurements that can be correlated to the series of tissue locations, such as to provide information regarding temperatures (e.g. average temperatures) present on a two and/or three dimensional tissue surface.

Shaft 110 includes proximal end 111 and distal end 112. Distal end 112 can comprise a rounded tip configured as shown for atraumatic insertion of probe 100 into a body lumen of a patient. Shaft 110 can comprise a material selected from the group consisting of: polyethylene; polyimide; polyurethane; polyether block amide; and combinations of these. Shaft 110 can comprise a braided shaft and/or include one or more braided portions constructed and arranged to provide increased column strength and/or improve response to a torque applied at or near proximal end 111 of shaft 110. Probe 100 can be configured for insertion over a guidewire, not shown but typically where shaft 110 includes a guidewire lumen or distal guidewire sidecar as is known to those of skill in the art. The distal portion of shaft 110 includes a relatively infrared transparent tube (i.e. an infrared transmissive tube), window 115, comprising a tubular segment which can include at least a portion which is transparent or relatively transparent to infrared light. Window 115 can comprise a material selected from the group consisting of: polyethylene such as high density polyethylene (HDPE) or low density polyethylene (LDPE); germanium or similarly infrared transparent materials; and combinations of these. In embodiments where shaft 110 includes a braid or other reinforcing structure, window 115 or a portion of window 115 can be void of the reinforcing structure.

Shaft 110 can be rigid, flexible, or include both rigid and flexible segments along its length. Fiber assembly 200 can be rigid, flexible, or include both rigid and flexible segments along its length. Shaft 110 and fiber assembly 200 can be constructed to be positioned in a straight or curvilinear geometry, such as a curvilinear geometry including one or more bends with radii less than or equal to 4 inches, less than or equal to 2 inches, or less than or equal to 1 inch, such as to allow insertion into the esophagus via a nasal passageway. In some embodiments, shaft 110 and fiber assembly 200 comprise sufficient flexibility along one or more portions of their length to allow insertion of probe 100 into a body lumen or other body location, such as into the esophagus via the mouth or a nostril, the respiratory tract via the mouth or a nostril, or into the lower gastrointestinal tract via the anus, and/or into the urethra. Shaft 110 can comprise an outer diameter less than 15Fr, such as a shaft with a diameter less than 12Fr, less than 9Fr, or less than 6Fr.

Fiber assembly 200 includes fiber 210 comprising proximal end 211 and distal end 212. Connector 204 is positioned on proximal end 211 and configured to mechanically and optically connect fiber assembly 200 to sensor assembly 500. In some embodiments, connector 204 comprises a linearly adjustable table or a two-dimensionally adjustable (X-Y) table constructed and arranged to allow precise positioning of fiber 210 relative to one or more components of sensor assembly 500. In some embodiments, one or two dimensional positioning can be performed by the manufacturer only. Fiber 210 can comprise one or more materials highly transparent to one or more ranges of infrared light wavelengths, such as one or more fibers comprising a material selected from the group consisting of: zinc selenide; germanium; germanium oxide; silver halide; chalcogenide; a hollow core fiber material; and combinations of these. Fiber 210 can be configured to be highly transparent to infrared light with wavelengths between 6 μm to 15 μm, or between 8 μm and 11 μm. In some embodiments, fiber 210 comprises multiple fibers, such as multiple fibers in a coherent or non-coherent bundle.

In some embodiments, proximal end 211 and/or distal end 212 of fiber 210 comprises a surface with a coating, such as an anti-reflective (AR) coating. System 10 can include one or more components that include an optical surface that receives infrared light and/or from which infrared light is emitted. These optical surfaces can include one or more anti-reflective coatings, such as a coating selected from the group consisting of: a broadband anti-reflective coating such as a coating covering a range of 6 μm-15 μm or a range of 8 μm-11 μm; a narrow band anti-reflective coating such as a coating covering a range of 7.5 μm-8 μm or a range of 8 μm-9 μm; a single line anti-reflective coating such as a coating designed to optimally reflect a single wavelength or a very narrow range of wavelengths in the infrared region; and combinations of these. Anti-reflective coatings can be included to improve transmission by up to 30% per surface by reducing Fresnel losses at each surface. Anti-reflective coatings can be constructed and arranged to accept a small or large range of input angles.

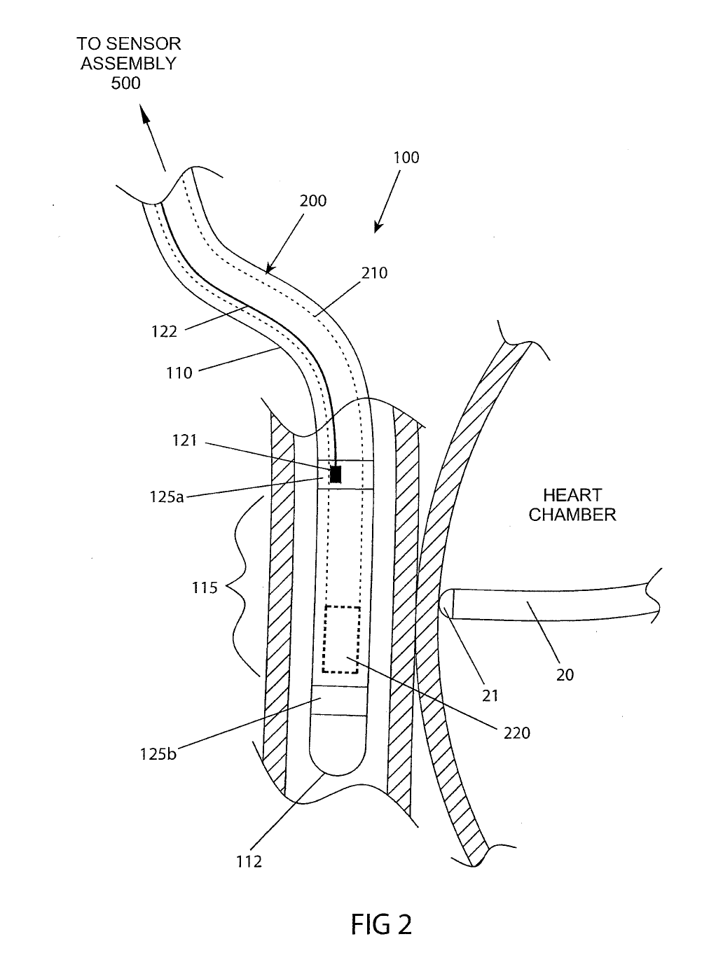

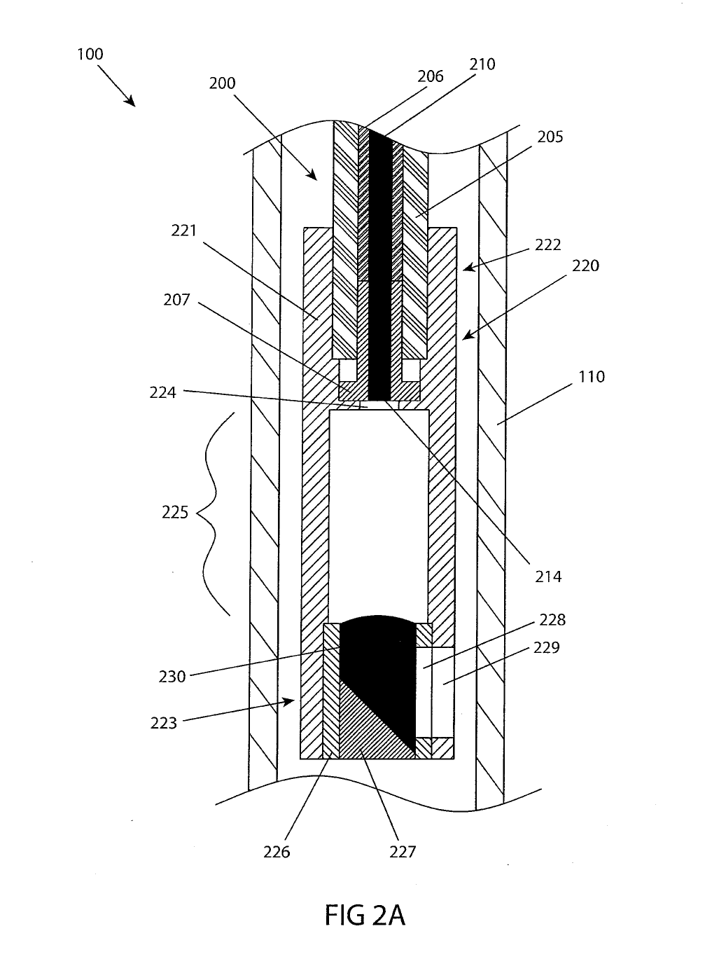

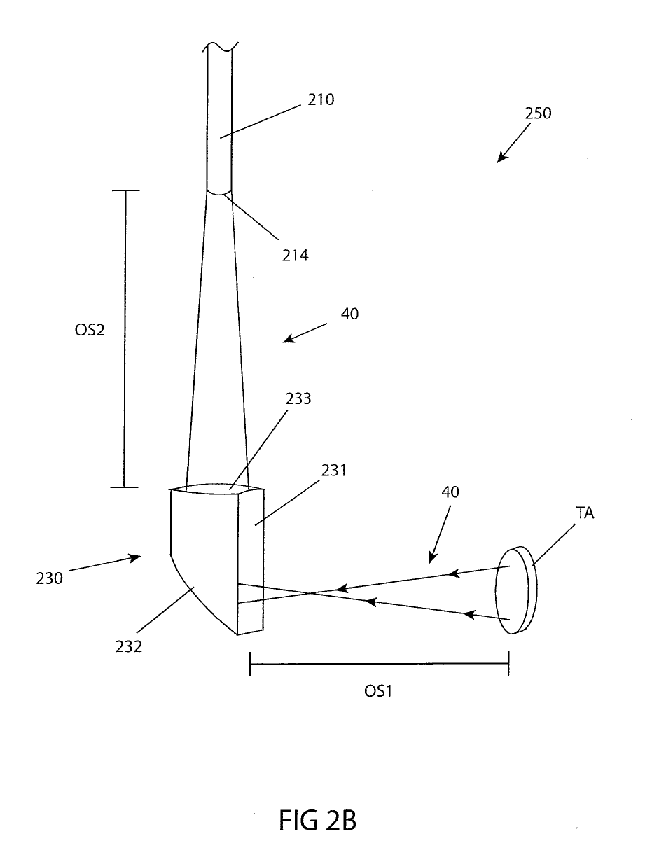

In some embodiments, fiber assembly 200 comprises a cladding, such as is described in reference to FIG. 2A herebelow. Cladding can be included to cause and/or maintain total internal reflection of the infrared light as it travels from the distal to proximal end of fiber assembly 200. Alternatively or additionally, fiber assembly 200 can comprise a coil, braid or other twist resisting structure surrounding optical fiber 210, such as to improve torsional response of fiber assembly 200. In some embodiments, fiber assembly 200 comprises a coil, braid or other surrounding element (e.g. a torque shaft) for improving torque response, such as is described in reference to FIG. 2A herebelow.

System 10 includes an optical assembly 250 comprising collector 220, which can be attached to distal end 212 of fiber 210. Collector 220 can include one or more optical components, such as one or more optical components used to perform an action on the collected infrared light, such as an action selected from the group consisting of: focus; split; filter; transmit without filtering (e.g. pass through); amplify; refract; reflect; polarize; and combinations of these. Collector 220 can include one or more optical components selected from the group consisting of: optical fiber; lens; mirror; filter; prism; amplifier; refractor; splitter; polarizer; aperture; optical frequency multiplier and combinations of these. Collector 220 can include a housing and other mechanical, electrical and/or optical components, such as are described in reference to collector 220 of FIG. 2A herebelow. Collector 220 can comprise a finite rigid length, such as a rigid length less than 3 cm, less than 2 cm, less than 1 cm or less than 0.5 cm, such as to accommodate travel through a curvilinear path as described hereabove.

Infrared light which is emitted from a particular tissue location proximate to the distal portion of fiber assembly 200, and then passes through window 115, is collected by collector 220. Collector 220 is optically coupled to fiber 210 at distal end 212, such that the collected light travels proximally through fiber 210. Proximal end 211 is optically coupled to sensor assembly 500 such that the collected light is received by sensor assembly 500. A signal produced by sensor assembly 500 based on the collected light is correlated by SPU 400 to an estimated, average temperature, hereinafter “measured temperature”, for that particular tissue location, hereinafter the “collection location”. This measured temperature represents an average temperature of the entire surface of the collection location, which can include multiple different temperatures across its entire surface. In other words, the collected infrared light from each collection location travels proximally through fiber 210 as a single, undividable signal correlating to an average temperature of the entire collection location. Errors in the measured temperature can be caused by a factor selected from the group consisting of: unaccounted for and/or unknown infrared signal losses along system 10's optical pathway; unaccounted for and/or unknown infrared signal gains (e.g. an extraneous input of infrared light) along system 10's optical pathway; sensor assembly 500 inaccuracies or spurious signals; electrical signal noise; and combinations of these.

In some embodiments, collector 220 is constructed and arranged to collect light from a collection location (e.g. a tissue surface area) with an area of approximately 0.5 mm²-1.5 mm², such as an area of approximately 1.0 mm². In some embodiments, collector 220 is constructed and arranged to collect light from a relatively circular shaped area with an equivalent diameter ranging between 0.5 mm and 1.5 mm. In some embodiments, collector 220 is constructed and arranged to collect light from a rectangular or elliptical shaped area with a major axis between 0.5 and 1.5 mm. Collection locations can comprise a broad range of sizes and shapes, such as locations comprising an area between 0.1 mm² and 20 mm². Collection locations can comprise various shapes such as a shape selected from the group consisting of: an ellipse such as a circle or an oval; a rectangle such as a square; a polygon such as a trapezoid; and combinations of these. The efficiency of collection of light from the collection location can vary over the collection area, for example the efficiency of collection from the center of the collection location can be greater than that from the periphery of the collection location, resulting in a weighting of the measured temperature towards that of the center of the collection location. System 10 can be constructed and arranged to collect light from multiple tissue surface areas, such as by rotating and/or spinning collector 220 as described in detail herein.

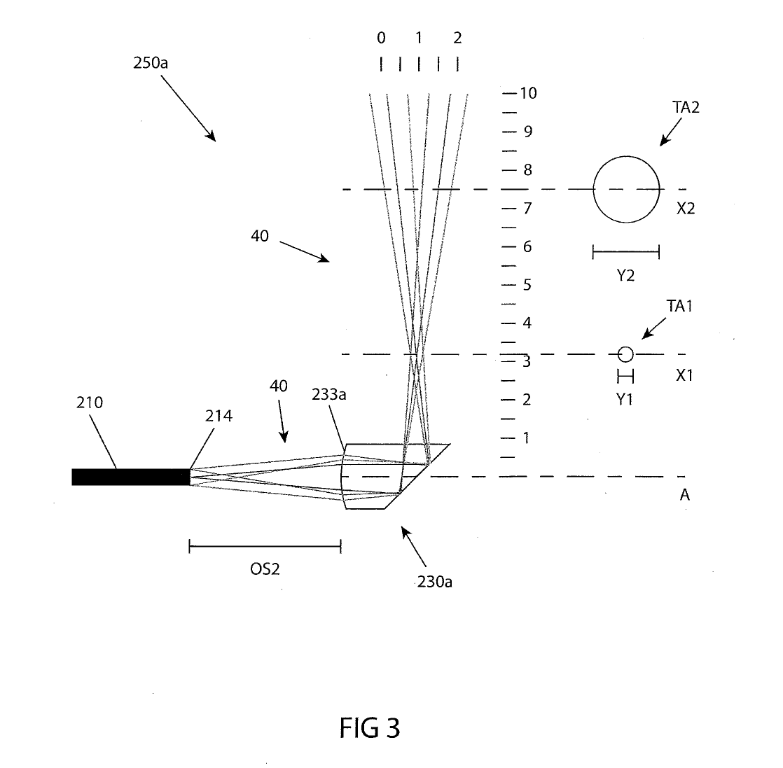

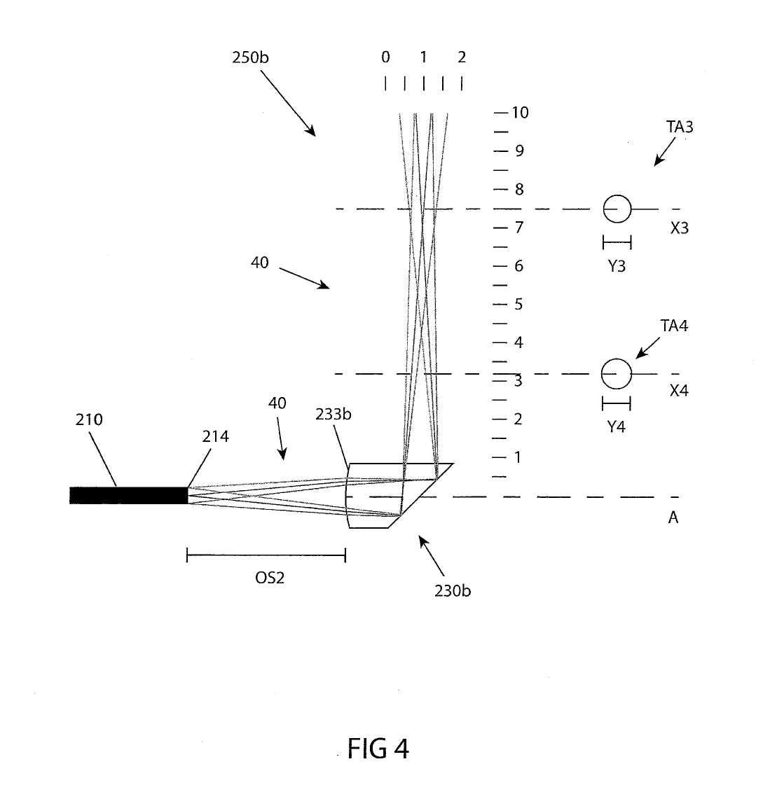

A collection location and/or groups of collection locations can comprise tissue that is relatively flat (e.g. the included tissue surface orthogonal to collector 220 has a relatively constant distance to collector 220), or it can comprise tissue that is undulating or otherwise includes peaks and/or valleys. System 10 can be configured to minimize temperature measurement errors by optics whose focus matches the topography of the tissue surface being measured. A non-limiting example of a system 10 optimized for tissue at a relatively uniform distance from probe 100 is described in reference to FIG. 3 herebelow. A non-limiting example of a system 10 optimized for tissue at a varying or unknown distance from probe 100 is described in reference to FIG. 4 herebelow.

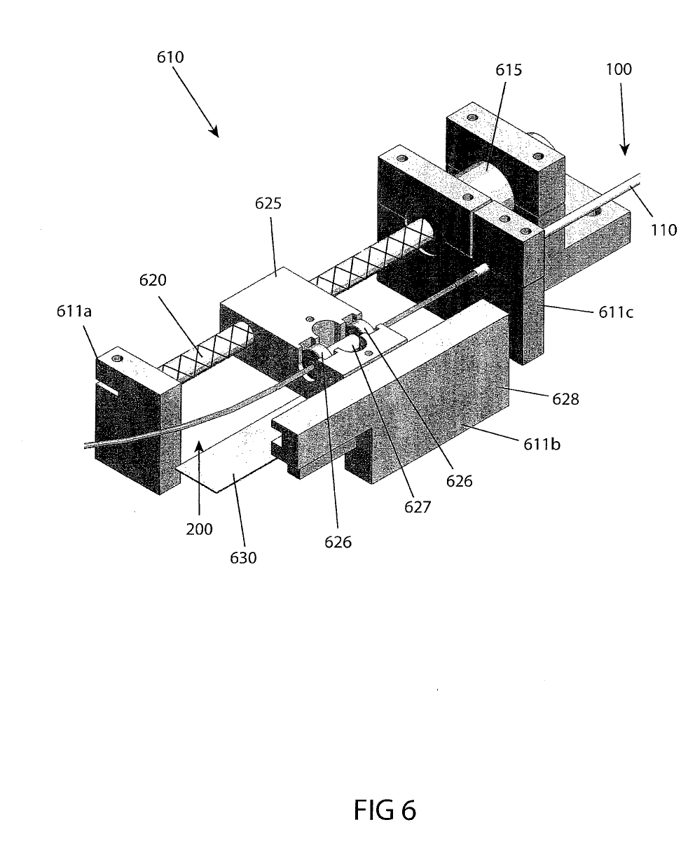

As described above, in some embodiments, fiber assembly 200, including collector 220, is configured to be translated and/or rotated, such as by translating assembly 610 and/or rotating assembly 660, respectively. Translating assembly 610 operably engages an axial segment of fiber assembly 200, and applies an axial force to cause fiber assembly 200 to move forward and back within shaft 110. Translating assembly 610 can be configured to create a reciprocating motion between 5 mm and 100 mm, such as between 10 mm and 40 mm, such as a reciprocating translation of approximately 25 mm in each direction. In some embodiments, the magnitude of reciprocating motion is constructed and arranged to collect temperature information from a sufficient length of the esophagus during a cardiac ablation procedure. Fiber assembly 200 can comprise service loop 203, which comprises at least a flexible portion and is positioned and arranged to accommodate the translating motion without detaching from or otherwise imparting an undesired force to rotating assembly 660 and/or sensor assembly 500 (e.g. to accommodate the translating motion of fiber assembly 200). In some embodiments, translating assembly 610 includes one or more linear encoders or other position sensors constructed and arranged to produce a signal correlating to a linear position of fiber assembly 200. In some embodiments, translating assembly 610 is constructed and arranged as described in reference to FIG. 6 herebelow.

Rotating assembly 660 operably engages another axial segment of fiber assembly 200, and applies a rotational force to cause fiber assembly 200 and collector 220 to rotate, such as a continuous 360° rotation or a partial circumferential rotation (e.g. 45° to 320° reciprocating rotation). In an alternative embodiment, rotating assembly 660 is positioned distal to collector 220, distal position not shown but typically comprising a rotary motor positioned proximate distal end 212 and operably coupled to collector 220 such that at least a portion of collector 220 can be rotated without rotating fiber 210.

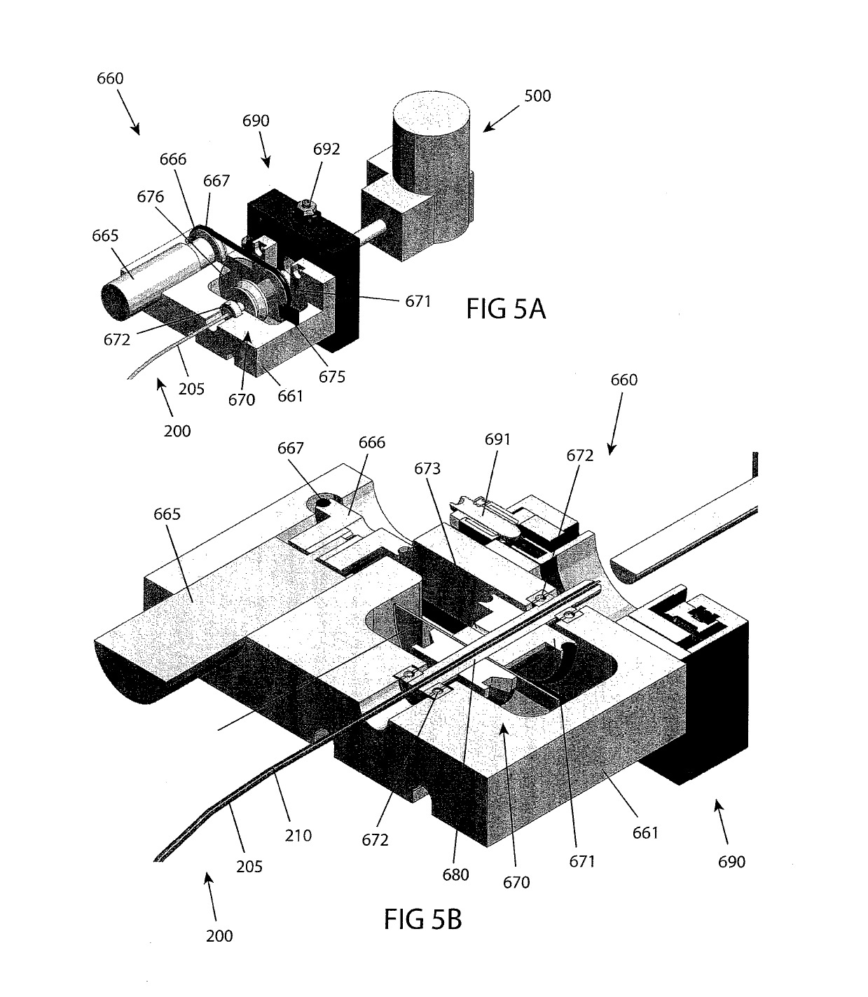

In some embodiments, rotating assembly 660 includes one or more rotary encoders or other position sensors constructed and arranged to produce a signal correlating to a rotational position of collector 220 and/or fiber assembly 200. In some embodiments, rotating assembly 660 is constructed and arranged as described in reference to FIG. 5A herebelow.

In some embodiments, rotating assembly 660 and/or sensor assembly 500, are positioned on or otherwise coupled to translating assembly 610, such that rotating assembly 660 and/or sensor assembly 500 translate along with fiber assembly 200. In these embodiments, service loop 203 can be avoided, such as to reduce the length of fiber assembly 200 and/or to reduce or eliminate any signal losses that occur during the flexing of service loop 203.

In some embodiments, translation and rotation occur simultaneously, such that the infrared light collected by collector 220 represents light collected from a helical pattern of collection locations. In other embodiments, a rotation (e.g. a 360° rotation of collector 220), is sequentially followed by a translation (e.g. an advancement or a retraction of collector 220), and the rotation-translation is repeated such that the infrared light collected represents a series of collection locations with a geometry comprising multiple two dimensional, parallel circles.

The information provided by sensor assembly 500 is used by SPU 400 to produce a table of collection location measured temperatures, which represent an estimated, averaged temperature for the collection location, as described above. The table provided by SPU 400 can be represented (e.g. by user interface 300) in the form of a temperature map correlating to the geometry of the multiple collection locations. In some embodiments, the multiple collection locations comprise a segment of tubular tissue, such as a segment of esophagus, and the temperature map is a two dimensional representation of the “unfolded” luminal wall or other body tissue. In other embodiments, a three dimensional representation of the luminal wall or other body tissue can be provided. The table or other representation can be updated on a regular basis, such as via data collected during a series of reciprocating translations in which collector 220 is continuously or semi-continuously rotated.

In some embodiments, a single forward or reverse translation over approximately 25 mm occurs over a time period of between 0.1 seconds and 30 seconds, such as a time period between 0.2 seconds and 5.0 seconds, such as a time period between 0.5 seconds and 2.0 seconds, such as a time period of approximately 1.0 second. During the forward or reverse translation, collector 220 can be rotated, such as at a rotational velocity between 1000 rpm and 15000 rpm, or between 4000 rpm and 8000 rpm, such as approximately 7260 rpm. In some embodiments, a forward or reverse translation is separated by a reverse or forward translation, respectively, after a period of time. In other embodiments, a forward or reverse translation is initiated relatively immediately after the completion of the previous reverse or forward translation, respectively.

Sensor assembly 500 comprises one or more sensors configured to produce a signal based on the infrared light received from fiber assembly 200. As described above, the received infrared light can represent transmission of infrared light collected from a series of collection locations, as determined by translation and/or rotation of collector 220. SPU 400 can be configured to correlate the signals produced by sensor assembly 500 into a table of temperature values associated with a series of collection locations. Sensor assembly 500 can comprise a finite response time (e.g. a delay of output signal availability of one or more electronic components), during which a signal produced by sensor assembly 500, based on received infrared light, is unavailable (e.g. not accurate). In these embodiments, SPU 400 can be configured to discretely sample sensor assembly 500 to accommodate for any signal availability delay.

Sensor assembly 500 can include IR detector 510 such as an element selected from the group consisting of: a photoconductor such as a mercury cadmium telluride photodetector or a mercury zinc telluride photodetector; a microbolometer; a pyroelectric detector such as a lithium tantalite detector or triclycine sulfate detector; a thermopile; and combinations of these. In some embodiments, detector 510 comprises a response time less than 200 milliseconds, such as less than 1 millisecond.

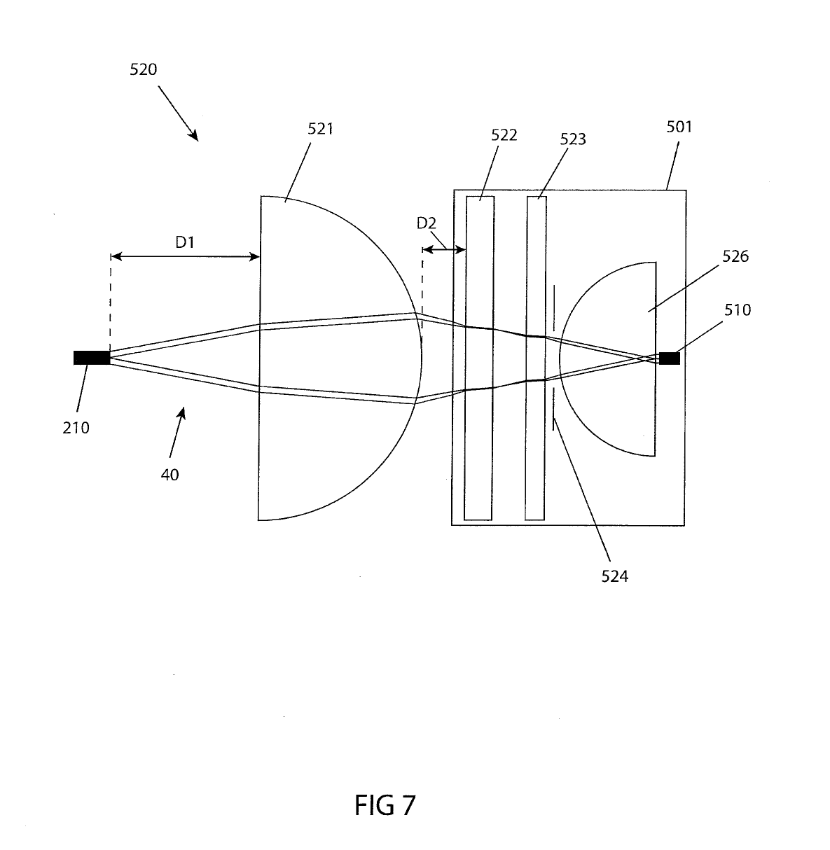

Sensor assembly 500 or another assembly of system 10 can include an optical assembly 520 comprising one or more optical components constructed and arranged to focus infrared light received from fiber assembly 200 onto IR detector 510. In some embodiments, optical assembly 520 is configured as described in reference to FIG. 7 herebelow.

IR detector 510 can be configured to convert the received infrared light into an electrical signal, such as a voltage and/or current signal correlating to the received infrared light. In some embodiments, IR detector 510 produces a differential signal, such as a voltage or current that correlates to a change in infrared light received, such as an infrared sensor manufactured by Infrared Associates of Stuart Florida, such as a sensor similar to Infrared Associates model number MCT-12-0.25SC. IR detector 510 can be configured with a broad spectral response and a high efficiency for converting infrared light into the electrical signal. In some embodiments, the sensitivity or other performance characteristic of IR detector 510 is related to the area of detector 510.

Sensor assembly 500 can comprise a cooling assembly, not shown but such as a liquid nitrogen filled dewar, a thermoelectric cooler, a Stirling cycle cooler, or another refrigeration and/or cooling assembly constructed and arranged to maintain one or more components of sensor assembly 500 at a temperature below room temperature, such as to improve the sensitivity, accuracy, noise characteristics or response time of sensor assembly 500.

SPU 400 receives electrical or other signals from sensor assembly 500 via a single or multi-conductor cable, conductor 401. Alternatively or additionally, SPU 400 can receive electrical or other signals from sensor assembly 500 via wireless communication means such as Bluetooth. SPU 400 includes mechanical components, electrical components (e.g. one or more microprocessors; memory storage devices; analog circuitry such as analog filters or amplifiers; digital circuitry such as digital logic; and the like) and/or software (e.g. software including one or more signal processing algorithms, software configured to drive user interface 300, and the like) sufficient to perform one or more signal processing tasks on the signals received from sensor assembly 500.

SPU 400 can be configured to produce a video signal which is transmitted to user interface 300 via a single or multiple conductor cable, conductor 402. Alternatively or additionally, SPU 400 can transmit a video signal to user interface 300 via wireless communication means such as Bluetooth.

User interface 300 includes monitor 310 which can comprise at least one touch-screen or other visual display monitor. User interface 300 can include input device 320, which can include a component configured to allow an operator of system 10 to enter commands or other information into system 10, such as an input device selected from the group consisting of: monitor 310 such as when monitor 310 is a touch screen monitor; a keyboard; a mouse; a joystick; and combinations of these.

In some embodiments, command signals provided by user interface 300, such as via input device 320, can be transmitted to SPU 400 via conductor 402. The command signals can be used to command and/or configure (e.g. calibrate) SPU 400, sensor assembly 500 (e.g. via conductor 401). In some embodiments, the command signals from user interface 300 are received by SPU 400 and transmitted to motion transfer assembly 600 via a single or multiple conductor cable, conductor 403. In these embodiments, one or more rotation and/or translation parameters can be adjusted by an operator of system 10, such as a parameter selected from the group consisting of: translation travel (e.g. axial distance); translation speed; rotational travel (e.g. portion of circumferential travel such as 360° or less than 360°); rotational speed; scanning pattern geometry; position or range of positions of collector 220 within window 115; and combinations of these.

As described above, SPU 400 can create a table of values correlating measured temperatures to one or more collection locations proximate window 115 of probe 100. The tabularized information can be represented in alphanumeric form on monitor 310 of user interface 300. Alternatively or additionally, the tabularized information can be represented in the form of a graphical temperature map correlating the series of tissue locations to a two-dimensional representation of the cumulative tissue location geometry. The graphical temperature map can correlate colors, hues, contrast and/or other graphical parameters to represent an array of temperatures. In some embodiments, the correlation between the temperature and the visualizable parameter is adjustable by an operator of the system, such as a temperature map including a range of colors wherein the color correlation can be adjusted (e.g. a threshold is adjusted to set a particular temperature to a color). In addition to displaying a temperature map, additional temperature information can be provided by SPU 400 and user interface 300, such as numeric values for peak temperature or an average temperature of the entire set of collection locations or other statistical representations of the entire set or subset of collection locations or for two or more subsets of collection locations such as operator definable subsets of collection locations.

SPU 400 can include one or more algorithms (e.g. programs stored in memory of SPU 400) used to process (e.g. mathematically process) the signals received from sensor assembly 500 or further process an already processed signal. In some embodiments, an algorithm is included to perform a function selected from the group consisting of: averaging one or more values such as temperature values; finding the peak value of one or more temperature values; comparing peak values of one or more tissue areas; rate of change of tissue temperature; spatial rate of change, for example angular or linear rate of change, of tissue temperature, rate of rate of change of tissue temperature; determining an outlier value; and combinations of these. In some embodiments, an algorithm is included to determine an area of tissue whose average temperature is higher, or lower, than other areas measured.

In some embodiments, shaft 110 includes one or more functional elements, such as proximal band 125a and distal band 125b (generally band 125), which can be placed over and/or adjacent to the proximal and distal ends of window 115. Bands 125 can comprise a material selected from the group consisting of: a radiopaque material; aluminum, titanium, gold, copper, steel, iridium, platinum cobalt, chromium; and combinations of these. Bands 125 can be constructed and arranged such that when collector 220 is positioned within a band 125 (e.g. collects infrared light transmitted from band 125), a signal is received by sensor assembly 500 comprising a pre-determined or otherwise separately measurable signal, such as a pre-determined pattern of infrared reflectance or emissivity, or a measurable temperature.

In some embodiments, one or more bands 125 comprise one or more temperature sensors, such as a thermocouple or a thermistor, not shown but such as temperature sensor 121 of FIG. 2 herebelow and connected to one or more electrical wires or other information transfer conduits which transmit the temperature sensor information to sensor assembly 500 and/or SPU 400. In these embodiments, the temperature reading received from a band 125 can be correlated to the infrared light collected at that location by collector 220, such as to perform a calibration procedure of system 10. In some embodiments, a calibration procedure is performed at least once for each set of forward and back reciprocating translations (e.g. when collector 220 is within proximal band 125a or within distal band 125b). In other embodiments, a calibration procedure is performed at least twice for each set of forward and back reciprocating translations (e.g. when collector 220 is within proximal band 125a and when collector 220 is within distal band 125b).

One or more bands 125 or another component of probe 100 can be configured as a visualization marker, such as a marker selected from the group consisting of: a radiopaque marker such as a radiopaque marker band; an ultrasonically reflective marker; a visible light marker; a magnetic marker; and combinations of these. Bands 125 or other visualization markers of probe 100 can be used by a clinician to advance, retract, rotate or otherwise position probe 100 in relation to a body structure such as placement using fluoroscopy or ultrasound to position window 115 proximate the heart when probe 100 is placed into the esophagus (as described in reference to FIG. 2 herebelow).

In some embodiments, probe 100 comprises an functional element constructed and arranged to position a distal portion of probe 100 (e.g. window 115) relative to tissue, such as positioning element 118, shown in a deployed, radially expanded state in FIG. 1. Positioning element 118 can be constructed and arranged to be radially expanded and/or radially contracted. In some embodiments, positioning element 118 is constructed and arranged to position probe 100 in a body lumen, such as a balloon, an expandable cage, an expandable stent, and/or radially deployable arms constructed and arranged to center window 115 in a body lumen, such as the esophagus. Positioning element 118 can be constructed and arranged to position one or more portions of probe 100 towards and/or away from tissue. In some embodiments, probe 100 and/or positioning element 118 is constructed and arranged as described in applicant's co-pending International Patent Application Serial Number PCT/US2011/061802, entitled “Ablation and Temperature Measurement Devices”, filed Nov. 22, 2011, the contents of which is incorporated by reference in its entirety.

Referring now to FIG. 2, the distal end of the temperature measurement probe of FIG. 1 is illustrated, positioned in an esophagus and positioned near a heart chamber, consistent with the present inventive concepts. Probe 100 can be attached to one or more assemblies of system 10 described in reference to FIG. 1. Probe 100 includes shaft 110 and fiber assembly 200 including fiber 210 and an optical assembly comprising collector 220. Shaft 110 includes window 115 comprising one or more materials with high transmissivity to the desired wavelengths of infrared light. Positioned at each end of window 115 are proximal band 125a and distal band 125b (generally 125). Bands 125 can include one or more temperature sensors, such as one or more thermocouples, thermisters, or other temperature sensors. In the illustrated embodiment, thermocouple 121 is positioned on band 125a and is configured to measure temperature information of band 125a proximate one or more tissue T locations. Bands 125 can be positioned within a wall of shaft 110; on an outer surface of shaft 110 such as around an outer circumference of shaft 110, and/or on an inner surface of shaft 110 such as around an inner circumference of shaft 110. Bands 125 can comprise an infrared-opaque material and/or a material with a known emissivity, such that fiber assembly 200 records the infrared temperature information of bands 125 when infrared light emitted from a band 125 is received by collector 220. Bands 125 can comprise a radiopaque material such that bands 125 are visible to a visualization instrument so as to position distal end 112 of shaft 110, for example at a location within the esophagus most proximate a patient's heart. Examples of visualization instruments include: an MRI; a CT scanner; a fluoroscope or other x-ray instrument; and combinations of these.

Thermocouple 121 records temperature information, such as temperature dependent voltage information received by sensor assembly 500 and/or a signal processor, such as signal processor 400 of FIG. 1, via conduit 122, comprising one or more wires or other signal carrying conduits. Thermocouple 121 can be positioned within band 125a; on an outer surface of band 125a; on an inner surface of band 125a; and/or within a lumen of shaft 110. In some embodiments, thermocouple 121 is positioned within a lumen of shaft 110, and band 125a is positioned on an outer surface of shaft 110 such that band 125a surrounds shaft 110 and thermocouple 121.

In some embodiments, probe 100 can be used to monitor the temperature of the surface of the esophagus, such as during a clinical procedure where thermal application therapies (e.g. those using ablative heat or cold) are applied to the posterior wall of the heart. In some embodiments, probe 100 is inserted into the esophagus over a guidewire (e.g. over-the-wire insertion into a body lumen) and the guidewire is removed or partially withdrawn prior to performing one or more temperature measurements, such as to remove the guidewire from proximity to window 115. Thermal application therapies can include ablation therapies, such as RF ablation therapies performed using an ablation catheter, such as ablation catheter 20 comprising tip electrode 21. Thermal application therapies can also include but are not limited to therapies selected from the group consisting of: multiple electrode RF treatment; cryogenic treatment; laser energy treatment; ultrasound energy treatment; microwave energy treatment; and combinations of these. In the embodiment of FIG. 2, probe 100 is shown positioned such that optical viewing window 115 (the space between bands 125) is relatively centered with respect to electrode 21 of ablation catheter 20. Bands 125 can be visualizable such as to aid in positioning probe 100 in such a manner, for example under fluoroscopy when bands 125 comprise at least a radiopaque portion.

Referring now to FIG. 2A, a magnified sectional side view of the distal portion of the temperature measurement probe of FIG. 2 is illustrated, including an infrared light collector and consistent with the present inventive concepts. Probe 100 includes fiber assembly 200. Fiber assembly 200 includes fiber 210 and an optical assembly configured to collect infrared light, collector 220 positioned distal to fiber 210 as shown. Infrared light collected by collector 220 is focused onto distal face 214 of fiber 210. Fiber assembly 200 is configured to be rotated and/or translated within shaft 110, such as by rotating assembly 660 and/or translating assembly 610 described in reference to FIG. 1 hereabove. Optical fiber 210 can comprise a core diameter of between 6 and 1000 microns, such as a fiber with a diameter between 200 microns and 400 microns. Fiber 210 material can include a material configured to optimally transmit (e.g. provide minimal impedance to) infrared light in the 6-15 micron wavelength range, for example in the 8-11 micron wavelength range. In some embodiments, fiber 210 comprises a polycrystalline material such silver halide or one or more other materials with high transmissivity to a desired range of wavelengths of infrared light, such as a material selected from the group consisting of: zinc selenide; germanium; germanium oxide; chalcongenide; a hollow core material; and combinations of these. Optical fiber 210 can include a cladding layer which can be constructed and arranged to cause and/or maintain total internal reflection in the core of fiber 210 to ensure efficient transmission of the collected infrared light from the distal to proximal end of fiber 210. In some embodiments, fiber 210 does not include a cladding layer, instead an air envelope is positioned around the fiber to cause and/or maintain total internal reflection.

Fiber assembly 200 further includes sleeve 206, flange 207, torque shaft 205, and optical element 230. Sleeve 206, which surrounds the majority of the length of optical fiber 210, can be configured to protect optical fiber 210, for example by preventing direct contact between fiber 210 and torque shaft 205. Sleeve 206 can include infrared-opaque polymer tubing which can be configured to be non-reactive with fiber 210, for example when fiber 210 comprises a polycrystalline material. Probe 100 can include one or more components which contact fiber 210. These components can comprise a material configured to avoid damaging fiber 210, such as titanium, ceramic and/or polymer based components chosen to be non-reactive with a polycrystalline-based fiber 210. In some embodiments, another component of probe 100 can be polycrystalline-based, such as optical element 230, where its contacting components comprise a non-reactive material such as titanium, ceramic and/or a polymer.

In the embodiment of FIG. 2A, torque shaft 205 surrounds sleeve 206, flange 207 and optical fiber 210 along the length of fiber 210. Torque shaft 205 is configured to transmit rotational and translational forces from rotating and translating assemblies 660 and 610 respectively, from the proximal portion of fiber assembly 200, to collector 220 at the distal end of fiber assembly 200, such that fiber assembly 200, including collector 220 rotates and/or translates within shaft 110 as described herein. In some embodiments, torque shaft 205 comprises multiple wires or other filaments such as stainless steel or titanium wires. Shaft 205 can comprise multiple braided wires and/or multiple layers of wires wound in one or more directions (e.g. wound in opposite directions in two or more alternating layers). In some embodiments, up to 16 wires (e.g. 4 to 12 wires) are included in one or more layers of shaft 205.

Collector 220 can comprise structural and mechanical elements fabricated from a ceramic or titanium material, such as to prevent degradation of any polycrystalline-based components of collector 220, fiber 210 and/or another component of fiber assembly 200. Collector 220 includes proximal portion 222, including an opening, window 224. Collector 220 further includes distal portion 223, which includes an opening, window 229. Collector 220 includes a housing, housing 221 as shown. Torque shaft 205 and optical fiber 210 are attached to collector 220 at proximal portion 222. Flange 207 can surround the distal portion of fiber 210 and can include similar or dissimilar materials as sleeve 206. Flange 207 can be configured to geometrically center optical fiber 210 within window 224. In some embodiments, sleeve 206 and flange 207 can comprise a single component. A mid portion of collector 220 can include a gap, optical separation window 225, positioned between distal face 214 of fiber 210 and the opposing face of optical element 230. Optical separation window 225 facilitates focusing of infrared light from optical element 230 onto distal face 214 of optical fiber 210, as described in reference to FIG. 2B herebelow. Distal portion 223 of collector 220 houses optical element 230. Optical element 230 is surrounded by housing 226. Housing 226 can include a material similar or dissimilar to sleeve 206 and/or flange 207. Housing 226 comprises an opening, window 228, and can further comprise cap 227 configured to secure optical element 230 within housing 226, as well as rotationally align optical element 230 such as to be oriented towards window 228. Alternatively, optical element 230 can be fixed directly to distal portion 223 avoiding the need for housing 226.

Optical element 230 can include one or more components selected from the group consisting of: a lens; a mirror; a prism; and combinations of these. Optical element 230 can include similar or dissimilar materials to optical fiber 210. Optical element 230 can include one or more materials, such as a material configured to transmit (e.g. be relatively transparent to) infrared light and/or a material configured to reflect infrared light. In some embodiments, optical element 230 comprises an infrared transparent material attached to an infrared reflected material, such as is described in reference to optical element 230 of FIG. 2B herebelow.

Referring additionally to FIG. 2B, a perspective view of a segment of probe 100 of FIG. 2A is illustrated, including the pathway of collected infrared light and consistent with the present inventive concepts. In FIG. 2B, fiber 210 and optical assembly 250, including optical element 230, are shown, with other components of probe 100 removed for illustrative clarity. Optical element 230 includes planar surface 231, angled surface 232, and convex surface 233. In some embodiments, planar surface 231 can comprise a convex or concave geometry. Probe 100 is configured to collect and focus IR light 40 emanating from a surface of tissue area, tissue area TA, onto distal face 214 of optical fiber 210. First optical separation distance OS1 comprises the distance between tissue area TA and planar surface 231 of optical element 230. Second optical separation distance OS2 comprises the distance between distal face 214 of optical fiber 210 and convex surface 233 of optical element 230. Distance OS2 is determined based on the focusing requirements of optical element 230 and desired optical resolution of optical assembly 250, and is maintained by the geometry of collector 220 (e.g. the geometry of window 225 shown in FIG. 2A).

In the embodiments of FIGS. 2B, 3 and 4, fiber 210 and optical element 230 are constructed and arranged to define an optical assembly 250. In some embodiments, IR light 40 collected by optical assembly 250 from tissue area TA represents the infrared light collected from the conical projection of optical element 230 from planar surface 231 onto the surface of tissue area TA. IR light 40 collected from tissue area TA that is within the conical projection travels distance OS1 towards planar surface 231 of optical element 230. In other embodiments, a collimated or nearly collimated projection, a projection with a long beam-waist, and/or other geometric projection represents the collected infrared light. IR light 40 travels through optical element 230 towards angled surface 232, and is then reflected towards surface 233. IR light 40 is then focused by surface 233 onto the distal face 214 of optical fiber 210. Planar surface 231 can comprise a flat, convex, concave, curved, and/or an irregularly shaped surface configured to collect IR light 40 emitted from a surface of tissue area. Planar surface 231 can include a polished surface and/or it can include an anti-reflective coating as described above in reference to FIG. 1.

IR light 40 emitted from tissue area TA is collected by optical element 230 at surface 231, and travels through optical element 230 towards angled surface 232. Angled surface 232 can include a 45° angle, and can be coated, for example with a reflective coating such as a protected aluminum (PAL) or gold coating. Angled surface 232 can be configured to reflect IR light 40 perpendicularly towards convex surface 233 of optical element 230. In some embodiments, angled surface 232 can comprise an angle greater than or less than 45°, such as to meet optical requirements of optical assembly 250.

Infrared light reflected from angled surface 232 is reflected toward convex surface 233. Convex surface 233 is configured to focus IR light 40 onto the distal face 214 of optical fiber 210. Surface 233 can be coated with an anti-reflective coating, such as an anti-reflective coating similar or dissimilar to the anti-reflective coating of planar surface 231. In some embodiments, surface 231 can be flat, concave or include an irregularly shaped surface, such as to meet the optical requirements of optical assembly 250.

Optical assembly 250 comprises a numerical aperture comprising the range of angles over which infrared light is collected from a surface. Optical assembly 250's numerical aperture (NA) is the sine of, and therefore describes, the angle of the steepest light ray entering optical assembly 250 from tissue area TA and passing through fiber distal end 212. Since the NA is defined as the sine of this angle, the angle of the steepest ray increases as numerical aperture increases. The amount of IR light 40 collected from a particular point within a tissue area increases as the numerical aperture of optical assembly 250 increases. Generally, as the amount of IR light 40 collected increases (e.g. a higher NA), optical assembly 250 signal to noise ratio improves. Fiber 210 comprises an inherent maximum acceptance numerical aperture determined by the material of the core and cladding of fiber 210, specifically the index of refraction of these materials. IR light 40 entering fiber 210 at angles greater than the fiber 210's maximum numerical aperture will not be transmitted by fiber 210 to the sensor assembly 500. In some embodiments, fiber 210 comprises a maximum numerical aperture of 0.28 and a core diameter of 400 microns. In these embodiments, optical assembly 250 outputs a numerical aperture ranging from 0 to 0.28, for example, 0.11 to 0.14, thereby underfilling the maximum numerical aperture of the fiber.

An average temperature can be calculated for the tissue area TA based on the amount of IR light 40 which has been collected. In applications where this average temperature is to be displayed, or otherwise presented as a temperature versus two-dimensional location map (i.e. a map of multiple tissue locations), the area of each conical projection of optical assembly 250 is used to create this map and must be known or otherwise estimated. In some embodiments, distance OS1 to each measured tissue area TA can have minimal variance, and in other embodiments distance OS1 to each measured tissue area TA can have larger fluctuations. Provided herebelow in FIG. 3, optical assembly 250a is constructed and arranged to have a minimal depth of field, but higher optical assembly 250a Numerical Aperture and higher optical resolution for applications where the tissue surface distances are relatively uniform. Provided herebelow in FIG. 4, optical assembly 250b is constructed and arranged to have an extended depth of field, which correlates to a lower optical assembly 250b Numerical Aperture and lower optical resolution, such as for applications where the tissue surface distances are less uniform (greater variation in distances such as due to a non-uniform tissue surface).

Referring now to FIG. 3, an optical schematic of a “close-optimized” optical system is illustrated, including cross sectional representations of tissue surface areas and consistent with the present inventive concepts. The close-optimized optical assembly 250a is optimized to have a higher optical assembly 250a Numerical Aperture and higher optical resolution resulting in a smaller depth of field. These close-optimized embodiments are useful when the multiple tissue surface locations to be measured are known or likely to be within a limited range of distances from central axis A of optical element 230a. This optimization can be used to improve the accuracy and spatial resolution (e.g. pixel resolution) of a map of temperature versus tissue location, as is described in detail in reference to FIG. 1.

Optical assembly 250a, including optical element 230a, is constructed and arranged to have a small focal length near optical element 230a and/or a window surrounding optical element 230a as has been described hereabove. This short focal length results in a relatively short depth of field. In some embodiments, the focal length (e.g. a distance measured from the center axis of optical element 230a) can range from 1 to 10 mm, such as from 1 mm to 5 mm, such as approximately 3.2 mm and the associated tissue area from which infrared light is collected can range from 0.5 mm² to 1.5 mm². In visible light cameras, depth of field correlates to a range of distances in which a produced image appears acceptably sharp. In the temperature measurement systems and devices of the present invention, depth of field correlates to a range of distances around, before or beyond the focal length in which the tissue area from which infrared light collected is within an acceptable range of cross sectional areas, such as a range selected to meet a useful and acceptable spatial resolution criteria for temperature data collection. Depth of field varies depending on optical component 230a configuration, the numerical aperture of fiber 210, and distance OS2. In some embodiments, the depth of field can range from 0.1 to 15.0 mm, such as from 0.1 mm to 1.0 mm, such as a depth of field of approximately 0.5 mm around or beyond the optimal focal length. Optical element 230a focuses the collected infrared light 40 onto distal face 214 of optical fiber 210, such that the collected light can travel proximally to one or more sensor devices as described herein.

In some applications, tissue is positioned on or near the outer surface of an infrared transparent tube surrounding optical element 230a, such as window 115 of FIG. 1. Tissue in close proximity to the surrounding tube is often encountered in applications where the catheter is inserted into a body lumen having a diameter smaller than or relatively equivalent to that of the tube, for example when the body lumen includes the respiratory tract; colon; or urethra. In some embodiments, the body lumen can include the esophagus, for example when the system is used to monitor the temperature of the esophagus during cardiac ablation. The properties of the mammalian esophagus are such that the esophageal wall can collapse around the tube. In these embodiments, the focal length can be chosen to approximate the orthogonal distance between the central axis of optical element 230a and the outer surface of the surrounding tube, and the depth of field can be chosen to be small.

In optical assembly 250a, optical element 230a is configured, and optical separation distance (OS2) is selected, such that the focal length of optical assembly 250a is a distance X1 from the center axis A of optical element 230a. At the focal length X1 of optical assembly 250a, a cross sectional view of area TA1 is shown and includes diameter Y1. Diameter Y2 of area TA2 is determined by the cone of infrared light collected from tissue at distance X2 as shown, such that area TA2 is significantly larger than area TA1. Optical assembly 250a can comprise spatial resolution criteria (e.g. spatial accuracy criteria) that defines a corresponding depth of field centered about focal length X1. In some embodiments, distance X2 is within the focal length of optical assembly 250a, such that tissue positioned at distances between X1 and X2 are accurately measured. In other embodiments, distance X2 is outside the depth of field, and accurate temperature measurements must be performed within the appropriate depth of field (i.e. at a threshold distance less than X2). In the embodiment of FIG. 3, tissue locations within optical assembly 250's resolution-based depth of field can have a cross sectional area approximately equal to area TA1, such as an area within 0.01 mm² of the area of TA1. For tissue locations positioned outside the depth of field, such as area TA2, as shown, the cross sectional area is larger than area TA1, such as comprising an area more than 1 mm² greater than the area of TA1. In one embodiment of optical assembly 250a, fiber optic 210 has approximately a 400 μm diameter core; distance OS2 comprises a length of approximately 4.5 mm; optical element 230a is fabricated of zinc selenide; and lens surface 233a has a convex radius of approximately 3 mm. In this particular embodiment, focal length X1 is approximately equal to 3.5 mm, and TA1 has a diameter Y1 of approximately 0.4 mm (e.g. area TA1 is approximately 0.13 mm²). Optical assembly 250a can include spatial resolution criteria such that an acceptable depth of field includes tissue positioned at distances out to 7.5 mm (i.e. tissue areas with a diameter greater than 1 mm). Alternatively, optical assembly 250a can include spatial resolution criteria such that an acceptable depth of field includes tissue areas with a diameter of approximately 1 mm (i.e. a depth of field that does not include tissue positioned at distance X2).

As described above, the close-optimized optical assembly 250a can be constructed and arranged to provide accurate temperature measurements for tissue areas at a relatively fixed distance from central axis A. Optical assembly 250a can include spatial resolution criteria (e.g. spatial accuracy criteria) that determines an acceptable depth of field from its focal length X1. Optical assembly 250a can be utilized if tissue to be measured would be primarily positioned near (e.g. close to or in contact with luminal wall tissue) the outer surface of an infrared transparent tube surrounding optical element 230a, such as window 115 of FIG. 1. Maximal resolution is achieved at tissue surfaces positioned at these close distances. Tissue positioned at greater distances, result in lower spatial accuracy.

Referring now to FIG. 4, an optical schematic of a “range-optimized” optical system is illustrated, including cross sectional representations of tissue surface areas and consistent with the present inventive concepts. The range-optimized optical assembly 250b is optimized to provide accurate temperature measurements for tissue surfaces positioned at greater distances and/or greater variation in distances (e.g. from central axis A) than the close-optimized optical assembly 250a of FIG. 3. For example, optical assembly 250b can be constructed and arranged to provide a consistent resolution over a larger depth of field and/or provide a more accurate resolution at greater distances from the system's focal length. Optical assembly 250b can be selected for use when the multiple tissue surface locations to be measured are known or likely to be within a wider range of distances from central axis A of optical element 230b. This optimization can be used to improve the accuracy of a map of temperature versus tissue location, as is described in detail in reference to FIG. 1. The trade-off for the range-optimized optical assembly 250b of FIG. 4 is that the minimum temperature measurement area is not as small as that in the close-optimized optical assembly 250a of FIG. 3. In other words, optical assembly 250b results in a reduction in spatial resolution at the focal length, however its spatial resolution is reasonably consistent over a much larger range of distances from the focal length.

Optical assembly 250b including optical element 230b is constructed and arranged to have a focal length X3 and a relatively long depth of field. In some embodiments, the depth of field can range from 1.5 mm and 10 mm, such as a depth of field of approximately 7 mm. Optical element 230b focuses the collected infrared light 40 onto distal face 214 of optical fiber 210, such that the collected light can travel proximally to one or more sensor devices as described herein.

In some applications, tissue is positioned away from, both close and away from and/or at unknown distances from an infrared transparent tube surrounding optical element 230b, such as window 115 of FIG. 1. Tissue positioned away and/or at varying distances from the surrounding tube can be encountered in larger body lumens such as the esophagus, colon, respiratory tract or the stomach. When positioned in a body lumen such as the esophagus, one or more distances between the tissue and the surrounding tube can be unknown. For these various applications, the focal length can be chosen to approximate the natural or relaxed radius of the body lumen, while the depth of field can be chosen to approximate the variation in the radius of the body lumen or variation in the position of the device within the body lumen (e.g. positioned in contact with a circumferential segment of a luminal wall while at a relatively large distance from an opposing circumferential segment of the wall). In some embodiments, the body lumen can include the esophagus, for example when the system is used to monitor the temperature of the esophagus during cardiac ablation and when the esophageal wall is assumed to be within a range of distance from the surrounding tube. For example, when positioned against one circumferential segment of the esophageal wall (e.g. 0 mm from the surrounding tube or approximately 1.5 mm from center axis A of optical element 230b), the apposing segment of the esophageal wall can be from 0 mm to 10 mm from the surrounding tube. In these embodiments, the optimal focal length can be chosen to approximate half the distance between the outer surface of the surrounding tube and the greatest assumed distance to the tissue surface (e.g. a focal length between 0 mm to 10 mm). The depth of field can be configured to approximate the range of distances assumed or expected to be encountered during the performance of a temperature measurement.

In optical assembly 250b, optical element 230b is configured, and optical separation distance OS2 is selected such that the focal length of optical assembly 250b is a distance X3 from the center axis A, of the optical element 230b. At the focal length X3 of optical assembly 250b, the diameter of tissue area TA3 is represented by diameter Y3. In some embodiments distance X3 (i.e. the focal length) can range from 4 mm to 10 mm, such as approximately 7 mm, and the diameter Y3 of tissue area TA3 can range from 0.5 mm² to 1.5 mm². A cross sectional view of the tissue area at a distance X4, area TA4, is shown in FIG. 4. Tissue locations within the depth of field of the optical assembly 250b have a cross sectional area approximately equal to area TA3, such as an area within 0.2 mm², within 0.1 mm², or within 0.01 mm² of the area of TA3. Optical assembly 250b has a long depth of field, such that tissue locations positioned an appropriate distance away from the focal length (as determined by optical assembly 250b's spatial resolution criteria), such as distance X4 as shown (or at a distance nearer to a surrounding tube, not shown), comprises a cross sectional area approximately equal to area TA3, such as comprising an area within 0.2 mm² of the area of TA3.

In one embodiment of optical assembly 250b, fiber optic 210 comprises an approximately 400 μm diameter core; distance OS2 comprises a length of approximately 4.2 mm; optical element 230b is comprised of zinc selenide; and lens surface 233b has a convex radius of approximately 4 mm. In this particular embodiment, the focal length X3 would be approximately equal to 7.5 mm, and TA3 would have a diameter Y3 of approximately 1.0 mm (i.e. area TA3 comprises an area of approximately 0.79 mm²). Optical assembly 250b can include spatial resolution criteria such that an acceptable depth of field includes tissue positioned within a maximum distance on either side of focal length X3, such as within 4 mm on either side of focal length of focal length X3 (e.g. a depth of field of 8 mm). The range-optimized embodiment of FIG. 4 can be selected if it was expected that the tissue to be measured might be positioned over a wide range of distances between the tissue and optical element 230b.

Referring now to FIGS. 5A and 5B, a perspective view and a perspective partial cross sectional view, respectively, of a sensor assembly and a rotating assembly is illustrated, consistent with the present inventive concepts. Rotating assembly 660 is operably connected to fiber assembly 200, such as has been described in detail with in reference to FIG. 1 hereabove.

Rotating assembly 660 includes motor 665 which is configured to rotate fiber assembly 200. Rotating assembly 660 can rotate fiber assembly 200 at a speed ranging from 1000 rpm to 15000 rpm, such as a speed between 4000 and 8000 rpm, such as a speed of approximately 7260 rpm. Each rotation can include a full 360° rotation or a partial rotation less than 360°, for example rotations up to 180° or up to 90°.

In some embodiments, rotating assembly 660 can be configured to rotate fiber assembly 200 with a frictionally engaged belt driven assembly as described herebelow. Various configurations can be used to rotate fiber assembly 200, such as an in-line or co-axial drive assembly; a magnetic field driven assembly; and combinations of these.

In the embodiment of FIGS. 5A and 5B, rotating assembly 660 includes housing 661 which is attached to and/or maintains the relative position of one or more components of rotating assembly 660. Housing 661 can be further attached and/or maintain the position of other components of system 10, such as sensor assembly 500 and/or a translating assembly such as assembly 610 described herein. Rotating assembly 660 further includes first pulley 666, belt 667, torque assembly 670, and second pulley 671. Pulley 671 is incorporated within torque assembly 670. Torque assembly 670 further includes bearings 672, set screw 673, rotational encoder 675, a rotational encoder wheel 676, and fiber assembly coupling 680. Coupling 680 frictionally or otherwise operably engages the proximal portion of fiber assembly 200, such as to transfer rotational forces to torque shaft 205 of fiber assembly 200. Coupling 680 can be attached to fiber assembly 200 via a press-fit, adhesive or the like.

Coupling 680 is attached to housing 661 via bearings 672. Bearings 672 maintain the position of coupling 680 within housing 661, while allowing coupling 680 to freely rotate about its center axis. Bearings 672 are configured to be coaxial with coupling 680 as well as fiber assembly 200. Pulley 671 is fixedly attached to coupling 680, such as to transfer rotational forces to coupling 680. Pulley 671 can be fixedly attached to coupling 680 via one or more of set screw 673, adhesive, or the like. Rotational encoder wheel 676 is fixedly attached to coupling 680 and/or pulley 671. Rotational encoder wheel 676 maintains its angular position and velocity such that it matches the angular position and velocity of fiber assembly 200, such that the position of wheel 676 can be determined by rotational encoder 675, and that information can be transmitted to a signal processor such as signal processor 400 of FIG. 1.

Motor 665 is fixedly attached to pulley 666, such that motor 665 rotates pulley 666, rotating drive belt 667, and further rotating pulley 671 which in turn rotates torque assembly 670. Rotating assembly 660 further includes an adjustment assembly, such as at least a two dimensional adjustment mechanism, such as X-Y table 690. X-Y table 690 can be configured to fixedly attach to housing 661, such as to position housing 661 in two dimensional space. Housing 661 is fixedly attached to torque assembly 670, such X-Y table 690 can align the proximal face of optical fiber 210 with the sensor assembly 500. X-Y table 690 includes first adjustment screw 691 and second adjustment screw 692, such that first adjustment screw 691 adjusts in a first dimension and second adjustment screw 692 adjusts in a second dimension orthogonal to the first direction. Adjustment screws 691 and 692 can be used to center the proximal face of optical fiber 210, such that infrared light is collected properly by the sensor assembly 500, as described in reference to FIG. 7 herebelow.

Referring now to FIG. 6, a perspective view of a translating assembly is illustrated, consistent with the present inventive concepts. Translating assembly 610 includes motor 615, drive screw 620, translating car 625, guide 628, and linear encoder 630. Motor 615 rotates drive screw 620 such that drive screw 620 translates car 625 proximally and distally. In the embodiment of FIG. 6, drive screw 620 comprises a Yankee screw, such as is commonly used as component of a line guide in a baitcasting fishing reel. This configuration allows relatively constant speed of car 625 when motor 615 rotates at a constant velocity, and in a single rotational direction. The gears internal to car 625 allow car 625 to translate to one end of drive screw 620, where the internal gears switch position, and car 625 translates the opposite direction, to the other end of screw 620, where the gears switch back to the original orientation. In this configuration, in addition to the linear speed of car 625 being relatively uniform, reversing direction at the end of each translation is achieved relatively instantaneously. In an alternative embodiment, drive screw 620 comprises a worm drive, and the direction of travel is dependent on the rotational direction of motor 615.

Guide element 628 guides car 625 linearly such that car 625 does not rotate about drive screw 620. Guide element 628 includes linear encoder 630 which is configured to determine the linear position of car 625, and to transmit positional information to a signal processor such as signal processor 400 of FIG. 1. Car 625 includes bearings 626 which are configured to fixedly attach coupling 627 to car 625 such that coupling 627 translates with car 625. Coupling 627 is configured to be fixedly attached to fiber assembly 200, such that coupling 627 transfers linear translational forces to fiber assembly 200. Additionally, coupling 627 rotates with fiber assembly 200 as fiber assembly 200 is rotated by rotating assembly 660 as described in reference to FIGS. 5A and 5B hereabove.

Translation assembly 610 further includes housings 611a, 611b, and 611c, (generally 611). Housings 611 are attached to and/or maintain the relative position of one or more components of translating assembly 610. Housings 611 can be further attached and/or maintain the position of other components of system 10, such as sensor assembly 500 or rotating assembly 660 described herein. Housing 611c is configured to fixedly attach to the proximal end of shaft 110 of probe 100, such that shaft 110 does not translate with respect to system 10, and fiber assembly 200 translates within a lumen of shaft 110.

Referring now to FIG. 7, an optical schematic of an optical assembly proximate a sensor assembly is illustrated, consistent with the present inventive concepts. In some embodiments, optical assembly 520 can be included in a sensor assembly, such as sensor assembly 500 described herein. Optical assembly 520 can include various optical components configured to focus, split, filter, transmit without filtering (e.g. pass through), amplify, refract, reflect, polarize, or otherwise handle light such as infrared light. Typical optical components include but are not limited to: optical fiber; lens; mirror; filter; prism; amplifier; refractor; splitter; polarizer; aperture; and combinations of these. Optical assembly 520 is configured to focus IR light 40, which is received from fiber 210 as has been described herein. Optical assembly 520 includes lens 521, optical window 522, filter 523, aperture 524, and immersion lens 526. Any or all components of assembly 520 can be housed within a housing, such as sensor detector housing 501. Detector housing 501 can be a cooled housing, such as a Stirling cooled housing. Components of optical assembly 520 can include similar or dissimilar materials to the materials of optical fiber 210, such as materials configured to pass (e.g. be relatively transparent to) infrared light in the 6-15 micron wavelength range, such as light in the 8-11 micron wavelength range, as has been described herein. One or more components of assembly 520 can include anti-reflective coatings, as described in reference to FIG. 1 hereabove.

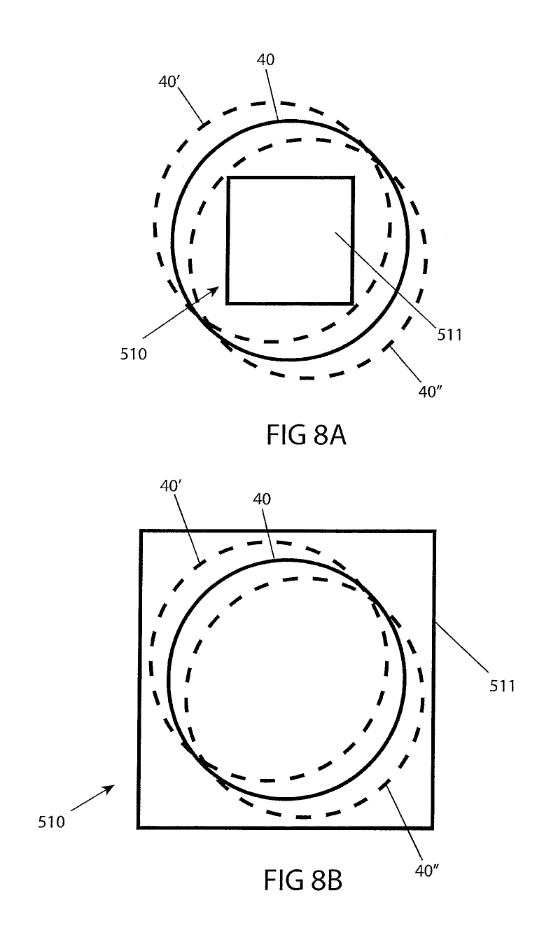

IR light 40 collected from a surface of a tissue area passes through focusing lens 521, which is configured to focus IR light 40 towards detector 510. Detector 510 includes a receiving surface, receiving surface 511, reference number not shown on FIG. 7 for clarity, but included on FIGS. 8A and 8B herebelow. Fiber 210 is separated from focusing lens 521 by a physical gap, distance D1. D1 can be varied, either during use or in a manufacturing process, such as to set the magnification of IR light 40 throughout optical assembly 520. IR light 40 then passes through optical window 522, an optical component which provides a seal of a detector housing 501 (e.g. a seal that enables deep cooling of components within detector housing 501). Optical window 522 can comprise an optical component such as a planar or wedged window, a filter, or a lens configured to allow IR light 40 to pass into detector housing 501. Some or all components of assembly 520 can be enclosed within detector housing 501, including IR detector 510, such as when detector housing 501 comprises a cooled housing and the enclosed components are cooled to the temperature in detector housing 501. Cooling of the components within detector housing 501 minimizes the amount of infrared light emitted by the components, and thus increasing the signal to noise ratio of the system. In some embodiments, components within detector housing 501 are cooled to approximately 77 degrees Kelvin. Filter 523 comprises an optical filter configured to pass IR light 40, for example infrared light comprising a wavelength of between 8 and 11 microns. All other wavelengths are blocked, or partially blocked by filter 523, increasing the signal to noise ratio of the system.

Assembly 520 further includes a cold aperture, aperture 524, configured to block infrared light from outside the desired field of view from reaching detector 510. Immersion lens 526 further focuses IR light 40 onto the face of detector 510. Immersion lens 526 allows optical assembly 520 to incorporate a smaller light-receiving surface of detector 510 without increasing the overall length of the system. Reducing the light receiving surface area of detector 510 can provide improved signal to noise and/or temporal performance (e.g. faster response time). Furthermore, the shape of detector 510 can optimally be matched to the core shape of fiber 210 (e.g. example round or square) such as to minimize the area of detector 510 that is unlikely to receive IR Light 40. IR light 40 emitted onto the face of detector 510 can be converted into a voltage signal, as described in reference to FIG. 1 hereabove, and converted to a table of temperature values versus tissue area locations, which can be displayed as a temperature map as described herein. In some embodiments, the voltage signal represents a change in IR light 40 received by detector 510 (i.e. a differential signal).

The optical pathway of FIG. 7 can be constructed and arranged to relatively fill, “overfill” or “underfill” the receiving surface of detector 510 with IR light 40, such as is described herebelow in reference to FIGS. 8A and 8B, respectively.

FIG. 8A is an optical schematic of an infrared detector illustrating the projections of infrared light focused toward the detector, in a configuration that overfills the detector, consistent with the present inventive concepts. Detector 510 can be similar to detector 510 of FIG. 7 described hereabove, and includes a receiving surface 511 constructed and arranged to receive infrared light such that detector 510 can convert the received infrared light into a signal. IR light 40 can represent the projection of infrared light focused towards detector 510, such as by an optical assembly, such as optical assembly 520 of FIG. 7. In the illustrated embodiment, detector 510 is “overfilled”, such that projections of received infrared light (e.g. light received from fiber 210 of FIG. 7) fully cover, and potentially extend beyond, the receiving surface 511 of detector 510. IR light 40, 40′ and 40″ represent projections that have a cross sectional area greater than surface 511 (e.g. due to the magnification that results from optical assembly 520 of FIG. 7). IR light 40 is relatively centered about surface 511. IR light 40′ and 40″ can represent a precession of light 40 away from its centered position, such as a precession caused by one or more of: static alignment or misalignment of one or more optical components; irregular rotation of an optical fiber or other rotating component of the system; or another cause.

FIG. 8B is an optical schematic of an infrared detector illustrating the projections of infrared light focused toward the detector, in a configuration that underfills the detector, consistent with the present inventive concepts. Detector 510 can be similar to detector 510 of FIG. 7 described hereabove. IR light 40 can represent the projection of infrared light focused towards detector 510 (e.g. from a fiber such as fiber 210 of FIG. 7), such as by an optical assembly, such as optical assembly 520 of FIG. 7. In the illustrated embodiment, detector 510 is “underfilled”, such that projections of received infrared light partially cover the receiving surface 511 of detector 510. IR light 40, 40′ and 40″ represent projections have a cross sectional area less than surface 511 (e.g. due to the magnification that results optical assembly 520 of FIG. 7). IR light 40 is relatively centered about surface 511. IR light 40′ and 40″ can represent a precession of IR light 40 away from its centered position, such as a precession caused by one or more of: static alignment or misalignment of one or more optical components; irregular rotation of an optical fiber or other rotating component of the system; or another cause. In some embodiments, a proximal optical assembly (e.g. optical assembly 520 of FIG. 7) can be constructed and arranged such that all anticipated precessions of IR light 40 (e.g. IR light 40′ and 40″) are fully received by (e.g. do not extent beyond) surface 511.

In some embodiments, the overfill design of FIG. 8A is selected, such as to minimize infrared light emanating from objects or surfaces other than the proximal end of fiber 210 from being received by surface 511; minimize errors that result from misalignment, non-uniform rotation or other abnormalities that can cause light emanating from the proximal end of fiber 210 to move onto and/or off of surface 511; and combinations thereof. In other embodiments, the underfill design of FIG. 8B is selected, such as to maximize the amount of light received by surface 511 that emanates from the proximal end of fiber 210. In some embodiments, optical assembly 520 is constructed and arranged to relatively, completely “fill” detector 510, such that the size of the projected light onto surface 511 relatively matches the size of surface 511. In some embodiment, the system of the present inventive concepts is constructed and arranged to allow an operator to change the amount of fill or overfill of infrared light received on surface 511, such as by adjusting the magnification of optical assembly 520 as has been described hereabove.

In the embodiments of FIGS. 8A and 8B, receiving surface 511 of detector 510 comprises a square infrared light receiving surface. In other embodiments, surface 511 can comprise a surface with a shape selected from the group consisting of: circular; elliptical; rectangular; trapezoidal; triangular; and combinations of these. In some embodiments, receiving surface 511 comprises a shape configured to match an optical component of the system, such as the cross sectional shape of an optical fiber (e.g. optical fiber 210 of FIG. 7), or the shape of the projection of infrared light from a lens (e.g. focusing lens 521 of FIG. 7). In some embodiments, optical assembly 520 is constructed and arranged to project IR light 40 onto surface 511 in a circular, elliptical, rectangular or square pattern, such as when surface 511 comprises a circular, elliptical, rectangular or square pattern, respectively.

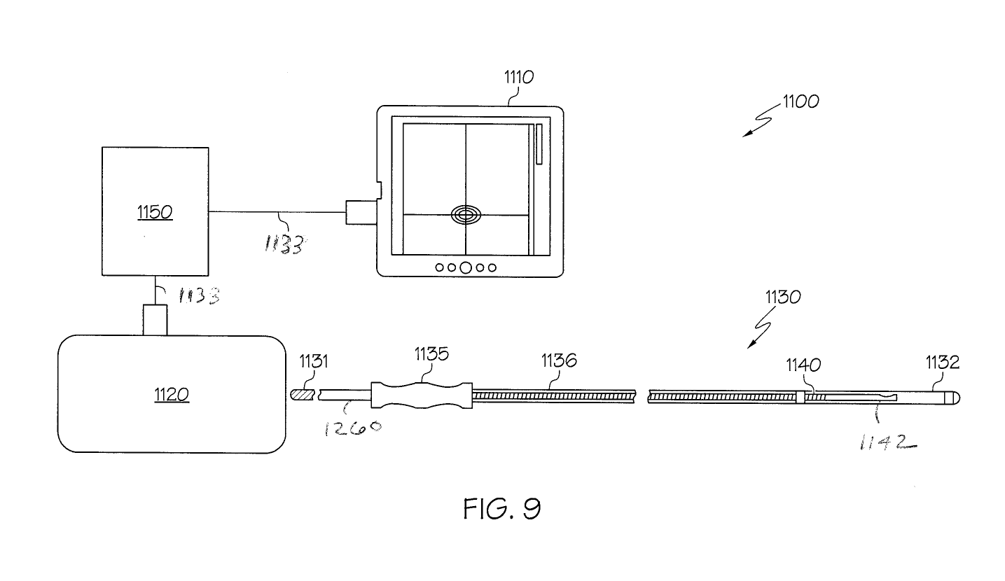

FIG. 9 is a schematic view of a temperature measurement system 1100, consistent with the present inventive concepts.

In the present embodiment, system 1100 comprises monitoring unit 1110, patient interface unit 1120, probe assembly 1130, and processor 1150. Monitoring unit 1110, patient interface unit 1120, and processor 1150 can include a connector for coupling the respective unit to an individual or common power source. In some embodiments, probe assembly 1130 can include passive elements such as an optical fiber for receiving IR energy or the like, for example, as described herein. In some embodiments, monitoring unit 1110, patient interface unit 1120, and processor 1150 are physically separate devices, which can communicate with each other by a communication connector 1133, such as an Ethernet cable, wireless interface, and so on. In other embodiments, some or all of the monitoring unit 1110, patient interface unit 1120, and/or processor 1150 can be combined under a single platform. Probe assembly 1130 and patient interface unit 1120 can communicate via wired or wireless communications when the probe 1130 is inserted at the patient interface unit 1120. Wireless communications can include but not be limited to Radio Frequency identification (RFID) tagging, Bluetooth or Bluetooth Low Energy (BTLE), 1-Wire, or other wireless technologies. Wired communications can be accomplished with small electrical connectors that could be incorporated into the coupling design to allow information stored on the probe 1130, for example, described herein, to be transmitted to the patient interface unit 1120. Data related to usage tracking, calibration values, serial number, security information, customer identification, and so on can be stored at the system or an external storage device, processed by the system, and/or exchanged between the probe 1130 and the patient interface unit 1120, for example, during an operation.

Monitoring unit 1110 can have a user interface that is similar to, or the same as, user interface 300 referred to herein. Processor 1150 can be similar to, or the same as, SPU 400 referred to herein. In particular, processor 1150 can be configured to convert the infrared energy received at the at least one fiber into a plurality of temperature measurements. Monitoring unit 1110 can include an input device allowing an operator to enter data such as commands or other information to system 1100, and a user interface. Accordingly, monitoring unit 1110 can receive and display at the user interface temperature information, for example, displayed as a temperature map, temperature values, present temperature information, past temperature information, and so on, in response to IR energy received at a body lumen wall or related tissue surface from probe assembly 1130.

Probe assembly 1130 includes a proximal end 1131 and a distal end 1132. In some embodiments, probe assembly 1130 can include a handle 1135 near or at the proximal end, which can be inserted by an operator, or automatically coupled, to patient interface unit 1120 according to one or more different coupling configurations described in embodiments herein.

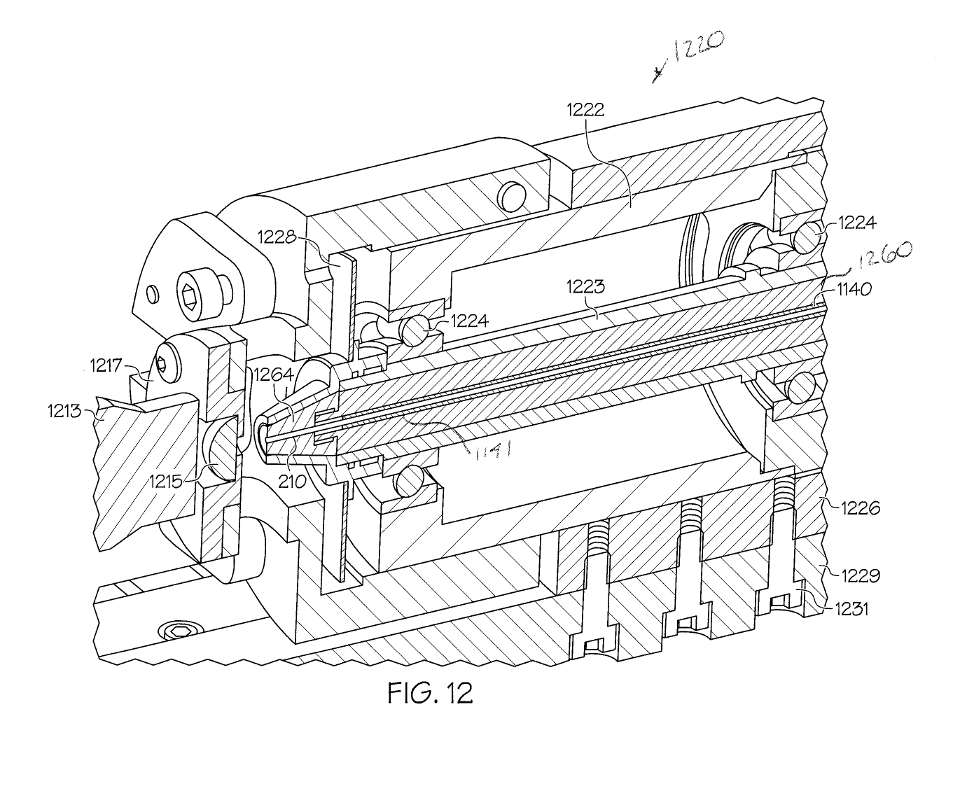

In some embodiments, probe assembly 1130 can include a sheath 1136 coupled to handle 1135. A fiber assembly 1140 comprising at least one optical fiber or related infrared signal transport element can be positioned in sheath 1136, or hypotube or the like, and extend from the proximal end 1131 through the handle 1135 and sheath 1136, to the distal end 1132. Accordingly, fiber assembly 1140 positioned in probe assembly 1130 can receive IR energy at or near its distal end 1142, and channel or otherwise direct the received IR energy down its length to its proximal end 1141 (see FIG. 12) coupled at the patient interface unit 1120. In particular, a distal end of an optical fiber of probe assembly 1130 can receive IR energy or the like from one or more tissue surface locations at or proximal to the body lumen in which probe assembly 1130 is inserted, for example, in a similar or same manner as other embodiments described herein. The collected IR energy can be transmitted along the fiber or fibers of the fiber assembly 1130 to the proximal end 1141. The optical fibers in the fiber assembly 1140 can be the same as or similar to fibers of other embodiments herein. Other optical elements such as mirrors, reflectors, sensors, and so on can also be included in probe assembly 1130, for example, as described in other embodiments herein. In other embodiments, temperature sensors (not shown), such as a thermocouple or a thermistor, for example, of the type of temperature sensor 121 of FIG. 2 herein, can be included in probe assembly 1130 for collecting reference temperature data or the like, and can be connected to one or more electrical wires or other information transfer conduits which transmit the temperature sensor information to sensor assembly patient interface unit 1120 and/or monitoring unit 1110.

In some embodiments, probe assembly 1130 can be constructed and arranged as a sterile, single-use or multi-use, catheter, which is inserted into a body lumen such as an esophagus, respiratory tract or colon, or other body lumen for performing thermal imaging or related operation. In some embodiments, the probe assembly can be positioned using a corresponding guide wire, as described herein. In some embodiments, probe assembly 1130 includes some elements that can be reusable. For example, in some embodiments the fiber assembly 1140 can be reused, while portions that may come in contact with the patient during a procedure such as handle 1135 and sheath 1136 can be disposable. In other embodiments, the fiber assembly 1140 can be disposable as well.

In some embodiments, patient interface unit 1120 or the proximal end 1131 of probe assembly 1130 can include a sensor (not shown in FIG. 9) that receives and processes received IR energy, or related signals, for example, collected from a particular tissue region, and determines temperature-related data from the received IR energy, such as an average temperature of the tissue surface area, a rate of change of temperature, or a two-dimensional temperature map of the region. In some embodiments, the rate of change of temperature in time or space can be determined in an angular or axial direction. The sensor can be the same as or similar to other sensors described herein. For example, the sensor can comprise an IR light detector in communication with the proximal end of the fiber assembly 1140.

In some embodiments, the sheath 1136 of the probe assembly 1130 can comprise a plastic tube that covers the spinning and/or translating fiber assembly 1140, thereby protecting the patient from frictional trauma. In various embodiments, the fiber assembly 1140 can be integral with or removable from the sheath 1136.

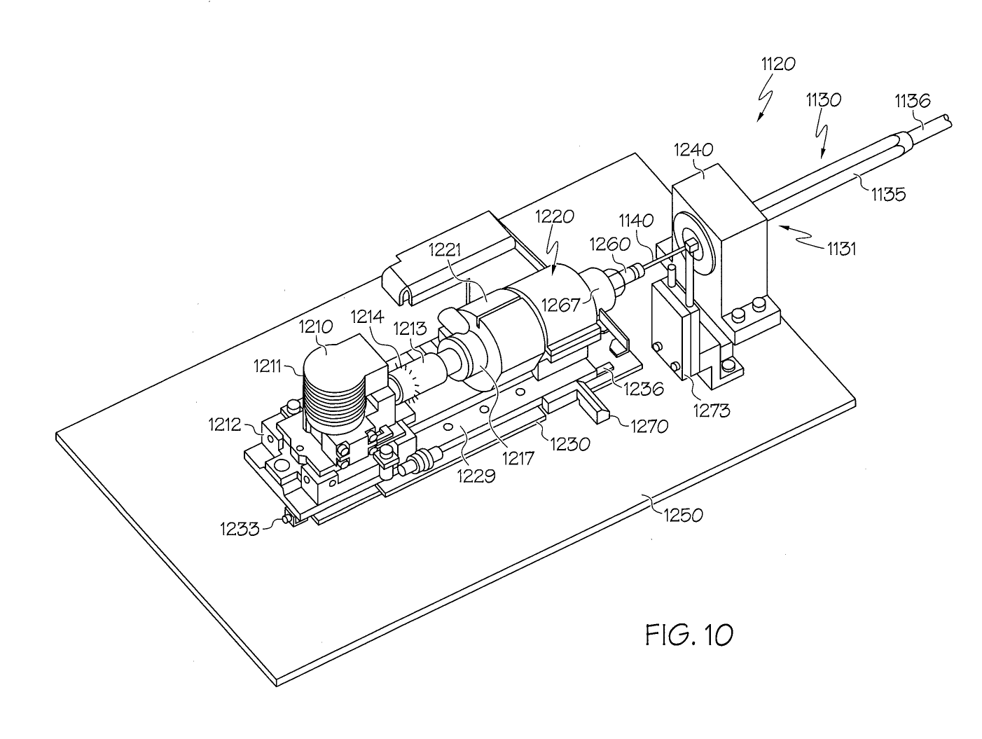

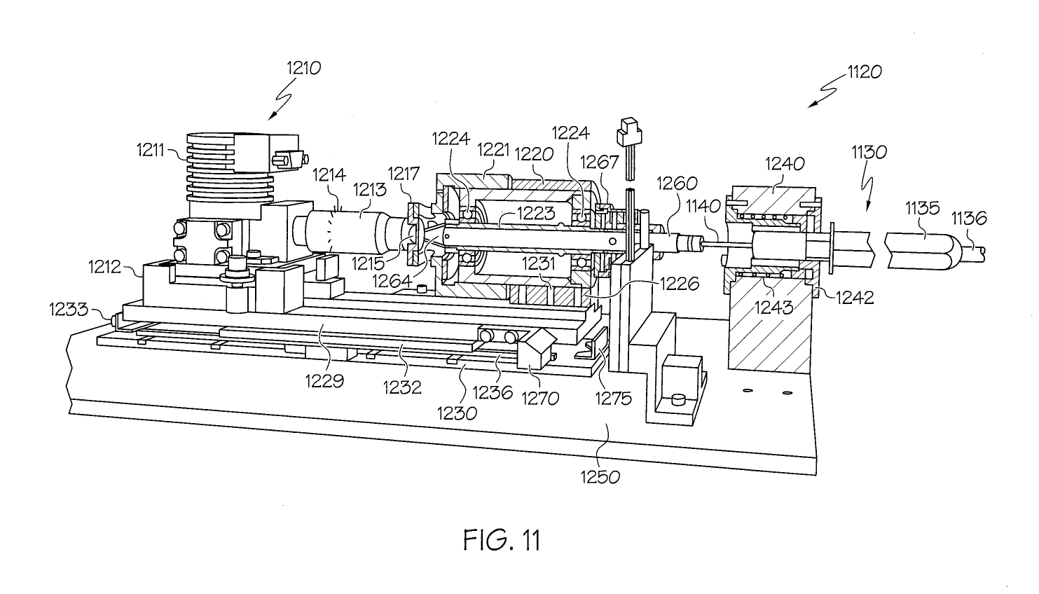

FIG. 10 is a perspective view of an embodiment of the patient interface unit 1120 of FIG. 9, consistent with the present inventive concepts. FIG. 11 is a perspective partial cross-sectional view of elements of patient interface unit 1120 of FIGS. 9 and 10, consistent with the present inventive concepts.

Referring now to FIGS. 10 and 11, in some embodiments, patient interface unit 1120, also referred to as a motion unit, comprises sensor assembly 1210 and rotary motor assembly 1220, at least one of which can be positioned on a translation table 1229. Translation table 1229, in turn, can be moved linearly by a linear translation motor assembly 1230 positioned between base table 1250 and translation table 1229. In this manner, sensor assembly 1210 and rotary motor assembly 1220 can translate in the linear direction along with translation table 1229, as driven by linear translation motor assembly 1230. In other embodiments, sensor assembly 1210 can be positioned at a location other than the translation table 1229 and/or base 1250. In such an alternative embodiment, the sensor assembly 1210 does not undergo the linear translation, but rather, remains fixed relative to the base 1250.

Linear translation motor assembly 1230 includes a carriage 1232 for moving the translation table 1229 along a direction of linear extension. In various embodiments, the linear translation motor assembly 1230 can provide motion along stationary rails 1236, and/or a platform, a magnetic system, and/or other linear path (not shown). In some embodiments, a proximal end 1141 of the fiber assembly 1140 is likewise mounted to a component mounted to the translation table 1229, for example, mounted to rotary motor assembly 1220. As such, the fiber assembly 1140 can therefore be caused to travel, or translate, in a linear direction relative to stationary base 1250. Rotational motion in the fiber assembly 1140 is induced by rotary motor assembly 1220.

As discussed, in the present embodiment, the rotary motor assembly 1220 is positioned on the translation table 1229 controlled by linear translation motor assembly 1230. This configuration reduces or eliminates the need for cabling or other connectors between motors 1220, 1230, thereby reducing the system footprint and configuration complexities otherwise found when such motors are disparate with respect to each other. The configuration of the IR detector, proximal optics and rotational motor mounted on the translation table also reduces or eliminates the need for providing a service loop of fiber optic between rotary motor 1220 and linear motor 1230 as distinguished from the embodiment of FIG. 1.

Linear translation motor assembly 1230 can be computer-controlled, for example, in response to a user issuing commands from monitoring unit 1110 and/or processor 1150. A power connector 1233 can receive and provide power to one or more elements of patient interface unit 1120 such as rotary motor assembly 1220, linear translation motor assembly 1230, and/or sensor assembly 1210.

Patient interface unit 1120 includes a sheath attachment frame 1240 directly coupled to base table 1250 or alternatively, positioned on a different movable table than translation table 1229 on which linear translation motor assembly 1230 is positioned. Sheath attachment frame 1240 is constructed and arranged to receive the handle 1135 of probe assembly 1130 and maintain handle 1135 in a stationary position, permitting a probe connector 1260 of proximal end 1131 of probe assembly 1130 to uncouple from handle 1135 during operation, for example, as shown and described herein in connection with the embodiment of FIG. 11, and thereby permitting fiber assembly 1140 to translate and/or rotate relative to probe assembly 1130, as described in detail herein.

Patient interface unit 1120 can include one or more wire holders (not shown) for housing or otherwise managing the location of various wires extending from other components of the patient interface unit 1120 such as sensor assembly 1210, rotary motor 1220, and or linear motor 1130. The wire holders can be constructed and arranged to prevent the wires from interfering, for example, tangling, when rotational and/or linear movements occur during operation, for example, during a rotation of fiber assembly 1140 between sheath attachment frame 1240 and rotary motor 1220.

Sensor assembly 1210 processes signals related to IR energy or the like emitted from multiple tissue surface areas and received from probe assembly 1130, for example, in a manner similar to the sensor assembly 500 referred to herein. A description of some features common to both the sensor assembly 1210 and sensor assembly 500 are not repeated for brevity. Sensor assembly 1210 can convert the received IR energy into one or more information signals related to temperature data corresponding to the IR energy collected from the target tissue area and transmit the information signals to processor 1150 and/or monitoring unit 1110.

Sensor assembly 1210 can comprise a cooling assembly 1211, such as a Stirling cooler or other refrigeration and/or cooling assembly constructed and arranged to maintain one or more components of sensor assembly 1210 such as optical components at a predetermined temperature, for example, a cryogenic temperature of 77K, such as to improve the sensitivity, accuracy, noise characteristics or response time of sensor assembly 1210.

Sensor assembly 1210 can be positioned on a two-dimensionally (x-y) or three-dimensionally (x-y-z) micro-position adjustable table 1212 constructed and arranged to allow precise positioning of the proximal end of fiber 210 of fiber assembly 1140 relative to one or more optical components of sensor assembly 500 such as a proximal optics 1215, for example, a focusing lens shown in FIG. 12. The system also has the ability to adjust for pitch, yaw, and/or roll for precise positioning. Precise positioning can occur in this manner by manual or automatic calibration, Such calibration can occur at the time of manufacture, when the unit is installed at a medical facility, or, optionally, in the field, at the time of a procedure. The optical components of sensor assembly 1210 can be constructed and arranged to focus IR energy or the like received from the proximal end of the fiber 210 onto an IR detector (not shown) at the sensor assembly 1210.

Sensor assembly 1210 includes a housing 1213 that partially or fully surrounds optical components such as an IR detector (not shown) or the like that can receive electromagnetic energy such as electromagnetic energy at IR wavelengths, from a fiber (see, for example, FIG. 12) of fiber assembly 1140. One or more ports 1214 can extend from sensor housing 1213, for permitting wires or the like coupled to electronic components within the sensor housing 1213 or other regions of the sensor assembly 1210 to communicate with other components of patient interface unit 1120. Proximal optics 1215 can be positioned in a housing 1217 that is coupled to rotary motor assembly 1220. Proximal optics 1215 can be configured to focus collected IR energy or related energy received from fiber 210, and focus the IR energy to a detector or the like (not shown) at sensor assembly 1210. The proximal end surface of fiber 210 (see FIG. 12) can be separated from proximal optics 1215 by a predetermined distance, which can be adjusted, calibrated, or the like by adjustable table 1212. In some embodiments, this adjustment is performed during manufacture. In some embodiments this adjustment is performed in the field, either manually or automatically, preferably adjusted mechanically in manufacturing for optimizing signals generated from the system. As described herein, in some embodiments, rotary motor assembly 1220 can be positioned on the linear translation motor assembly 1230, whereby the combination can rotate and/or translate fiber assembly 1140 relative to outer sheath 1136 of probe assembly 1130. More specifically, rotary motor assembly 1220 can be coupled to a mount 1226, which in turn is fixedly coupled to translation table 1229, for example, by bolts 1231 (see FIG. 12), and/or other fastening devices, adhesives, and so on. In some embodiments, rotary motor assembly 1220 includes a rotary motor 1222 at least partially surrounded by a housing 1221, which is fixedly attached to translation table 1229 and maintains a relative position of rotary motor 1222. In some embodiments, rotary motor assembly 1220 includes a central hollow shaft 1223 into which a probe connector 1260 holding a proximal end 1141 of fiber assembly 1140 can be positioned. Motor 1222 can include a stator, rotor, and/or other well-known rotary motor components, which in turn can initiate a rotary motion in hollow shaft 1223, and therefore, the probe connector 1260 positioned in shaft 1223.

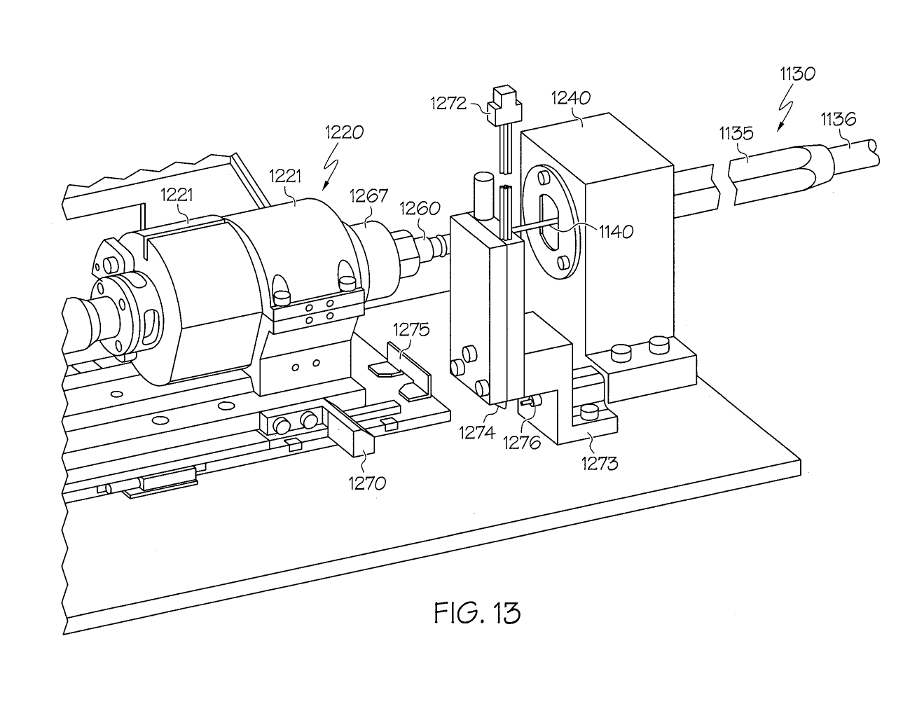

In some embodiments, as shown in FIGS. 11 and 13, patient interface unit 1120 can further include a locking arm 1270 coupled to translation table 1229 that is constructed and arranged to be removably engaged by an actuator 1273 coupled to the base 1250. In the present embodiment, the locking mechanism 1270 and corresponding actuator 1273 are positioned at a region proximal the sheath attachment frame 1240; however, other locations are applicable. In some embodiments, the actuator 1273 includes a reciprocating plunger or pin 1274 shown in FIG. 13. In some embodiments, the plunger 1274 is spring-biased to be in an extended position. The plunger 1274 may include an angled surface that interfaces with an angled surface of the locking arm so they slidably engage each other to capture each other in a clocked position at opposed flat faces. When locking arm 1270 is engaged by the extended plunger 1274 of actuator 1273, the translation table 1229 is positioned against a back region of the locking mechanism 1270 to prevent the locking arm 1270, and therefore the translation table 1229, from moving in the linear direction. This permits components on the translation table 1229 such as the rotary motor assembly 1220 to be placed in a stationary state, for example when patient interface unit 1120 is not in operation, for example, during transportation of the patient interface unit 1120, or between procedures when the unit is operational and in the field. In some embodiments, the translation table 1229 is in the stationary state, and locked in place by the actuator 1273 at a time when the sensor assembly 1230 is to be engaged or disengaged from the patient interface unit 1120. In some embodiments, the plunger 1274 can be retracted by a solenoid controlled by applying a specified voltage to wires 1272. Hard stop 1275 can be included to prevent the translation table 1229 from over-translating or from otherwise traveling. In some embodiments, the hard stop can interfere with a feature on the attachment frame 1240 to prevent over-translation. Plunger 1276 can be spring-biased to limit or prevent vibration or rattling of the translation table 1229 when the plunger 1274 holds the table in a locked position. The plunger 1276 can be equipped with a sensor or switch to confirm the position of the table 1229.

FIG. 12 is a close-up perspective cross-sectional view of an interface region between detector 1213 and the rotary motor assembly 1220 of patient interface unit of FIGS. 9-11, consistent with some embodiments of the present inventive concepts.

As shown in FIG. 12, the rotary motor assembly 1220 includes a cylindrical stator 1222 and cylindrical rotor shaft 1223 through a central portion of the stator 1222. The rotor shaft 1223 rotatably engages the stator 1222 at bearings 1224. A rotational encoder wheel 1228 is fixedly attached to an end of rotor shaft 1223, which can be tapered, conical, circular, or other shape that provides benefits described herein. The encoder wheel provides feedback to the motor controller to precisely control the angular position, angular velocity, or angular acceleration of the rotor shaft 1223 relative to the stator. In this manner, the rotation of the inserted probe connector 1260 and, in turn, rotation of the corresponding fiber 1140, can be precisely controlled.

The end of shaft 1223 can be concave and conical or otherwise circular for receiving a mating convex nose 1264 of the probe assembly 1130, The conical or circular arrangement allows for reliable optical coupling between the proximal end of the fiber 1140, at which the collected IR energy signals are output, with the optical element 1215 of the sensor, ensuring proper alignment and spacing therebetween. In alternative embodiments, other concave/convex nose shapes may be employed and are equally applicable to the principles of the inventive concepts. Such shapes can include but not be limited to parabolic, elliptical, semi-spherical, stepped, and the like. In the conical embodiment depicted in FIG. 12, the conical feature ensures capture and seating of the probe in a repeatable, final position where the proximal end of the fiber can maintain concentricity with the sensor optics 1215. Patient interface unit 1120 can be constructed and arranged to include at least one of several different coupling configurations, for receiving and securing probe connector 1260 at patient interface unit 1120, and permitting the fiber assembly 1140 to translate and/or rotate independently of the sheath, handle, probe connector and/or other elements of the probe assembly 1130.

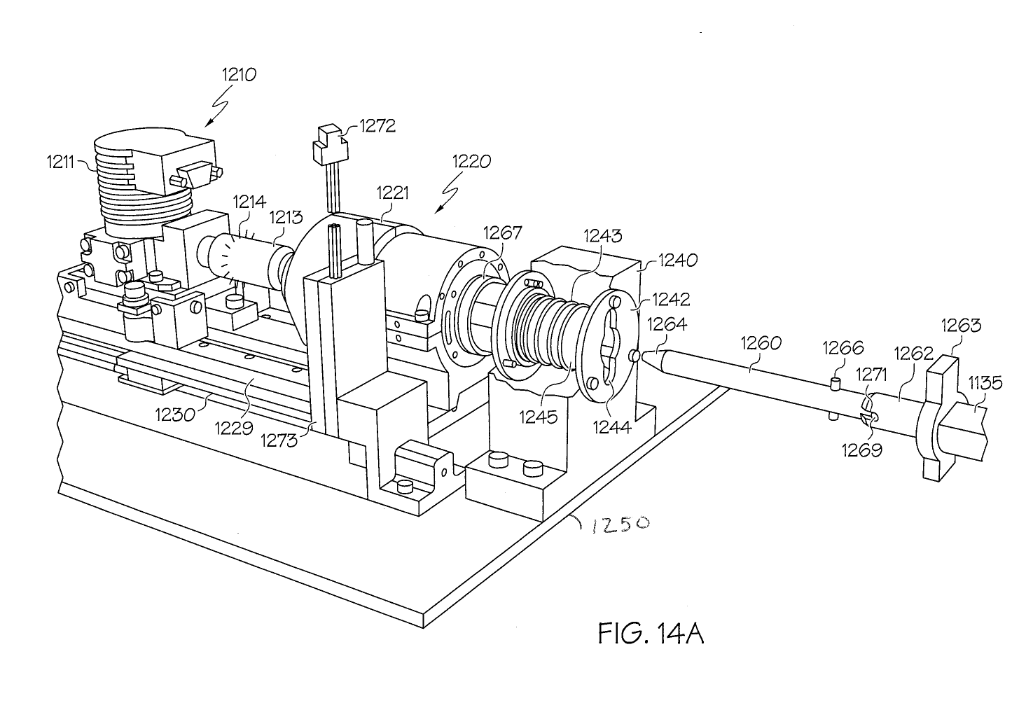

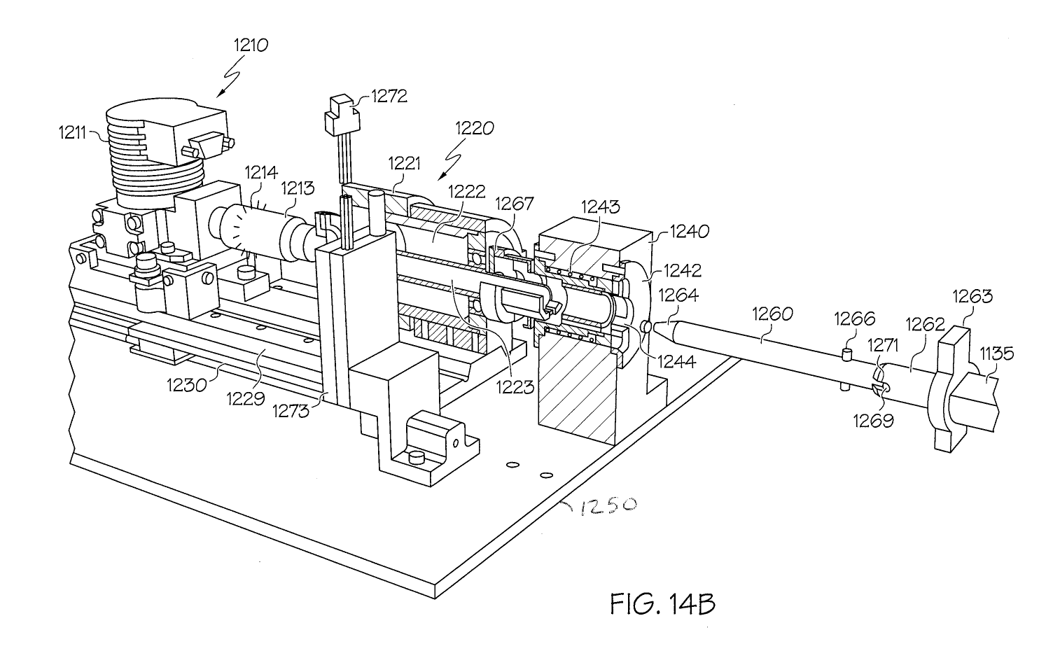

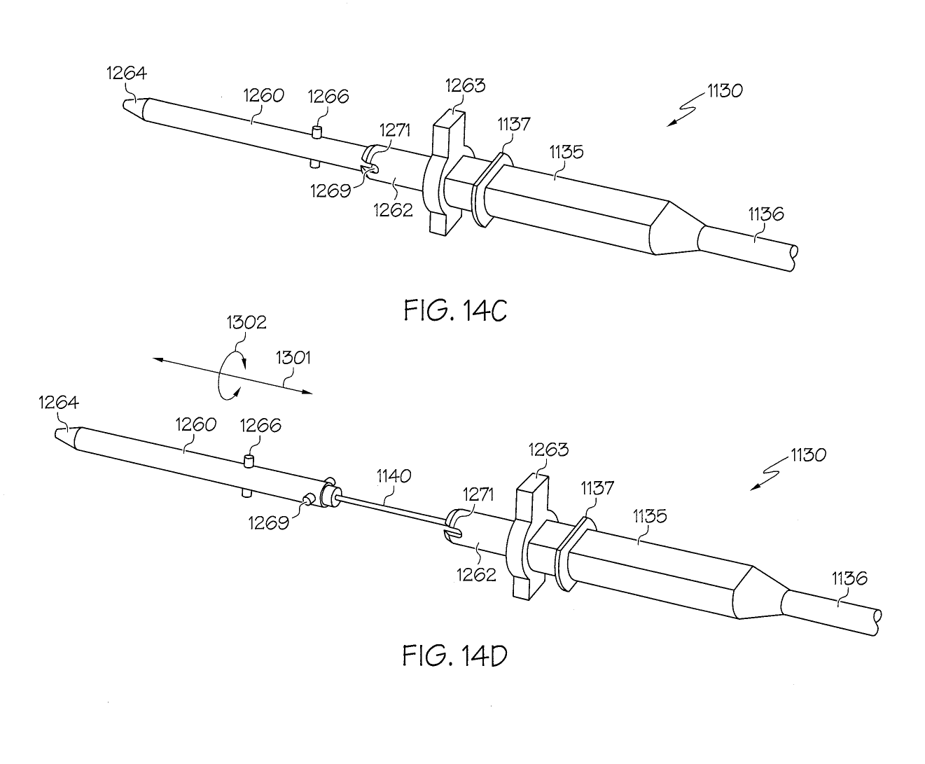

FIG. 14A is a partial cut-away perspective view of patient interface unit 1120 and probe assembly 1130. For example, this view can be considered to illustrate their relative positions during engagement and disengagement of the probe assembly 1130 with the patient interface unit 1120. An interior of a sheath attachment frame unit 1240 coupled to patient interface unit 1120 is likewise illustrated, consistent with some embodiments of the present inventive concepts. In some embodiments, the handle 1135 of the probe assembly 1130 is configured to be mounted to the sheath attachment frame 1240. FIG. 14B is a perspective partial cross-sectional view of elements of patient interface unit 1120 of FIG. 14A, consistent with some embodiments of the present inventive concepts. FIGS. 14C and 14D are perspective views of the interaction of the handle and the probe connector of the probe assembly, in accordance with some embodiments of the present inventive concepts.

Sheath attachment frame 1240 is fixedly mounted to base 1250 and can include an input element 1242 with a slot 1244 for receiving the handle 1135 of probe 1130. More specifically, in some embodiments, a keyed, bayonet portion 1262 of the handle 1135 can be inserted in the slot 1244 for mounting the handle 1135 to the attachment frame 1240. In the present embodiment, the bayonet portion of the handle 1135 includes a body 1262 and lobes 1263 that mate with corresponding features of the slot 1244 to ensure that handle 1135 can be mounted at a specific orientation. In other embodiments, handle 1135 can be shaped or otherwise configured for other types of couplings, for example, square-shaped for insertion into a corresponding square opening at the sheath attachment frame 1240 to prevent rotation about its longitudinal axis. In other embodiments, pegs, pins, or related male elements can extend from the handle 1135 for interfacing with corresponding female elements at the sheath attachment frame 1240. In other embodiments, male elements can extend from the attachment frame 1240 while the handle 1135 includes female elements. The probe connector 1260 and the handle 1135 are coupled to each other prior to insertion in the patient interface unit 1120, and can be separated from each other during a subsequent operation, for example, as described herein. The bayonet portion 1262, 1263 of the handle 1135 can be constructed and arranged as a twist-lock coupling. In particular, the sheath attachment frame 1240 can include a spring-loaded pin activation element 1243 and corresponding plate 1245 adjacent and aligned with the slot 1244 of the input element 1242 and shaped to receive the lobes 1263 of the handle bayonet 1262. The spring-loaded pin activation element 1243 and plate 1245 element can bias the lobes 1263 and handle 1135 into a secured, seated position when the handle is mounted to the sheath attachment frame 1240. The handle 1135 can further include a flange 1137 that abuts the surface of the input element 1242 during insertion of the handle 1135 to the sheath attachment frame 1240. The flange 1137 may also prevent an operator's hand from interfering with the patient interface unit 1120.

The protruding portion 1263 of the handle 1135 is aligned with the slot 1244 for insertion to the sheath attachment frame 1240. As the bayonet portion of the handle 1135 including the body 1262 and the lobes 1263 is inserted into the slot 1244, the spring-loaded pin activation element 1243 is compressed by the lobes 1263 of the bayonet portion to permit entry into the sheath attachment frame 1240. Here, the handle 1135 can be locked in place at the sheath attachment frame 1240 by rotating the handle 1135 about its longitudinal axis, for example, 90 degrees. In doing so, the lobes 1263 is no longer aligned with the slot 1244 of the input element 1242, and the handle 1135 is locked in place in the sheath attachment frame 1240, for example, behind the input element 1242 so that the force applied by the activation element 1243 in turn holds the lobes 1263 in place against an inner wall of the input element 1242.

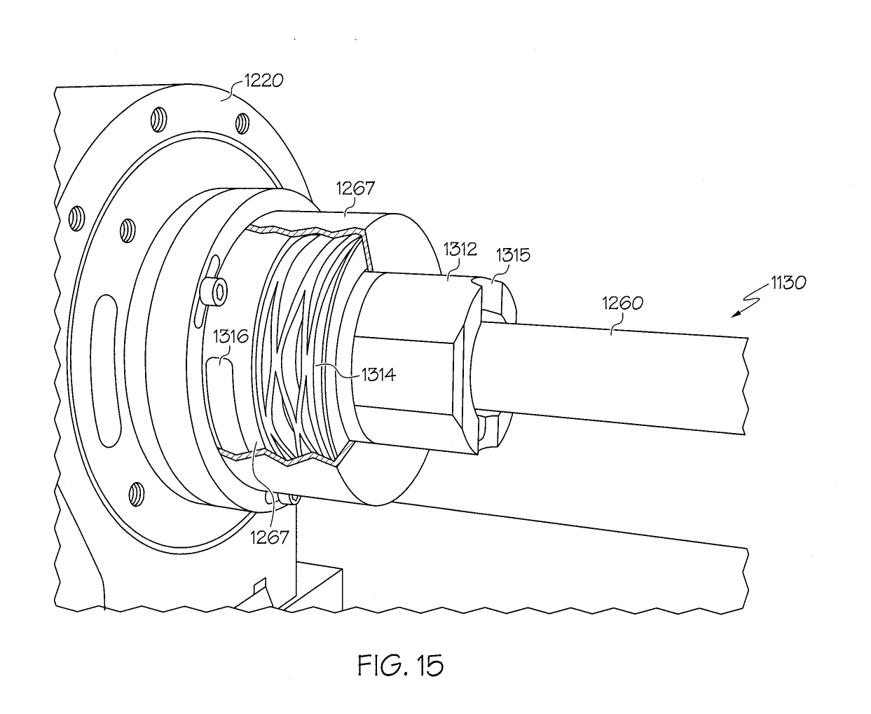

In addition to the handle 1135 being locked in place at the sheath attachment frame 1240, the probe connector 1260 is likewise made to be held in place within the rotary motor 1220. At the same time the handle 1135 is inserted in the sheath attachment frame 1240, the mounting of the handle 1135 contemporaneously operates to mount and fix the probe connector 1260 in the rotary motor 1220. At the time of mounting, the rotary motor 1220 does not translate, as the translation table to which it is mounted is held in place in the linear direction by the locking arm 1270. The rotary motor 1220 is further prevented from rotating during the mounting operation and the angular position of the rotary motor 1220 is controlled so that the pins 1266 on the body of the probe connector are properly aligned with corresponding slots 1313 in an entrance aperture 1312 of the rotary motor 1220. In particular, the nose 1264 of probe connector 1260 extends through sheath attachment frame 1240 and the hollow shaft 1223 (see FIG. 12) to an end of motor assembly 1220, where the outermost end of a fiber is positioned a predetermined distance from an adjacent sensor assembly 1210, or in particular, sensor optics 1215 of sensor assembly 1210. During insertion, probe connector 1260 can be inserted into the entrance aperture 1312 to the rotary motor 1220, as shown in FIG. 15. Probe connector 1260 includes a set of rotary motor engagement pins 1266 that can engage grooves 1213, notches, or other openings in a spring-biased rotary motor coupling 1267 located in housing 1268 during insertion of the probe connector 1260, for example, as shown in FIG. 15. When the handle 1135 is rotated to lock lobes 1263 in the sheath attachment frame 1240, the rotary motor engagement pins 1266 on the body of the probe connector are caused to simultaneously rotate within grooves 1316 of the rotary motor coupling 1267 (see FIG. 15), thereby locking the probe connector 1260 in place at the rotary motor coupling 1267. In the present embodiment, this interaction is accomplished by the interaction of a second set of handle engagement pins 1269 located on the body of the probe connector distal the rotary motor engagement pins 1266. The handle engagement pins 1269 interface with corresponding probe connector slots 1271 of the handle 1135. In this manner, during mounting, a 90 degree turning motion in the handle 1135 results in a corresponding 90 degree turning motion in the probe connector 1260. The same 90 degree turn that operates to couple the handle 1315 to the sheath attachment frame 1240 can simultaneously couple the probe connector 1260 to the rotary motor 1220.

The insertion process is described according to some embodiments as follows:

When the probe 1130 in inserted into patient interface unit 1120, the motor 1220 is stationary in the linear direction. The motor shaft 1223 is free to rotate (lightly detented). The motor shaft pressure cam 1312 is constrained rotationally. In the current embodiment, the constraint is by an engagement between the flats on the motor shaft pressure cam 1312 and the sheath attachment frame 1240. The shaft bayonet engagement pins 1266 engage with slots in the motor shaft 1223 and slots 1315 in the motor shaft pressure cam 1312. Linear translation of the probe stop when the probe proximal ferrule 1264 seats conically in the motor shaft nose 1233. The conical connection maintains axial co-linearity between the fiber 1140 and the proximal optical element 1215. As rotation begins the rotary motor engagement pins 1266 engage with the spring loaded cam surfaces 1316 of the motor shaft pressure cam 1312. Throughout rotation, the cam surface serves to load the spring 1314 which provides pressure at the proximal conical seat interface to hold the probe in place. There exists a detent feature at the end position of the cam surface to prevent accidental decoupling during operation.

After the handle 1135 is locked in place at the sheath attachment frame 1240 and the probe connector 1260 is locked in place at rotary motor 1220, the probe connector 1260 can be separated from the handle 1135 to expose a portion of the fiber assembly 1140, for example, shown at FIGS. 10 and 14D. This separation can occur due to a linear movement of the rotary motor 1220 in a direction away from the sheath attachment frame 1240 during operation, and/or by secondary human interaction through intermediary mechanisms. Referring to FIG. 14D, with the handle 1135 and sheath 1136 fixedly coupled to the handle 1135 and with the probe connector 1260 and its corresponding fiber assembly 1140 separate from the handle 1135, the probe connector 1260 and therefore, its fiber assembly 1140, can undergo linear translation as indicated by arrows 1301 and rotary translation as indicated by arrows 1302 relative to the sheath 1136.

In some embodiments, an optional rigid tube can be inserted that spans the gap between the probe connector 1260 and the handle 1135 to prevent buckling, whipping, etc. during linear translation. Following a procedure, the operational steps can be reversed to disengage the probe assembly 1130, including the handle 1135 and the probe connector 1260, from the patient interface unit 1120.

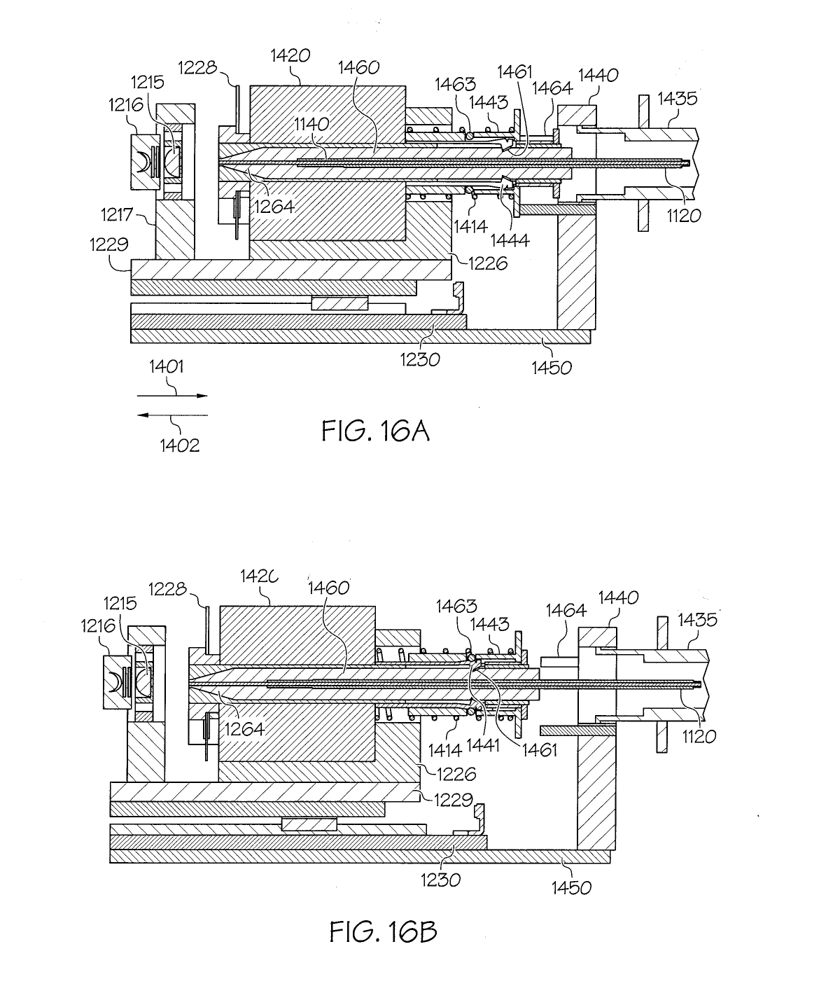

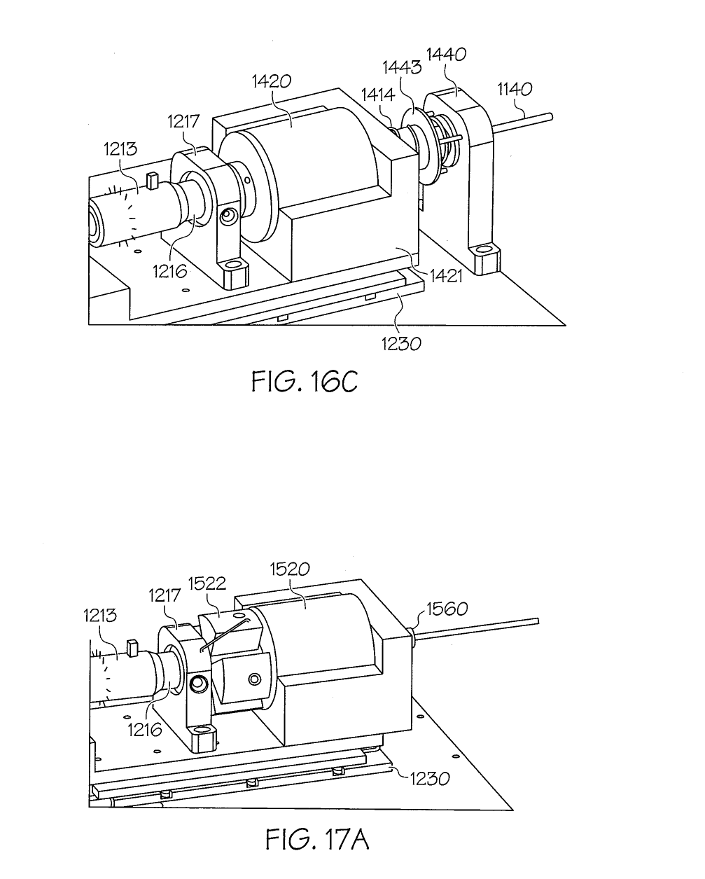

FIG. 16A is a cross-sectional view of a patient interface unit, illustrating a probe connector 1460 during a loading stage, consistent with other embodiments of the present inventive concepts. FIG. 16B is a cross-sectional view of patient interface unit of FIG. 16A, illustrating probe connector 1460 engaged in patient interface unit, consistent with other embodiments of the present inventive concepts. FIG. 16C is a perspective view of patient interface unit of FIGS. 16A and 16B, consistent with other embodiments of the present inventive concepts.

While other embodiments descried herein are directed to manual coupling of the probe connector 1260 to the rotary motor, the present embodiment of FIG. 16A, illustrates automated coupling of the probe connector 1460 to the rotary motor 1420. In the present embodiment, the handle 1435 and the probe connector 1460 of the probe assembly 1130 are inserted through an attachment frame 1440 coupled to a base table 1450 and into a rotary motor 1420 having a hollow shaft, similar to embodiments described herein. Rotary motor 1420 is coupled to a mount 1226, which in turn is coupled to translation table 1229. During a loading stage, as depicted in FIG. 16A, the handle 1435 and its corresponding sheath are mounted to the sheath attachment frame 1440, for example, in a manner similar to FIGS. 14 and 15. Upon detection of an inserted handle, linear motor 1230 drives the translation table 1229 in a direction of arrow 1401 toward the attachment frame 1440 until a connection interface 1443 extending from the rotary motor 1420 abuts a backstop element 1464 extending from attachment frame 1440. The linear motor continues to drive the translation table in the direction of arrow 1401 so that the input element begins to compress spring 1414. A sensor (not shown) can be positioned at or near the backstop element 1464 and/or input element 1442 to detect when the connection interface 1443 is at or near backstop element 1464, and notify the controller of linear motor 1230 to begin to cease movement of the translation table 1229 in the direction of arrow 1401.

Probe connector 1460 can include one or more notches 1461, grooves, openings, capture, helical threads or the like, each constructed and arranged for receiving a finger or other latching element 1444 of a connection interface 1443 extending from a rotary motor housing 1421, which, in the engagement state shown in FIG. 16B can hold probe connector 1460 in place against connection interface 1443. Connection interface 1443 can include a compression spring 1414 for providing resistance with respect to a force applied by the insertion of the probe connector 1460. Thus, when the probe connector 1460 is removed from the patient interface unit by disengaging the latching element 1444 from the latch capture 1461, the spring can provide a force for ejecting the probe connector 1460. The spring 1414 when fully extended can operate to outwardly push the connection interface 1443 toward a distal end of the probe connection. In this manner, a roller 1463 housed in the connection interface 1443 can further operate to bias the latching elements 1444 and securedly seat them in the latch captures 1461.

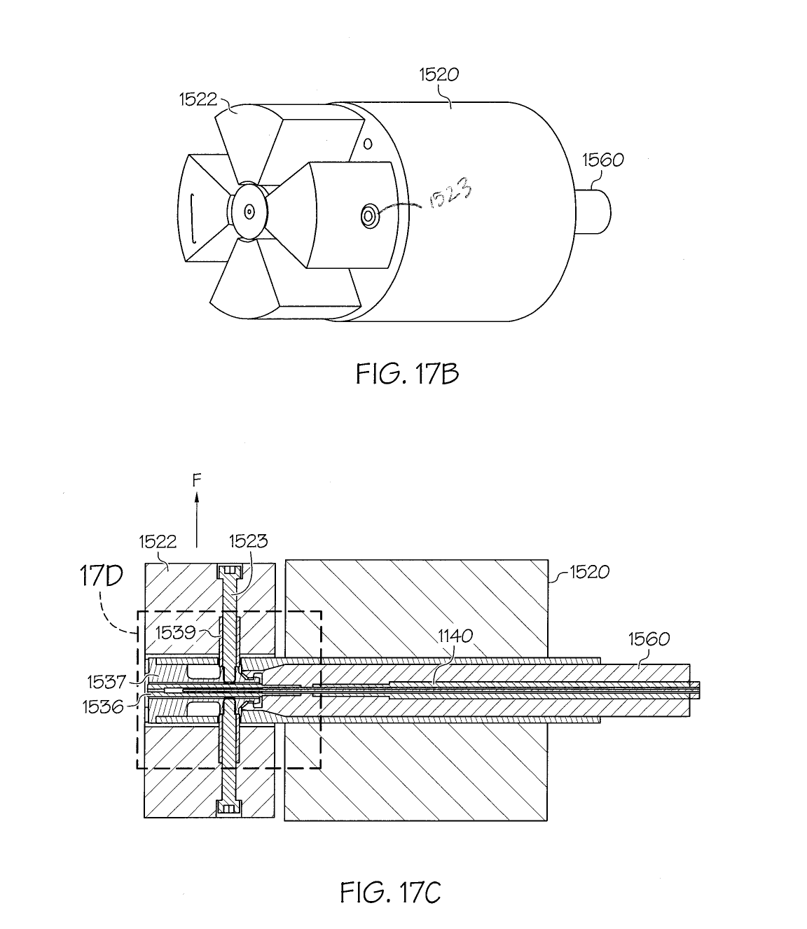

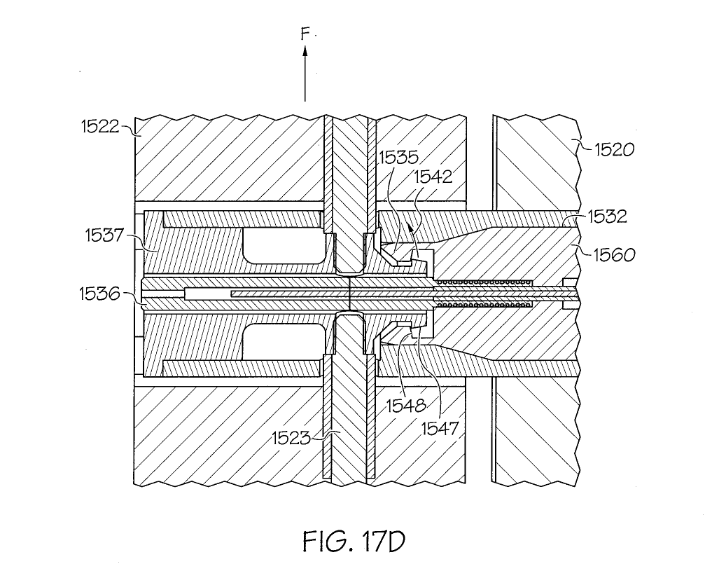

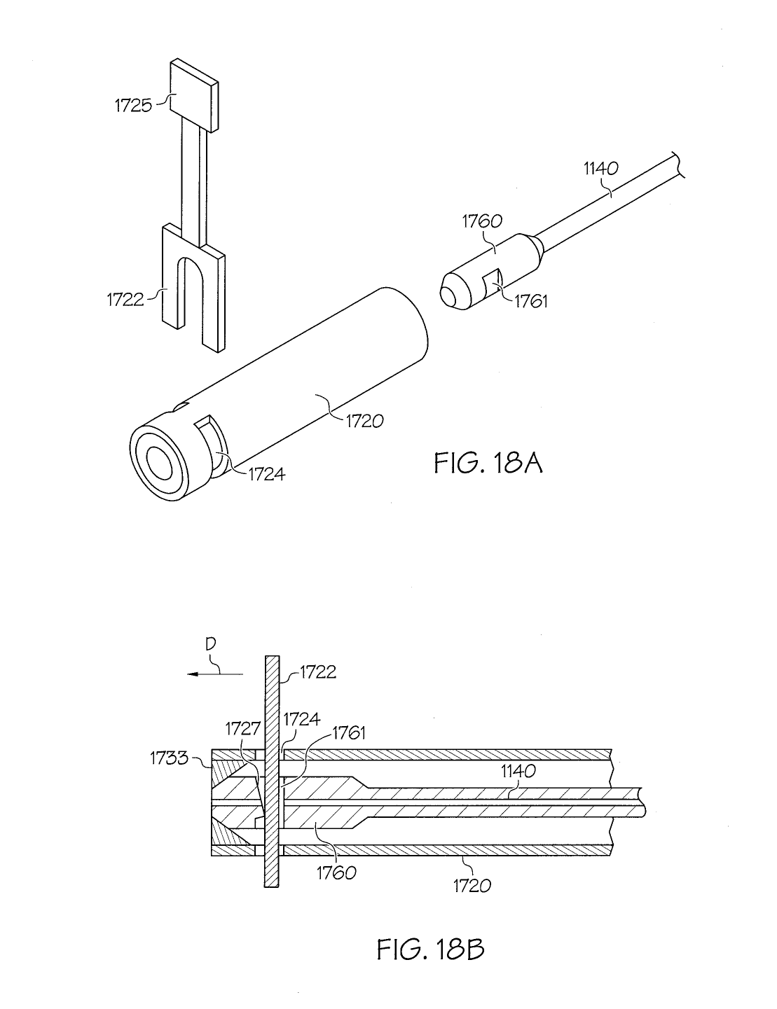

FIG. 17A is a perspective view of a patient interface unit, consistent with other embodiments of the present inventive concepts. FIG. 17B is a close-up perspective view of a rotary motor assembly 1520 of patient interface unit of FIG. 17A, consistent with other embodiments of the present inventive concepts. FIG. 17C is a cross-sectional view of patient interface unit of FIGS. 17A and 18B, consistent with other embodiments of the present inventive concepts. FIG. 17D is a close-up cross-sectional view of patient interface unit of FIGS. 17A-18C, consistent with other embodiments of the present inventive concepts.

In FIGS. 17A-17D, a probe connector 1560 can be inserted in a hollow shaft 1532 of a rotary motor 1520, which rotates the hollow shaft 1532. A plurality of counterweight elements 1522 for providing a centripetal force are coupled to the shaft 1532 at an opposite end of the motor 1520 as an entry point of the motor 1530 for the probe connector 1560. At least one slip bolt 1523 or the like extends through each counterweight element 1522 to apply a force to a removable fiber assembly nose 1536, which can be threaded or otherwise coupled to probe connector 1560. The slip bolt 1523 can be positioned in a slip 1539 providing for movement of the slip bolt 1523 when a centripetal force is applied. A proximal end of a fiber assembly 1140 can be positioned in fiber assembly nose 1536 for positioning at a predetermined distance from a detector, for example, similar to other embodiments herein. Probe assembly 1560 can include a coupling 1535 at a distal end for engaging the fingers of a collet 1537 for locking the probe assembly 1560 in place.

During operation, the shaft 1532, which in turn rotates the counterweight elements 1522 applies a centripetal force F on counterweight elements 1522. This, in turn, pulls slip bolts 1523 in an outward direction, which operates to further engage collet 1537 at surfaces 1547, 1548 in a direction of arrow 1542, thereby locking the probe connector 1560 against collet 1537. Rotational mass can be added to tune stray harmonics that may otherwise occur.

FIG. 18A is a perspective view of a patient interface coupling between a rotary motor assembly shaft 1720 and a probe connector 1760, consistent with other embodiments of the present inventive concepts. FIG. 18B is a cross-sectional view of patient interface coupling of FIG. 18A, consistent with other embodiments of the present inventive concepts.

In the present embodiment, the rotary motor assembly includes an inner shaft 1720 or the like that includes grooves 1724, slots or the like that extend through the wall of the shaft 1720. Two parallel grooves 1724 can be positioned on opposite sides of the shaft 1720. Probe connector 1760 can likewise include grooves 1761, slots or the like that are aligned with the openings 1724 of the shaft 1720.

A linkage device 1722 or the like can be inserted into the rotary motor assembly 1720 so that the ends of the linkage device 1722 are positioned at the shaft openings 1724 and aligned probe connector openings 1761. The linkage device 1722 can be coupled to a control device 1725, for example, a button, switch, actuator, and so on, that controls the insertion and/or removal of the linkage device 1722 with respect to the motor shaft 1720. Accordingly, shaft 1720 and probe connector 1760 are coupled to each other while linkage device 1722 is positioned in the aligned grooves 1761 and openings 1724, respectively, whereby probe connector 1760 is prevented from moving axially with respect shaft 1720, and whereby probe connector 1760 can rotate in response to a corresponding rotation of shaft 1720. The opening 1724 can include a ramp 1727, or taper, angled at the nose 1764 of the probe connector 1760 for applying a force in the axial direction D for abutting a tapered end 1733 of hollow shaft 1720.

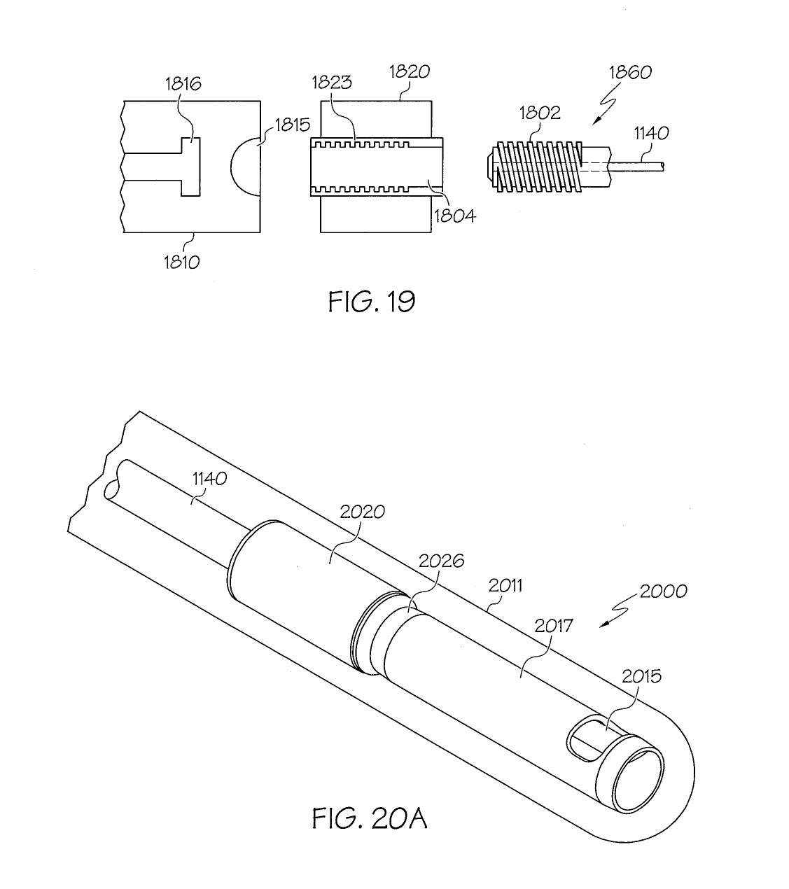

FIG. 19 is a close-up view of a threaded coupling between a probe connector 1860 and a rotary motor 1820 of a patient interface unit, consistent with some embodiments of the present inventive concepts.

Probe coupling 1860 includes a threaded tip 1802 that can mate with a threaded opening 1804 in rotary motor 1820, for example, in a hollow shaft 1823 rotatably positioned at the rotary motor 1820. Probe tip 1802 can be chamfered for alignment. Fiber assembly 1140 can be positioned in probe coupling 1860 in a manner that permits a fiber (not shown) in fiber assembly 1140 to be aligned with respect to a proximal optics 1815, such as a focusing lens positioned between a sensor assembly 1810 and rotary motor 1820.

During insertion of probe coupling 1860, rotary motor 1820 is preferably parked, or locked, so that shaft 1823 does not rotate. Probe coupling 1860 can include a handle (not shown) at its distal end, for example, similar to FIGS. 14C and 14D. The handle can be inserted into, and coupled to a sheath attachment frame, base, or the like in a manner similar to other embodiments described herein.

A translation table or the like, for example, similar as translation table 1229 above, can be driven by a linear motor to move the rotary motor 1820 towards the handle, for example, in response to a determination that the handle is inserted in the sheath attachment frame. A sensor (not shown) can be provided at the sheath attachment frame that detects when the handle is inserted at the sheath attachment frame, which may trigger a linear motion of the translation table. As the motor 1820 moves towards the handle, motor shaft 1823 can rotate in a first direction, for example, clockwise, such that stationary probe 1860 is inserted into, and threaded at, the shaft 1823. The probe tip threads 1802 can be arranged for proper orientation with respect to an operational rotation of motor, so that the probe 1860 does not become dislodged during operation, for example, an operation where rotary motor 1820 rotates fiber assembly 1140 during IR energy collection. The pitch of the threads, rotational speed of the spin motor, and linear speed of the translation table can be coordinated such that the male and female threads mesh as the components converge.

Once probe 1860 is seated (threaded) in the opening 1804 of rotary motor shaft 1823, motor 1820 can cease to rotate, for example, automatically. The translation table can slide linearly in a direction away from the handle to disengage threaded probe 1860 from handle. Probe 1860 can rotate in the first (insertion) direction during an operation, for example, during IR energy collection, thus ensuring that the two components remain engaged.

At the end of an IR collection procedure or other operation involving probe 1260, probe 1260 can be re-engaged with the handle by translating the translation table toward the handle. Probe 1860 can be disengaged, for example, removed from the assembly, by rotating motor shaft 1823 in the second direction, for example, counterclockwise, while the linear motor translates the translation table in a direction away from the handle.

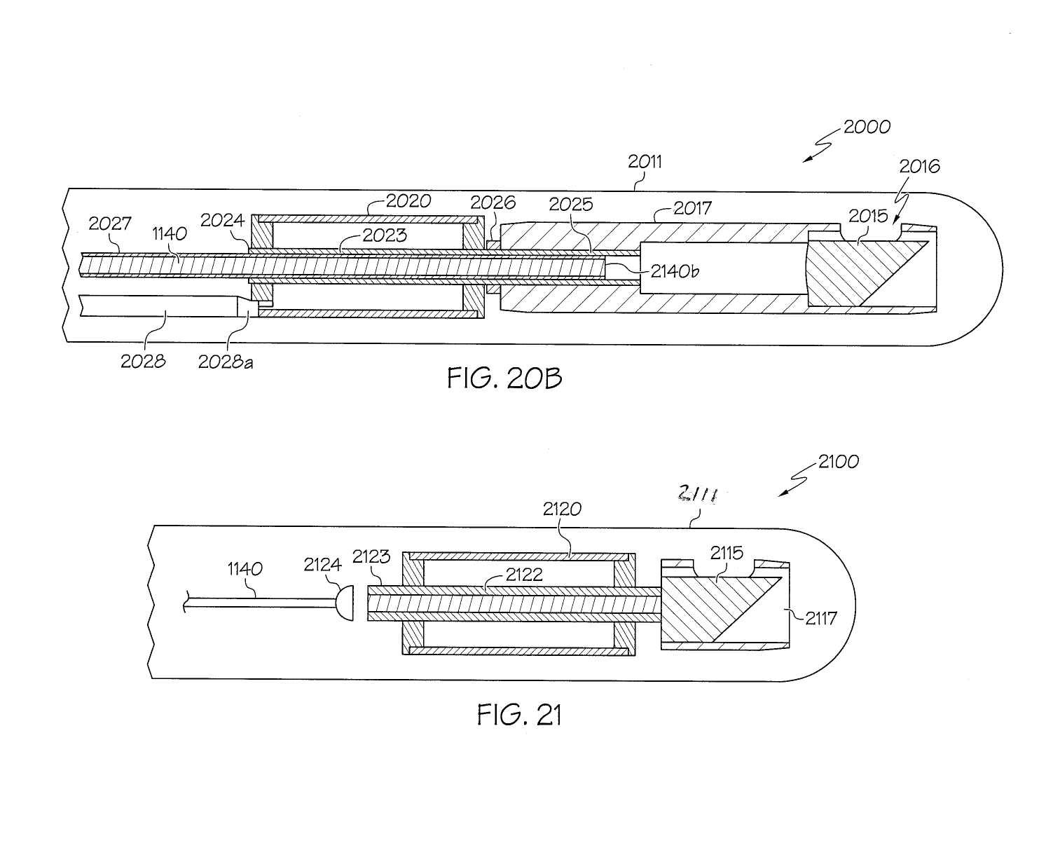

FIG. 20A is a perspective view of a distal end of a probe 2000, consistent with some embodiments of the present inventive concepts. FIG. 20B is a cross-sectional view of the distal end of probe 2000 of FIG. 20A, consistent with some embodiments of the present inventive concepts.

In the present embodiment, the probe 2000 includes a rotary motor 2020 and an optical element 2015 positioned at a distal end of the probe sheath 2011. In this embodiment, a mounting sleeve 2017 is coupled to a rotatable hollow shaft 2023 of motor 2020 at a distal end thereof. A separating element 2026 can be positioned about an exposed region of motor shaft 2023 between rotary motor 2020 and mounting sleeve 2017. In various embodiments, the separating element can comprise a lubricious material, a bearing, or a running gap. Rotary motor 2020 is configured for positioning within probe sheath 2011, which in turn can be inserted in a body lumen. For example, in some embodiments, rotary motor 2020 can have an outer diameter of about 2.4 mm, or less. The probe 2000 can include an electrical connector 2028a for coupling conductive wires 2028 or the like to rotary motor 2020, for example, to supply power to rotary motor 2020. Probe sheath 2011 can be formed of materials that provide at least some amount of transparency with respect to the input and output of IR or related electromagnetic energy to/from probe 2000. At least a portion of the probe sheath 2011, for example, a distal tip of probe sheath 2011, can include an infrared-opaque region.

Shaft 2023 has a proximal end 2024 and a distal end 2025. Optical element mounting sleeve 2017 can be coupled to the distal end 2025 of shaft 2023 to rotate with the shaft 2023. Optical element 2015 can include a mirror, prism, index-matched epoxy, or the like, or a combination thereof. In other embodiments, an optical element is positioned at the distal end 2025 of hollow shaft 2023 proximal a distal end of fiber assembly 1140 extending through hollow shaft 2023. Optical element 2015 is positioned at an opening in mounting sleeve 2017 for receiving IR energy or related electromagnetic signal, for example, from organic tissue at or near a surface of probe sheath 2011. Optical element 2015 is configured to direct the received IR energy to a distal end 2140b of fiber assembly 1140, which in turn outputs the IR energy or the like to a sensor in communication with the proximal end of the fiber assembly 1140, similar to other embodiments herein. Accordingly, the optical element can be optically coupled to the distal end 2140b of the fiber assembly so as to minimize optical loss therebetween.

A slip ring 2027, bearing, lubricious sleeve or the like can be positioned between fiber 1140 and hollow shaft 2023 so that motor 2020 can rotate shaft 2023, and therefore optical element 2015, in an unrestrained and continuous or intermittent manner about the stationary fiber assembly 1140 surrounded by slip ring 2027. Slip ring 2027 coupled with an exposed region of the shaft 2023 at a proximal end of the motor 2020 may assist with the alignment of a stationary lens (not shown) adjacent a proximal side of fiber assembly 1140. In the present embodiment, the slip ring 2027 remains stationary as the motor shaft 2023 revolves around it. The slip ring facilitates this action, as it operates as a bearing between the motor shaft 2023 and the fiber assembly 1140. The tolerances of the fiber assembly 1140, slip ring 2027, and motor shaft 2023 can be chosen such that the fiber assembly 1140 remains centered in the motor shaft 2023 during all states of motor rotation. The fiber assembly 1140 can be maintained to be co-axial with the optical element 2015 by the geometry and position of the hollow motor shaft 2023. In the embodiment of FIG. 20A, 20B, rotational translation of the optical element 2015 is effected by the rotary motor 2020 at a distal end of the fiber assembly 1140, while linear translation of the optical element relative to the sheath 2011 can be effected by a linear motor positioned at a proximal end of the fiber assembly 1140, for example, as described in connection with the various embodiments described herein.

FIG. 21 is a cross-sectional view of the distal end of a probe 2100, consistent with some embodiments of the present inventive concepts.

In this embodiment, probe 2100 includes a motor 2120 and an optical element 2115 in a mounting sleeve 2117 coupled to a rotatable hollow shaft 2123 of rotary motor 2120 at a distal end thereof, consistent with some embodiments of the present inventive concepts, which can be positioned in a probe sheath 2111.

Optical element mounting sleeve 2117 can be coupled to a distal end of the shaft 2123 to rotate with the shaft 2123. Optical element 2115 can include a mirror, prism, index-matched epoxy, or the like, or a combination thereof. An index-matched optical element 2122, for example, formed of an index-matched epoxy or related material, can be positioned in the shaft 2123, and extend along a length of the shaft 2123 between optical element 2115 at the distal end of the shaft 2123 and a stationary focusing lens 2124 or the like at a predetermined distance from the proximal end of the shaft 2123, and positioned between the shaft 2123 and a fiber assembly 1140. In other embodiments, hollow shaft 2123 may house the optical element 2115. In other embodiments, optical element 2115 and index-matched optical element 2122 can be formed of a same material, whereby optical element 2115 includes a lens that has an elongate configuration for positioning in the shaft 2123. In the present embodiment, the system can communicate with a linear translation assembly at a proximal end of the probe 2100, for example as described herein, for providing linear translation of the distal motor 2120. In some embodiments, the present configuration, whereby the rotary motor is positioned at a distal portion of the probe, system signal-to-noise ratio can be improved and reliability and system lifespan can be increased.

During operation, optical element 2115 can receive IR energy from tissue at or near a surface of the probe sheath 2111, and direct the IR energy along the index-matched optical element 2122. In doing so, shaft 2123 and distal optical element 2115 in mounting sleeve 2117 can rotate together about a longitudinal axis of probe 2100 while collecting IR energy from a tissue surface. Lens 2124 or the like mounted at the proximal end of motor shaft 2123 can focus the IR energy at fiber assembly 1140, which in turn carries the IR energy to a sensor assembly, for example, similar to or the same as that in other embodiments described herein.

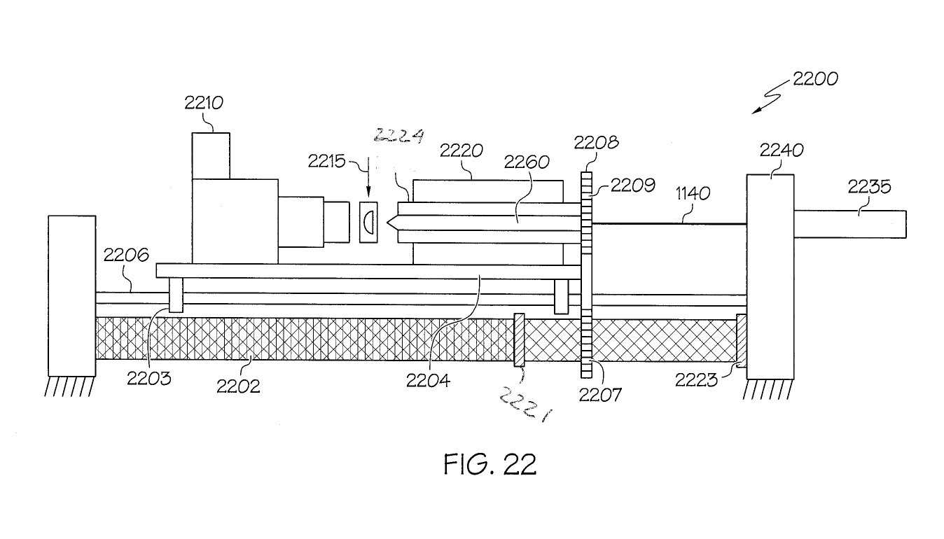

FIG. 22 is a view of a patient interface unit 2200, consistent with some embodiments of the present inventive concepts. Patient interface unit 2200 includes a stationary drive screw 2202, for example, a Yankee screw. As described herein, a Yankee screw 2202 can include a clockwise and counterclockwise helix thread or groove which merge at either end. A gear assembly 2208 communicates with stationary drive screw 2202, and includes a set of gears 2207 that engage with the screw 2202 for moving coupling 2208 in a linear direction back and forth along a length of screw 2202, for example, with respect to an attachment frame sheath 2240. A first stop 2221 and a second stop 2223 can be positioned along the path of the screw 2202 to provide boundaries or limits with respect to a linear movement of the gear assembly 2208. The stops 2221, 2223 act as turn-around points, forcing the linear slide to change direction.

A rotary motor 2220 is positioned on a translation table base plate 2304. Sensor assembly 2210 and proximal optics 2215, for example, a focusing lens, can also be positioned on base plate 2304 for communicating with fiber assembly 1140 in a similar manner as described in other embodiments herein. One or more linear slide bearings 2203 extend from the base plate 2304 for movably positioning about a linear slide 2206 extending in a linear direction parallel screw 2202. Accordingly, rotary motor 2220 and sensor assembly 2210 can move in a linear direction along the length of linear slide 2206.

Rotary motor 2220 includes a hollow shaft 2204 having one end that engages a second set of gears 2209 of the coupling 2208. The hollow shaft 2224 can be the same as or similar to those of other embodiments herein. Accordingly, probe connector 2260 can be inserted in hollow shaft 2224 and can rotate during operation. When shaft 2224 rotates, first and second gears 2207, 2209 engage each other, which causes the first set of gears 2207 to translate coupling 2208 along screw 2202. In doing so, the motor 2220 translates along linear slide 2206. Fiber assembly 1140 can therefore rotate and/or translate relative to handle 2235 in attachment frame sheath 2240 during an IR energy collection operation.

In some embodiments, linear and/or rotary encoders can be employed to manage position and orientation of the translating elements in a closed-loop arrangement. In the present embodiment, translation speed and rotational speed are related to each other, as they are both driven by the same motor, i.e., rotary motor 2220.

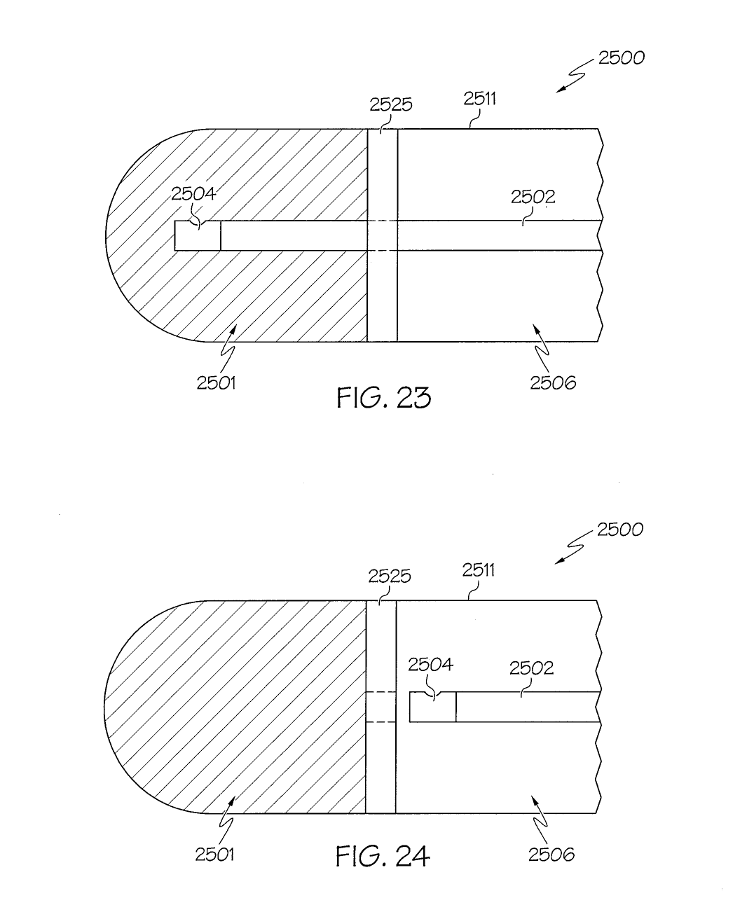

FIG. 23 is a cross-sectional view of a probe 2500 having a distal end of the fiber assembly 2502 located in a first position relative to a distal marker band 2525, consistent with some embodiments of the present inventive concepts. FIG. 24 is a cross-sectional view of probe 2500 of FIG. 23, wherein the distal end of fiber assembly 2502 is in a second position relative to the distal marker band 2525, consistent with some embodiments of the present inventive concepts.

In the present embodiments, probe 2500 includes a probe sheath 2511 through which fiber assembly 2502 is movably positioned. Marker band 2525 can be positioned about a distal end of probe sheath 2511. Probe sheath 2511 can include an IR opaque region 2501, specifically, positioned from the distal marker band 2525 to an outermost distal end of the probe sheath 2501. IR opaque region 2501 may include a coating, shrink wrap, layer of radiopaque material such as those materials cited herein with respect to marker bands 125 referred to above, or the like, at the distal end of the probe sheath 2511. In some embodiments, the IR opaque region is fully or partially opaque to electromagnetic energy at IR wavelengths. In some embodiments, IR opaque region 2501 is constructed and arranged for establishing a location of a distal end of the fiber assembly 2502, for example, establishing whether the distal end of the fiber assembly is positioned in the IR opaque region 2501, or in an IR collection region 2506 of the sheath 2511 at which IR data can be collected from a source external to the probe sheath 2511.

Distal marker band 2525 can be structurally and/or functionally similar to a band referred to in other embodiments herein, for example, bands 125 described in FIG. 2. For example, distal band 2525 can comprise a material of a known emissivity selected from the group consisting of: a thermally conductive material; aluminum, titanium, gold, copper, steel; and combinations of these. Thus, marker band 2525 can be constructed and arranged such that when an IR energy collector, e.g., optical element 2504 at distal end of fiber assembly 2502, is positioned at marker band 2525 (e.g. collects IR energy from marker band 2525), sensor assembly 1210 receives the collected IR energy corresponding to a pre-determined pattern of infrared reflectance or emissivity, or a measurable temperature, which can be compared to temperatures other than the temperature associated with the marker band 2525. In other embodiments, probe sheath 2511 includes an IR opaque region 2501 and an IR transmissive region 2506, with no marker band therebetween. In such embodiments, the transition of the pattern of receipt of IR energy between the IR opaque region 2501 and the IR transmissive region 2506 can serve as an indicator as to optical element 2504 position.

Marker band 2525 can comprise one or more temperature sensors, such as a thermocouple or a thermistor, which can transmit temperature sensor information to a sensor assembly and/or processor. In these embodiments, a temperature reading received from band 2525 can be correlated to the IR energy collected at that location by a fiber assembly collector, such as to perform a calibration procedure, for example, described herein.

Optical element 2504 coupled to a distal end of fiber assembly 2502 can include one or more optical components selected from the group consisting of: optical fiber; lens; mirror; filter; prism; amplifier; refractor; splitter; polarizer; aperture; and combinations of these. Optical element 2504 can receive IR light or the like from a lumen wall or other tissue source and direct the IR light to at least one fiber in the fiber assembly 2502, which in turn transmits the IR energy to a sensor assembly in communication with the proximal end of the fiber. As described herein, in some embodiments, the optical element 2504 can comprise a distal end of the fiber 2502, for example, a cleaved distal end of an optical fiber of the fiber assembly 2502, or an intermediate portion of the fiber 2502, at which IR energy can be collected and transported down its length.

Marker band 2525 and/or opaque region 2501 can be used for positioning distal end of probe 2500 at a location, for example, at a location within the esophagus most proximate a patient's heart, and that the collection region of fiber assembly 2502 is at an optical viewing region 2506, or IR transmissive region, of the probe sheath 2511.

Accordingly, when a temperature mapping system in accordance with some embodiments is activated for operation, the optical viewing region 2506 can be distinguished from the opaque region 2501 by way of different IR energy collections, and therefore, different temperature readings. This permits a user to determine where the longitudinal position of the distal optical element 2504 is relative to the marker band 2525, and further determine a direction of linear travel of the fiber assembly 2502, for example, in a distal direction (toward the distal end of the probe sheath 2511, or in a proximal direction (away from the distal end of the probe sheath 2511) relative to the proximal edge of the marker band 2525. Marker band 2525 can generate a detectable signal corresponding to IR energy emitted from marker band 2525, distinguished from signals corresponding to IR energy emitted from a tissue surface and/or the opaque region 2501. A processor, for example, processor 1150 of FIG. 9 operating in connection with software loaded in system memory, can process this signal and thereby establish the marker band 2525 as a boundary, whereby a proximal side of the marker band 2525, i.e., at IR transmissive region 2506, can be established as a region for collecting IR data, as distinguished from a distal side of the marker band 2525, i.e., IR opaque region 2501. In doing so, linear translation motor 1230 can reverse directions at a time when the marker band 2525 is detected. For example, the linear translation motor 1230 can translate the fiber assembly 2502 in a first direction toward the distal end of the probe sheath 2511, and change directions when the marker band 2525 is detected, whereby the fiber assembly 2502 is translated in a second linear direction opposite the first direction.

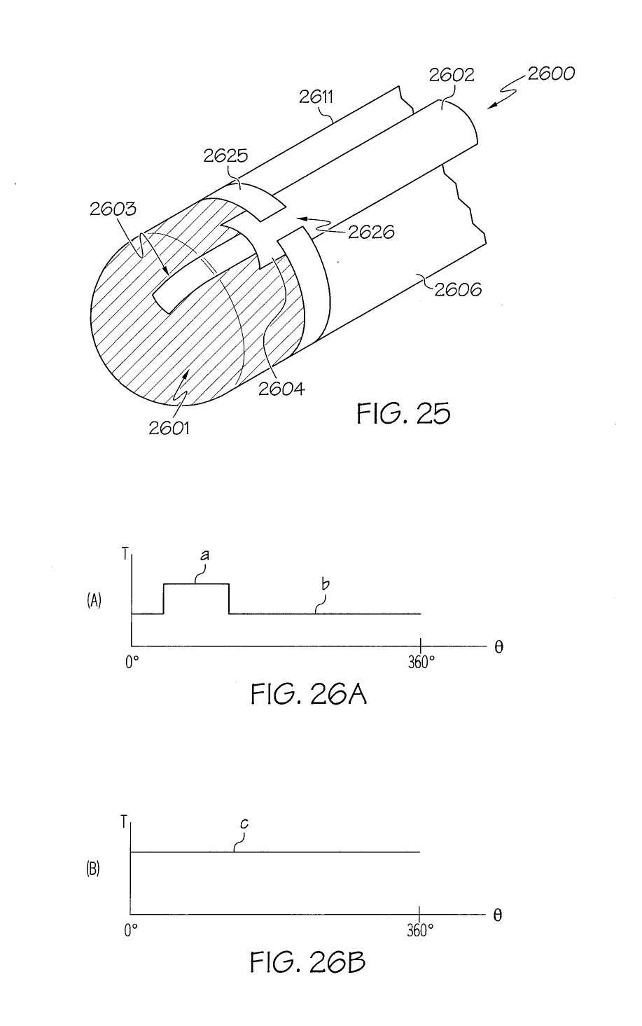

FIG. 25 is a perspective view of a probe 2600 configured to include a C-shaped marker band 2625 about its sheath 2611, which may include an imaging window 2606, consistent with some embodiments of the present inventive concepts. Marker band 2625 is positioned in a similar manner as in other embodiments, for example, circumferentially about at least a portion of, but not the entire 360 degree circumference of, the sheath 2611. At least one gap 2626 can be positioned at the region of the circumference not covered by the marker band 2625. Although a C-shaped geometry having a single gap is shown, other configurations can equally apply, such as one or more marker bands having multiple gaps of varying orientations.

In some embodiments, the gap 2626 can be aligned with an IR transmissive region 2603 that extends in an axial direction along the distal end of the probe 2600, which is not part of the IR opaque region 2601. For example, as shown in FIG. 25, the marker band gap 2626 and the IR transmissive region 2603 can collectively form a continuous path that extends in a longitudinal direction along the distal end of the probe 2600, while the rest of the surface area 2601 of the distal end of the probe 2600 is IR opaque.

The presence of the gap 2626 or corresponding region 2603 permits a reference point to allow for a rotational position of the fiber assembly 2602 to be determined. For example, assuming the distal optics are made to rotate, but not translate, a thermal “A-Scan” similar to that shown in FIG. 26A will result if the distal optics are aligned with the C-Shaped marker band 2625 and/or C-shaped-cross-section IR transmissive region 2603. If this thermal signature is detected, then the system can be triggered to move in a proximal direction back into the target imaging region 2606.

If the thermal “A-Scan” has a signature similar to that of FIG. 26B, then the system will determine that the distal optics 2602 are proximal of the distal marker band 2625, and will need to be moved distally in order to reach the distal-most end of the imaging window 2611.

FIGS. 26A and 26B are graphs illustrating locations, respectively, of a fiber assembly relative to a C-shaped marker band of FIG. 25, consistent with some embodiments of the present inventive concepts.

In FIG. 26A, a collection region at the distal end 2604 of fiber assembly 2602 is at the C-shaped marker band 2625 positioned about probe sheath 2611. Here, the fiber assembly 2602 can rotate 360 degrees about the longitudinal axis along which the probe 2600 extends. Pulse (a) shown at graph A indicates a higher temperature reading at a region of the circumference of the marker band 2625 than the temperature reading (b) at the rest of the circumference of the marker band 2625. Graph 26A therefore indicates that distal end of the fiber assembly 2602 has collected IR data through the IR transmissive gap 2626. Accordingly, marker band 2625 provides a reference point.

Graph 26B includes temperature readings taken at a point along the longitudinal axis of the probe 2600, whereby the distal end 2604 of the fiber assembly 2602 rotates about that point to collect IR energy from a circumference of the probe 2600 relative to the location of the fiber distal end. Here, the temperature readings indicate a higher temperature (c) than the temperature (b) indicating the location radiopaque marker 2625, thereby indicating that the distal end 2604 is at an IR transmissive region, for example, region 2603 of the probe 2600.

Accordingly, in this embodiment, the marker band 2625 has a different emissivity than the tissue visible through the window. Therefore, even in a homogeneous environment, a different signal is received as fiber assembly 2602 moves past the marker band 2625. Differences in emissivity between the marker band 2625 and the tissue can be determined, and from this data, temperature data can be determined.

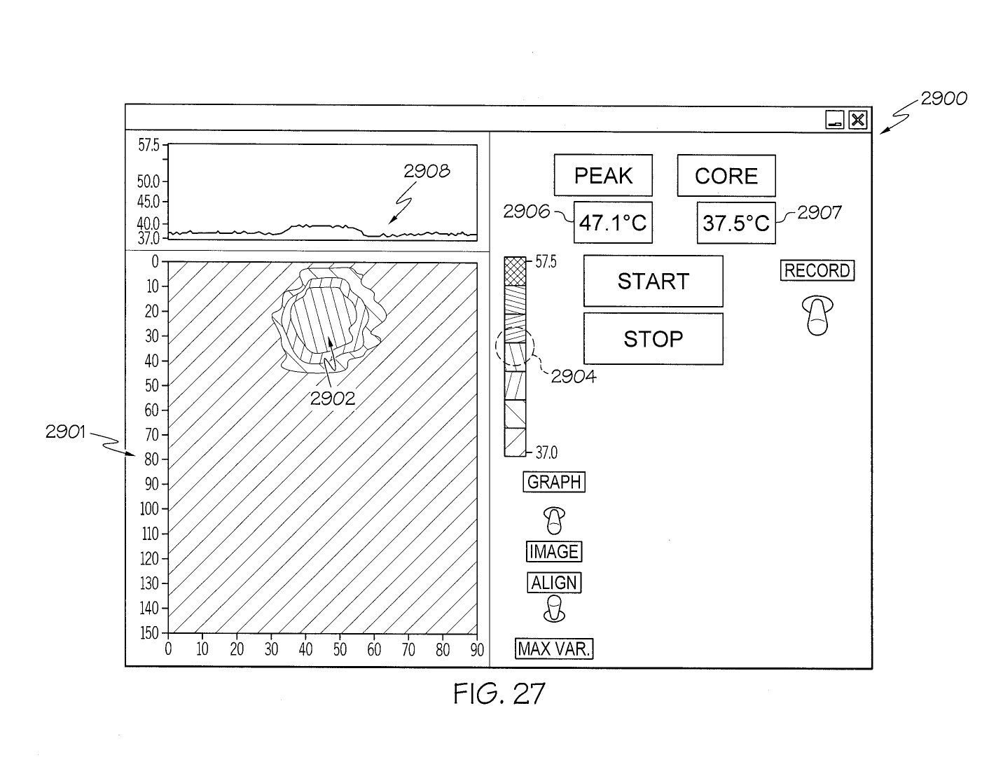

FIG. 27 is an illustration of a display at a monitoring unit 2900, consistent with some embodiments of the present inventive concepts. In describing the display, reference is made to element of a temperature mapping system in accordance with embodiments herein.

As described herein, a probe in accordance with some embodiments positioned in a body lumen such as an esophogus can perform one or more scans of a 360 degree cross-section to collect IR energy received from a tissue surface area of the body lumen. The collected IR energy can correlate to temperatures of the tissue surface areas. A reference temperature, for example, a temperature reading received from a separate tissue temperature measurement means, such as a thermistor or thermocouple, can be correlated to the IR energy collected at that location by the fiber, such as to perform a calibration procedure of system, described herein. Monitor 2900 can display a temperature map correlating to the geometry of the multiple collection locations, for example, as described herein.

For example, a linear assembly described herein can translate a fiber assembly to a spot between two marker bands. The probe can perform a rotational scan of a cross-section of a tissue surface region, referred to as an A-scan. The probe assembly can also perform a scan, referred to as a B-scan, along a length of an IR transmissive region of a probe, for example, at a proximal end of the probe sheath relative to a marker band or opaque region, or between two marker bands. The fiber assembly may also rotate while moving linearly, during which one or more scans may be taken. During the A-scan or the B-scan, multiple IR energy readings may be taken from a surface of a body lumen in which the probe is positioned, permitting a temperature map to be generated.

FIG. 27 illustrates the results of a scan, whereby a processor such as processor 400 or 1150 described herein, can process information signals converted by a sensor, for example, sensor assembly 500 or 1210 herein. Monitoring unit 2900 outputs the results in graphical form, i.e., a temperature map 2901, as shown in the display at monitoring unit 2900. The temperature map 2901 correlates to the geometry of the multiple collection location results of the probe scan. In some embodiments, the multiple collection locations comprise a segment of tubular tissue, such as a segment of esophagus, and temperature map 2901 is a two dimensional representation of the temperature profile of the “unfolded” luminal wall or other body tissue. In other embodiments, a three dimensional representation of temperature profile of the luminal wall or other body tissue can be provided. In other embodiments, a four dimensional representation is provided, for example, including time.

As shown in FIG. 27, a probe scan during an A-scan or a B-scan may reveal a relative “hotspot” 2902, for example, indicating that a region of the body lumen of interest has a temperature that is beyond (above or below) a desired temperature range, or is higher (or lower) than a temperature of other regions of the body lumen, which can be displayed with a display screen temperature map 2901. A corresponding peak temperature, 47.1 degrees C., is shown in display field 2906. A core temperature of 37.5 degrees C. is shown in display field 2907. Core temperature can be determined from an independent thermocouple or thermistor integrated with the probe sheath. Core temperature is measured at a location positioned in or near the body cavity where IR temperature measurement is occurring, but at a distance from the field of interest monitored by the probe. A temperature key 2904 can be displayed with the display screen 2901 for associating the displayed colors of the temperature map to the correct temperature. For example, the hotspot 2902 is estimated according to the temperature key 2904 to be about 47 degrees C. The display field 2906 can provide the corresponding actual peak temperature, 47.1 degrees C., of the hotspot 2902. The display field 2907 can display the core temperature, 37.5 degrees C., of the regions of the temperature map 2901 surrounding the hotspot 2902. A graph 2908 can also be displayed, which depicts the probe A-scan results in a graphical form in addition to or instead of temperature map 2901. In an analogous arrangement, temperature gradients, rates of change in time or space, can be depicted in the display fields as a function of time and in the color-mapping key. As such, the rate of change of temperature and the peak rate of change in temperature, or other parameters can be continuously determined and conveyed to the user. Related inventive concepts involving rates of change of the temperature data consistent with the present inventive concepts are included.

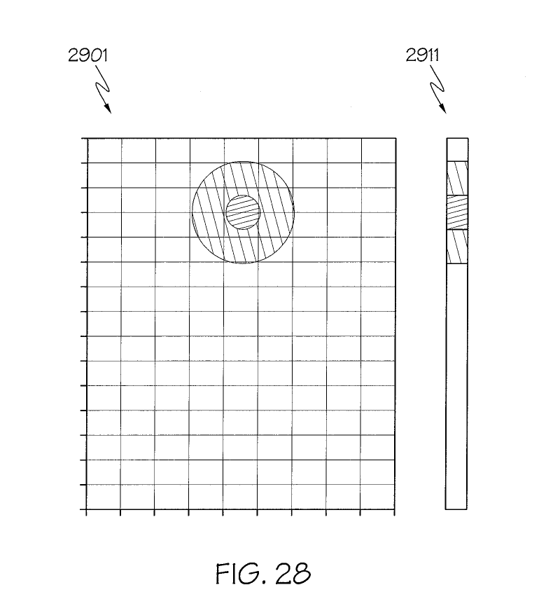

FIG. 28 is an illustration of 2-dimensional (2D) 2901 and 1-dimensional (1D) 2911 temperature map views, respectively, of an IR scan of a tissue surface, consistent with some embodiments of the present inventive concepts. The 2D temperature map 2901 can be similar to or the same as that shown and described with respect to FIG. 27, except that a grid or array is illustrated. The grid or array of elements include a summary statistical representation of some number N neighboring data elements from the full data set. Examples of statistically represented elements include mean, median, and mode of N neighboring elements of temperature or temperature-derived parameters. The present inventive concepts are not limited to the 2D or 1D temperature maps 2901, 2911. Accordingly, other methods for representing temperature data can equally apply. Three-dimensional depictions, where elevation or relative positioning of pixels can be used with or without additional color mapping, can be considered included in this description. Similarly, higher dimensional displays may be considered as additional temperature and physiologic information may be combined onto a single visual representation and updated as a function of time. In addition, temperature at depth from the body lumen surface may be estimated based on the temporal and spatial characteristics of the surface temperature combined with other parameters such as specific heat (J/Kg·K), thermal conductivity (W/m·K), details of the energy source or sources (W/m³), as well as perfusion heat loss (W/m³) and metabolic factors combined with geometric parameters. By fitting the known body lumen surface temperatures, anatomic details and applied energy parameters to predictive models that incorporate the appropriate heat-transfer governing equations, statistically-bound estimates of temperatures within the body beyond the body lumen surface may be obtained and displayed. In FIG. 28, data from an IR scan, for example, performed by a probe in accordance with embodiments herein, can also or alternatively be displayed as a 1D view for easier interpretation and representation to a viewer, such as a physician. An example of when such a display would be warranted is when the axial position information from a B-scan is more important than the specific details of the radial temperature. That is, when the temperature at each anatomic level along the axis of the body lumen is more important than the detailed circumferential temperature information. In a 1D representation 2911, an entire A-scan, or 360-degree (2π) scan may be represented as a single color or shade depicting the peak temperature, average temperature, rate of temperature change, or other temperature parameter, in that scan or some other statistical representation of the A-scan. As with the two-dimensional depiction, M consecutive A-scans may be mathematically or statistically combined to provide temperature or temperature parameter information for a given anatomic level within the body lumen. By way of example, a 5 cm length B-scan could be summarized as a 1D color map 2911 with 10 color segments representing the peak temperature for each 5 mm length of the imaged body lumen. The 2D display 2901 is displayed as an array of rows and columns. The 2D array rows can be converted into singular values using techniques related to a transformation function and/or statistical methods known to those of ordinary skill in the art, such as mean, medium, mode, standard deviation, peak temperature, curve fitting, spatial and temporal transformations, and so on. The 1D display 2911 is displayed as column of data elements. The 1D array rows can be converted into singular values using techniques related to a transformation function and/or statistical methods known to those of ordinary skill in the art, such as mean, medium, mode, standard deviation, peak temperature, curve fitting, spatial and temporal transformations, and so on. Here, a single row or combinations of rows can be transformed into a single representation of temperature at that level, and displayed as the 1D view 2911. These approaches reduce complexity and improve the relevance of thermal mapping data to the viewer.

These techniques can be constructed and arranged to perform such conversions in addition to other techniques for producing other information on the display screen of FIG. 27 and/or FIG. 28, for example, averaging one or more values such as temperature values; finding the peak value of one or more temperature values, comparing peak values of one or more tissue areas, rate of change of tissue temperature, rate of rate of change of tissue temperature; determining an outlier value, determining an area of tissue whose average temperature is higher than other tissue areas measured, or combinations thereof. In some embodiments, alarms or other signals can be generated in response to a determination of one or more of the foregoing.

The foregoing techniques can be performed by a signal processing unit in embodiments herein, for example, processor 400 or 1150 referred to herein.

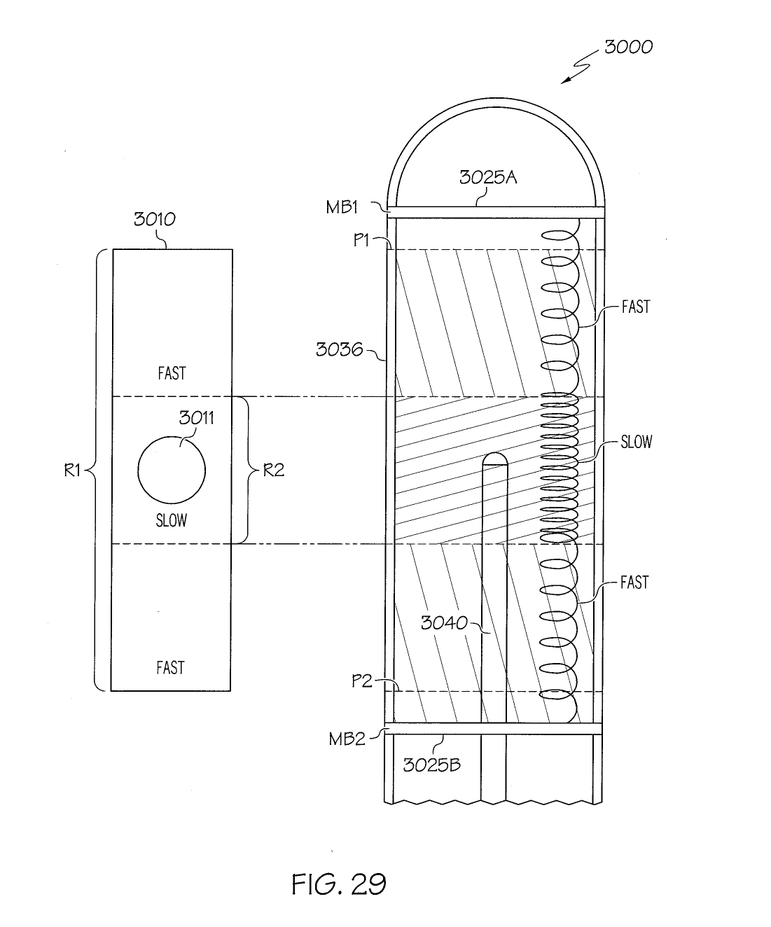

FIG. 29 is a view of a probe 3000 engaged in a multi-mode scanning operation, consistent with some embodiments of the present inventive concepts. Probe 3000 can be the same as or similar to probes of other embodiments herein. Structural details of the probe 3000 are therefore omitted for brevity. In describing the multi-mode scanning operation, reference can be made of elements of temperature measurement system 1100. However, the inventive concepts are not limited thereto.

A linear translation motor assembly, for example, described herein, can control a linear movement of a fiber assembly 3040 relative to a distal marker band 3025a and/or a proximal marker band 3025b about a probe sheath 3036, for example, described herein. A rotary motor assembly, for example, described herein, can control a rotational movement of the fiber assembly 3040.

During a scanning operation, marker band positions MB1 and MB2 can be determined, for example, as described herein. In other embodiments, other longitudinal positions can be determined, for example, positions P1, P2, which are a predetermined longitudinal distance from marker bands 3025A, B, respectively. The region of the probe sheath 3036 between marker band positions MB1 and MB2, or between positions P1 and P2, can be referred to a first region of interest, or R1 as shown in the map 3010 corresponding to a region of a body lumen where probe 3000 is positioned.

Fiber assembly 3040 can collect IR energy or the like from a tissue surface of the body lumen proximal the probe sheath 3036 in the first region of interest R1. Fiber assembly 3040 can be moved linearly and/or rotationally within probe sheath 3036 at a first predetermined velocity, e.g., FAST, by the linear translation motor assembly 1230 and/or rotational motor assembly 1220, respectively, while collecting IR energy or the like. The marker band positions MB1 and MB2 can establish boundary lines, whereby the linear translation motor assembly 1230 changes the direction of movement of the fiber assembly 3040 when positioned at or near a marker band 3035A, B, so that that fiber assembly 3040 performs a scanning operation in the region R1 between the marker bands 3035A, B.

A region of tissue, e.g., hotspot 3011, may be determined to be at a temperature of interest, for example, a temperature that is approaching a critical temperature. A second region of interest R2 can be established that includes the hotspot region 3011 and/or a region surrounding the hotspot region 3011.

The probe 3000 is constructed and arranged to receive and process an amount of temperature data that is dependent on a rate of rotation of fiber assembly 3040. In particular, the faster the rotation at a given translation speed, the more temperature data that can be collected from a tissue surface during a scan while the fiber assembly 3040 translates relative to probe sheath 3036, i.e., a B-Scan. On the other hand, the slower the translation of the fiber assembly 3040 in a longitudinal direction at a given rotational speed, the more temperature data that can also be collected during a B-Scan. Here at a slower translation speed, however, the time required to collect data over a predefined tissue surface region is greater than the time required at a faster translation speed. Accordingly, a slower rotation and a faster translation of the fiber assembly 3040 will permit a greater tissue surface area to be covered but less temperature data will be collected from the surface area.

During operation, an area of interest may be identified. Here, the rotational motor assembly 1220 can increase the rotational speed of the fiber assembly 3040 to thereby increase the resolution and precision of the results with respect to the area of interest. In other embodiments, the rotational speed and translation speed are increased proportionately, thereby increasing the resolution, precision, and frequency of data collection. In doing so, by increasing both the both rotation and translation proportionately, more temperature data can be obtained per A-scan. Accordingly, the output resolution is increased due to the increased number of samples taken per A-scan. Therefore, more data can be received and processed, thereby improving the precision of the resulting measurements. The increase in translation speed can therefore improve the frequency of the measurements over a full B-Scan.

In other embodiments, the translation speed can be reduced while reducing the translation distance to specifically the area of interest, thereby increasing the resolution, precision and frequency of the results at the expense of limiting the scanning area. In other embodiments, the rotational speed can also be increased, which will increase the resolution and precision of the data collection.

Accordingly, to perform the foregoing operation according to a desired resolution, precision, and/or frequency, the linear translation motor assembly 1230 and/or rotational motor assembly 1220 described in embodiments herein can be constructed and arranged to translate the fiber assembly 3040 at two or more different speeds, for example, a first speed FAST when the fiber assembly 3040 is at the first region of interest, and a second speed SLOW when the fiber assembly is at the second region of interest. In other embodiments, the linear translation motor assembly and/or rotational motor assembly can be constructed and arranged to translate and spin the fiber assembly 3040 at more than two speeds, for example, a variable speed whereby the movement of the fiber assembly 3040 is gradually reduced as the temperature of the tissue surface in a region of interest is determined to be at a level that is of higher interest.

As described above, a temperature mapping system can be inserted into an airway, esophagus or other body lumen to monitor ablation progress during a medical procedure such as airway ablation treatment of tumors, chronic obstructive pulmonary disease (COPD), or asthma. The temperature mapping system can also be used to monitor esophageal and/or adjacent airway temperatures for potential off-target damage to nearby tissues during such an ablation procedure. However, the present inventive concepts referred to above are not limited to the foregoing applications, and can apply to other luminal temperature sensing applications.

The generation of IR-derived spatial and temporal thermal patterns according to embodiments herein can be used to estimate on-target therapeutic dose and completeness of energy-based treatments for disorders of epithelial tissues such as Barrett's esophagus, whereby an affected region that is treated using an energy-based ablation application can be monitored to confirm the effectiveness of the treatment by mapping the energy “dose” at each treated location. For example, a temperature map can indicate surface temperatures above or below a specific temperature threshold for a specific amount of time, for determining whether a treatment performed on a tissue region is complete and/or effective. The IR thermal dose map can be overlaid or correlated with images of affected epithelium to ensure that the planned treatment area and the therapeutic dose area are coincident.

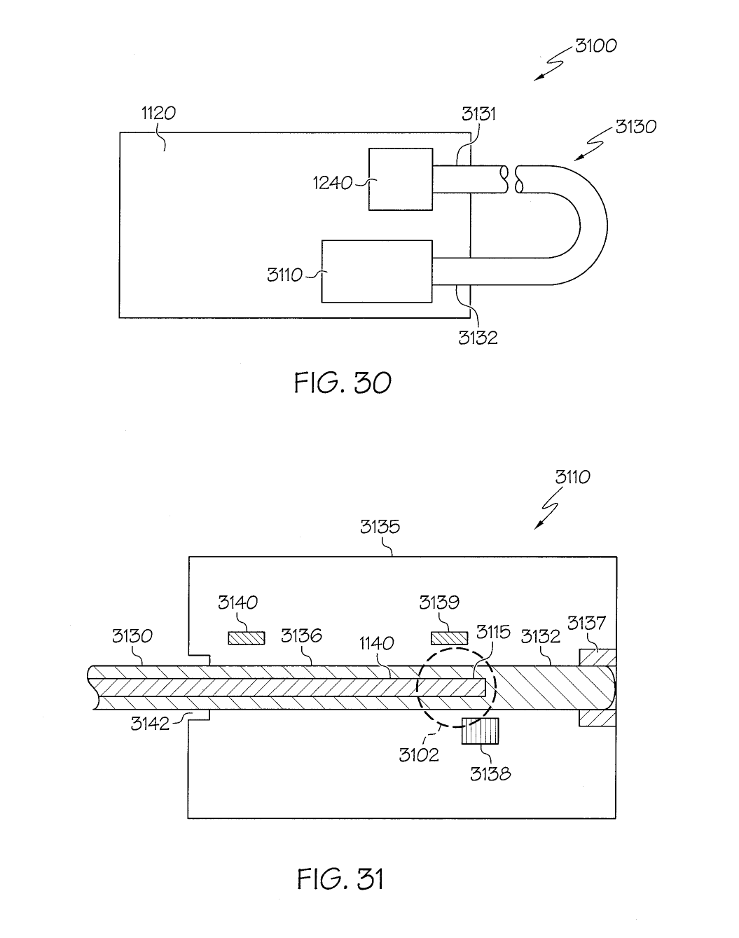

FIG. 30 is a schematic view of a temperature measurement system 3100, consistent with some embodiments of the present inventive concepts.

In some embodiments, the temperature measurement system 3100 can include a patient interface unit 1120, for example, as described herein. In one embodiment, a calibration unit 3110 is positioned at the patient interface unit 1120. In another embodiment, the calibration unit 3110 is separate from the patient interface unit 1120, for example, constructed as a handheld device, and in communication with the patient interface unit 1120 by an electrical connector, wireless communication, or other medium for exchanging signals between other elements of the system 3100 such as the patient interface unit 1120 and/or a processor 1150 (not shown) and the calibration unit 3110. The temperature measurement system 3100 can include a thermal imaging probe assembly 3130 having a first end 3131 removably coupled to a bulkhead unit 1240 or other coupling device at the patient interface unit 1120. A second end 3132 of the probe assembly 3130 can be removably coupled to the calibration unit 3110. The calibration unit 3110 is constructed and arranged to calibrate the system 3100 prior to or at the time of use, for example, at a field location. Calibrating the system 3100 can address and compensate for variability, drift, or the like which may occur during operation, in particular, with respect to the patient interface unit 1120 and/or the probe assembly 3130.

FIG. 31 is a cross-sectional view of the calibration unit 3110 of FIG. 30. The calibration unit 3110 includes a heat source 3138, a first temperature measuring device 3139, and a second temperature measuring device 3140.

The calibration unit 3110 can include a housing 3135 in which the heat source 3138 and thermocouple devices 3139, 3140 are positioned. In some embodiments, the housing 3135 includes an interior that approximates a black body, and is light-tight.

The probe assembly 3130 can be inserted into the calibration unit 3110 at a receiver 3142. In some embodiments, the housing 3135 includes a spherical or cylindrical shaped cavity extending from the receiver 3142 for receiving an inserted probe assembly 3130. The distal end 3132 of the probe assembly 3100 can be held in place in the calibration unit 3110 by a catheter holder 3137, for example, a clamp or related catheter receiving device so that that probe assembly 3130 can be held in place for providing repeatable measurements. A fiber assembly 1140 comprising at least one optical fiber or related infrared signal transport element can be positioned in the probe assembly sheath. The heat source 3138 is positioned in the calibration unit 3110 for heating the interior region the calibration unit 3110 to a temperature. The first temperature measuring device 3139, for example, a thermocouple device is positioned near the heat source 3138 and measures an actual first temperature of the region where a hotspot 3102 is produced by the heat source 3138.

The distal end 3115 of the fiber assembly 1140 can receive IR energy or the like related to the output of the heat source 3138, for example, in a same or similar manner as fibers of other embodiments herein, and output the received IR energy to a detector, for example, in a manner similar or the same as that described in other embodiments herein. The IR data collected by the probe assembly 3130 can be the same as or similar to data described in other embodiments herein, for example, a voltage or current signal that represents a change in received infrared light corresponding to the output of the heat source 3138 received by the fiber assembly 1140.

In other embodiments, temperature sensors (not shown), such as a thermocouple or a thermistor, for example, of the type of temperature sensor 121 of FIG. 2 herein, can be included in probe assembly 1130 for collecting reference temperature data or the like, and can be connected to one or more electrical wires or other information transfer conduits which transmit the temperature sensor information to sensor assembly patient interface unit 1120 and/or monitoring unit 1110.

The second temperature measuring unit 3140, for example, a thermocouple device or the like, is positioned at a separate region of the housing 3135 spaced apart from the region of the hotspot 3102 and the first temperature measuring device 3139, for measuring an actual temperature of the other region.

The system 3100 includes a processor, and associated memory and software, that can be located at the calibration unit 3110 or external to the calibration unit 3110 that determines a relationship between the actual temperatures measured by the first and second temperature measuring devices 3139, 3140, respectively, for example, a difference between the two measured temperatures. Additional temperature measurements can be taken by either or both temperature measuring devices 3139, 3140, or at different temperature measurement devices in order for the system 3100 to perform an accurate calibration.

The processor can also receive and process a voltage variation or the like corresponding to the IR energy output by the IR detector. The temperature and voltage measurements are preferably taken simultaneously or near simultaneously. The processor can compare the measured temperature difference and the voltage variation output by the IR detector. The comparison result can be used to calculate a calibration value in response to the determined relationship and the collected IR data, e.g., voltage variation. The processor can modify subsequent measurements taken by the temperature measurement system 3000, for example, during subsequent use when inserted in a body lumen, in response to the calibration value.

In some embodiments, a probe system performs adaptive scanning using the thermal dynamic properties of tissue, which may provide the ability to filter out noise in the signal by applying calculations to the raw data. For example, during radiofrequency ablation it is not expected to collect IR temperatures below core body temperature as read by an integrated thermocouple. Thus, such readings can be eliminated from a calculation. Spatial information can be incorporated to rule out adjacent temperature readings that would be outside the range of what would be expected in the tissue.

In some embodiments, a system includes an alarm that is activated based of the rate of temperature change over an area of tissue. For example, if the temperature begins to rise rapidly in a given series of scans, then the system could provide an alarm based on the rate of change. In some embodiments, this could be in response to a predictive calculation mechanism that estimates when and how high the temperature may peak, for example, at >0.5° C./sec. With this data the physician can be warned to discontinue an ablation procedure soon enough as to prevent injury to the tissue, or alternatively, the data may be used to automatically discontinue the ablation procedure without requiring further action by the physician.

While embodiments of the devices and methods have been described in reference to the environment in which they were developed, they are merely illustrative of the principles of the inventive concepts. Modification or combinations of the above-described assemblies, other embodiments, configurations, and methods for carrying out the inventive concepts, and variations of aspects of the inventive concepts that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

As will be appreciated by one skilled in the art, aspects of the present inventive concepts may be embodied as a system, method, or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Computer program code for carrying out operations for aspects of the present invention may be written in any combination of one or more programming languages. The program code may execute entirely on the user's computer, partly on the user's computer, as a stand-alone software package, partly on the user's computer and partly on a remote computer or entirely on the remote computer or server.

We claim:

1. A system that produces temperature estimations of a tissue surface, comprising:
a base;
a probe assembly having a proximal end and a distal end, the proximal end of the probe assembly at the base and extending along a longitudinal axis, and including:
a handle at the proximal end of the probe assembly; and
a probe connector;
a fiber assembly extending through the probe assembly, the fiber assembly including at least one fiber constructed and arranged to receive infrared energy from the tissue surface;
a motion unit at the base, the motion unit constructed and arranged to at least one of rotate the at least one fiber relative to the base about the longitudinal axis and translate the at least one fiber relative to the base in a linear direction along the longitudinal axis;
a first coupling mechanism coupled to the base, wherein the handle is removably coupled to the first coupling mechanism; and
a second coupling mechanism at the motion unit, wherein the probe connector is removably coupled to the second coupling mechanism.

2. The system of claim 1, wherein the motion unit comprises:
a rotary motor having a hollow shaft, wherein the probe connector is positioned in the hollow shaft, and wherein the hollow shaft is driven by the motion unit to rotate the at least one fiber about the longitudinal axis; and
a linear motor that translates the at least one fiber and the rotary motor in a linear direction along the longitudinal axis.

3. The system of claim 2, wherein the rotary motor assembly and the linear motor operate independently of each other.

4. The system of claim 1, wherein the motion unit comprises:
a rotary motor having a hollow shaft, wherein the probe connector is positioned in the hollow shaft, and wherein the hollow shaft is driven by the motion unit to rotate the at least one fiber about the longitudinal axis.

5. The system of claim 4, wherein a proximal end of the probe connector includes a conical nose, wherein a proximal end of the at least one fiber is at the conical nose, and wherein a proximal end of the hollow shaft of the rotary motor mates with the conical nose of the probe connector.

6. The system of claim 5, further comprising an optical element adjacent the rotary motor, wherein the conical nose is positioned in the hollow shaft such that the at least one fiber is aligned with the optical element along the longitudinal axis.

7. The system of claim 6, wherein the conical nose of the probe connector is conformably positioned in a conical cavity of the hollow shaft of the rotary motor to maintain concentricity between the at least one fiber and the optical element during operation of the system.

8. The system of claim 4, wherein when the rotary motor rotates between two positions at a predetermined angle between the two positions, the at least one fiber rotates at the same predetermined angle and at the same time as the rotary motor.

9. The system of claim 4, wherein the second coupling mechanism includes a spring-biased rotary motor coupling at the hollow shaft of the rotary motor, the spring-biased rotary motor coupling having at least one groove, and wherein the probe connector includes at least one engagement pin constructed and arranged to mate with the at least one groove at the hollow shaft of the rotary motor.

10. The system of claim 4, further comprising an automatic coupling mechanism that couples the probe connector to the rotary motor by detecting the handle at the first coupling mechanism, and drives a connection interface of the rotary motor to the probe connector for interfacing with the probe connector.

11. The system of claim 4, wherein the rotary motor includes a plurality of counterweights coupled to the hollow shaft for providing a centripetal force, and wherein the second coupling mechanism is positioned at the counterweights for coupling to a proximal end of the probe connector.

12. The system of claim 11, wherein the second coupling mechanism comprises a collet and wherein the probe connector comprises a coupling that interfaces with the collet.

13. The system of claim 4, wherein the probe connector comprises at least one slot, the hollow shaft comprises at least one opening that aligns with the at least one slot of the probe connector, and wherein the system further comprises a linkage device that is positioned in the aligned at least one slot and opening to prevent the probe connector from moving axially with respect to the hollow shaft.

14. The system of claim 13, further comprising a control device that controls an insertion and removal of the linkage device with respect to the hollow shaft.

15. The system of claim 13, wherein the at least one probe connector slot includes a ramp for applying a force in an axial direction for abutting the probe connector with an end of the hollow shaft.

16. The system of claim 4, wherein the hollow shaft of the rotary motor includes a threaded region, and wherein the probe connector comprises a thread that mates with the threaded region of the rotary motor.

17. The system of claim 16, further comprising a sensor at the first coupling mechanism that detects when the handle is coupled at the first coupling mechanism, and wherein the translation table moves the rotary motor in a direction relative to the probe connector for coupling the threaded probe connector with the threaded region of the rotary motor.

18. The system of claim 1, further comprising a linear motor that translates the at least one fiber in a linear direction along the longitudinal axis.

19. The system of claim 18, wherein the motion unit further comprises a translation table that is moved along the base by the linear motor in the linear direction along the longitudinal axis.

20. The system of claim 19, further comprising a locking mechanism coupled to the translation table, and an actuator coupled to the base, wherein the locking mechanism engages the actuator to prevent the translation table from a linear movement.

21. The system of claim 1, wherein the system is constructed and arranged to produce surface temperature estimations of a body cavity having a tissue surface.

22. The system of claim 1, further comprising a sensor assembly having a sensor that receives the infrared energy from the at least one fiber, and converts the received infrared energy into temperature information signals.

23. The system of claim 22, wherein the sensor assembly is positioned on a positioning plate for aligning the sensor assembly with a proximal end of the at least one fiber.

24. The system of claim 23, wherein the positioning plate is a positioning plate for adjusting the sensor assembly in at least one of a pitch, yaw, roll, x, y, and z direction relative to the proximal end of the at least one fiber.

25. The system of claim 22, wherein the sensor assembly comprises a cooling assembly constructed and arranged to cool one or more portions of the sensor.

26. The system of claim 22, further comprising a controller that processes the infrared energy received by the sensor assembly and generates an output that includes temperature data related to the processed infrared energy.

27. The system of claim 1, wherein a portion of the fiber assembly between the probe connector and the first coupling assembly extends in the linear direction along the longitudinal axis during translation of the at least one fiber.

28. The system of claim 27, wherein the at least one fiber extends directly between the first coupling assembly and the motion unit.

29. The system of claim 1, wherein the fiber assembly is passive, and is constructed and arranged to only collect infrared energy from the tissue surface.

30. The system of claim 1, wherein the first coupling mechanism includes a sheath bulkhead coupled to the base and having a slot for receiving the handle of the probe assembly.

31. The system of claim 30, wherein the sheath bulkhead includes a twist lock coupling at the slot, and wherein the handle includes a bayonet portion that mates with the twist lock coupling at the slot to prevent rotation of the handle about the longitudinal axis.

32. The system of claim 31, wherein the twist lock coupling includes a spring-loaded pin activation element and the bayonet portion of the handle includes at least one lobe, and wherein the spring-loaded pin activation element biases the at least one lobe at the sheath bulkhead unit.

33. The system of claim 1, wherein the motion unit comprises a Yankee screw and a rotary motor, wherein the Yankee screw includes a Yankee screw motor that translates the at least one fiber and the rotary motor in a linear direction along the longitudinal axis.

34. The system of claim 33, wherein the Yankee screw motor operates to rotate the Yankee screw, the Yankee screw including dual opposed continuous helical grooves and wherein the Yankee screw motor rotates the Yankee screw to translate the at least one fiber and the rotary motor in the linear direction.

35. The system of claim 33, wherein a translation speed and a rotational speed of the fiber assembly are both driven by the rotary motor.

36. The system of claim 1, wherein the at least one fiber collects infrared energy from a body lumen tissue surface while the rotary motor of the motion unit rotates the at least one fiber about the longitudinal axis.

37. The system of claim 1, wherein the at least one fiber collects infrared energy from a body lumen tissue surface while the motion unit at least one of translates the at least one fiber along the longitudinal axis and rotates the at least one fiber about the longitudinal axis.

38. The system of claim 1, further comprising a controller that processes infrared energy collected by the at least one fiber, and generates an output that includes temperature data related to the processed infrared energy.

39. The system of claim 38, wherein the output includes at least one of a two dimensional (2D) graphical temperature map, a one-dimensional (1D) graphical temperature map, a temperature value, an alarm, and a temperature rate of change.

40. The system of claim 1, wherein the probe assembly further comprises a sheath coupled to the handle, wherein a distal end of the fiber is positioned in the sheath and at least one of translates and rotates relative to the sheath.

41. The system of claim 1, further comprising at least one marker band positioned at a distal end of the sheath, wherein the distal end of the fiber assembly is constructed and arranged to translate relative to the at least one marker band.

42. The system of claim 41, wherein the sheath includes an infrared opaque region at a distal side of the marker band, and an infrared transmissive region at a proximal side of the marker band.

43. The system of claim 41, wherein the at least one marker band comprises a distal band and a proximal band, and wherein the first fiber assembly is constructed and arranged to translate between the distal band and the proximal band.

44. The system of claim 43, wherein the translation assembly is constructed and arranged to translate the fiber in a reciprocating motion between the distal band and the proximal band, and wherein the fiber receives the infrared energy from a region between the distal band and the proximal band.

45. The system of claim 41 wherein the at least one marker band is constructed and arranged to cause a sensor in communication with a proximal end of the at least one fiber to produce a predetermined signal when the distal end of the at least one fiber receives infrared light from the at least one marker band.

46. The system of claim 41 wherein the at least one marker band is C-shaped, and wherein the C-shaped marker band includes two ends, and a gap between the two ends.

47. The system of claim 46, wherein the gap identifies a rotational position of the at least one fiber.

48. The system of claim 46, wherein the gap provides a different and distinguishable signal from the rest of the marker band due to differences in emissivity between tissue and the marker band material.

49. The system of claim 1, further comprising a processor that converts the infrared energy received at the at least one fiber into a plurality of temperature measurements.

50. The system of claim 1, further comprising a display user interface that receives the temperature measurements from the processor, and displays a graphical temperature map corresponding to the tissue surface.

51. The system of claim 50, wherein the user interface is constructed and arranged to display the temperature map of at least one of a one-dimensional, two-dimensional, and three-dimensional representation of the tissue surface.

52. The system of claim 51, where the user interface is constructed and arranged to display the temperature map of a four-dimensional representation of the tissue surface.

53. The system of claim 50 wherein the user interface is constructed and arranged to display other temperature information.

54. The system of claim 53, wherein the other temperature information comprises at least one of peak temperature information, rate of change of temperature information, and average temperature information for multiple tissue surfaces.

55. A probe assembly, comprising:
a rotary motor having a rotatable hollow shaft extending along a longitudinal axis;
an optical device extending through the hollow shaft along the longitudinal axis;
a stationary fiber assembly in communication with the optical device;
a mounting sleeve coupled to the hollow shaft along the longitudinal axis; and
an optical element in a mounting sleeve, the optical element in direct communication with a distal end of the optical device for outputting received infrared energy to the distal end of the optical device, wherein the rotary motor rotates the hollow shaft relative to the fiber assembly along the longitudinal axis, and wherein the hollow shaft rotates the mounting sleeve about the longitudinal axis relative to the stationary fiber assembly.

56. The probe assembly of claim 55, further comprising a probe sheath about the rotary motor and mounting sleeve, the probe sheath include an infrared transmissive surface, wherein the optical element can receive the infrared energy from a tissue surface via the infrared transmissive surface.

57. The probe assembly of claim 55, wherein the optical device is a portion of the fiber assembly, and wherein the rotary motor rotates the hollow shaft about the fiber assembly.

58. The probe assembly of claim 57, further comprising a slip ring about at least a portion of the stationary fiber assembly, the slip ring positioned between the stationary fiber assembly and the hollow shaft.

59. The probe assembly of claim 58, wherein the slip ring is coupled to an exposed region of the hollow shaft at a proximal end of the rotary motor to align a combination of the optical element, the fiber assembly, and a stationary optical element adjacent a proximal end of the fiber assembly.

60. The probe assembly of claim 57, further comprising a separating element between the rotary motor and the mounting sleeve that surrounds an exposed region of the hollow shaft extending from the rotary motor.

61. The probe assembly of claim 60, wherein the separating element includes a lubricous material, bearing, or a running gap.

62. The probe assembly of claim 55, wherein the optical device includes an index-matched optical element between the fiber assembly and the optical element, and wherein the optical element directs infrared energy along the index-matched optical element to the fiber assembly.

63. The probe assembly of claim 55, further comprising an electrical connector for providing power to the rotary motor.

64. A temperature mapping system that produces temperature estimations of a tissue surface, comprising:
a probe assembly;
a fiber assembly extending through the probe assembly, the fiber assembly including at least one fiber constructed and arranged to receive infrared energy from the tissue surface;
a processor that converts the received infrared energy into temperature information signals; and
a motion unit coupled to the proximal end of the probe assembly, the motion unit constructed and arranged to at least one of rotate the at least one fiber about a longitudinal axis and translate the fiber assembly along the longitudinal axis at a speed that changes according to the temperature signals.

65. The system of claim 64, wherein the processor processes an amount of temperature data that is dependent on a rate of rotation and speed of translation of the fiber assembly by the motion unit.

66. The system of claim 64, wherein the motion unit increases a rotational speed of the fiber assembly when an area of interest at the tissue surface is identified.

67. The system of claim 65, wherein the motion unit decreases the translation speed of the fiber assembly and reduces a translation distance to the area of interest.

68. The system of claim 67, wherein the motion unit further increases the rotational speed of the fiber assembly.

69. The system of claim 65, wherein the motion unit proportionally increases the translation speed of the fiber assembly and increases the rate of rotation of the fiber assembly at or near the area of interest.

70. A system that produces temperature estimations of a tissue surface, comprising,
a monitoring unit that receives and displays the temperature information;
a probe assembly;
a fiber assembly extending through the probe assembly, the fiber assembly including at least one fiber constructed and arranged to receive infrared energy from the tissue surface;
a patient interface unit, comprising
a base;
a motion unit at the base, the motion unit constructed and arranged to at least one of rotate the at least one fiber relative to the base about the longitudinal axis and translate the at least one fiber relative to the base in
a linear direction along the longitudinal axis;
a first coupling mechanism coupled to the base; and
a second coupling mechanism at the motion unit, wherein the probe assembly is removably coupled to each of the first and second coupling mechanisms; and
a processor that converts the infrared energy received at the at least one fiber into a plurality of temperature measurements.

71. The system of claim 70, wherein the patient interface unit comprises a sensor assembly co-located with the rotary motor on the translation table.

72. A method of controlling a temperature measurement probe, comprising:
determining a first longitudinal position and a second longitudinal position of a distal end of a probe sheath, the first and second longitudinal positions spaced apart from each other in the longitudinal direction, a first region of interest being defined therebetween;
collecting, at a fiber extending through the probe sheath, data from tissue proximal the probe sheath in the first region of interest;
determining a second region of interest within the first region of interest, in response to the collected data; and
controlling a rate of movement of the fiber at a collection region to be different when collecting data within the second region of interest as compared to collecting data that lies within the first region of interest and beyond the second region of interest.

73. A system that produces temperature estimations of a tissue surface, comprising:
a base;
a probe assembly having a proximal end and a distal end, the proximal end of the probe assembly at the base and extending along a longitudinal axis, and including:
a handle at the proximal end of the probe assembly; and
a probe connector;
a fiber assembly extending through the probe assembly, the fiber assembly including at least one fiber constructed and arranged to receive infrared energy from the tissue surface;
a motion unit at the base, the motion unit constructed and arranged to at least one of rotate the at least one fiber relative to the base about the longitudinal axis and translate the at least one fiber relative to the base in a linear direction along the longitudinal axis;
a first coupling mechanism coupled to the base, wherein the handle is removably coupled to the first coupling mechanism; and
a second coupling mechanism at the motion unit, wherein the probe connector is removably coupled to the second coupling mechanism.

74. The system of at least one of the preceding claims, wherein the motion unit comprises:
a rotary motor having a hollow shaft, wherein the probe connector is positioned in the hollow shaft, and wherein the hollow shaft is driven by the motion unit to rotate the at least one fiber about the longitudinal axis; and
a linear motor that translates the at least one fiber and the rotary motor in a linear direction along the longitudinal axis.

75. The system of at least one of the preceding claims, wherein the rotary motor assembly and the linear motor operate independently of each other.

76. The system of at least one of the preceding claims, wherein the motion unit comprises:
a rotary motor having a hollow shaft, wherein the probe connector is positioned in the hollow shaft, and wherein the hollow shaft is driven by the motion unit to rotate the at least one fiber about the longitudinal axis.

77. The system of at least one of the preceding claims, wherein a proximal end of the probe connector includes a conical nose, wherein a proximal end of the at least one fiber is at the conical nose, and wherein a proximal end of the hollow shaft of the rotary motor mates with the conical nose of the probe connector.

78. The system of at least one of the preceding claims, further comprising an optical element adjacent the rotary motor, wherein the conical nose is positioned in the hollow shaft such that the at least one fiber is aligned with the optical element along the longitudinal axis.

79. The system of at least one of the preceding claims, wherein the conical nose of the probe connector is conformably positioned in a conical cavity of the hollow shaft of the rotary motor to maintain concentricity between the at least one fiber and the optical element during operation of the system.

80. The system of at least one of the preceding claims, wherein when the rotary motor rotates between two positions at a predetermined angle between the two positions, the at least one fiber rotates at the same predetermined angle and at the same time as the rotary motor.

81. The system of at least one of the preceding claims, wherein the second coupling mechanism includes a spring-biased rotary motor coupling at the hollow shaft of the rotary motor, the spring-biased rotary motor coupling having at least one groove, and wherein the probe connector includes at least one engagement pin constructed and arranged to mate with the at least one groove at the hollow shaft of the rotary motor.

82. The system of at least one of the preceding claims, further comprising an automatic coupling mechanism that couples the probe connector to the rotary motor by detecting the handle at the first coupling mechanism, and drives a connection interface of the rotary motor to the probe connector for interfacing with the probe connector.

83. The system of at least one of the preceding claims, wherein the rotary motor includes a plurality of counterweights coupled to the hollow shaft for providing a centripetal force, and wherein the second coupling mechanism is positioned at the counterweights for coupling to a proximal end of the probe connector.

84. The system of at least one of the preceding claims, wherein the second coupling mechanism comprises a collet and wherein the probe connector comprises a coupling that interfaces with the collet.

85. The system of at least one of the preceding claims, wherein the probe connector comprises at least one slot, the hollow shaft comprises at least one opening that aligns with the at least one slot of the probe connector, and wherein the system further comprises a linkage device that is positioned in the aligned at least one slot and opening to prevent the probe connector from moving axially with respect to the hollow shaft.

86. The system of at least one of the preceding claims, further comprising a control device that controls an insertion and removal of the linkage device with respect to the hollow shaft.

87. The system of at least one of the preceding claims, wherein the at least one probe connector slot include a ramp for applying a force in an axial direction for abutting the probe connector with an end of the hollow shaft.

88. The system of at least one of the preceding claims, wherein the hollow shaft of the rotary motor includes a threaded region, and wherein the probe connector comprises a thread that mates with the threaded region of the rotary motor.

89. The system of at least one of the preceding claims, further comprising a sensor at the first coupling mechanism that detects when the handle is coupled at the first coupling mechanism, and wherein the translation table moves the rotary motor in a direction relative to the probe connector for coupling the threaded probe connector with the threaded region of the rotary motor.

90. The system of at least one of the preceding claims, further comprising a linear motor that translates the at least one fiber in a linear direction along the longitudinal axis.

91. The system of at least one of the preceding claims, wherein the motion unit further comprises a translation table that is moved along the base by the linear motor in the linear direction along the longitudinal axis.

92. The system of at least one of the preceding claims, further comprising a locking mechanism coupled to the translation table, and an actuator coupled to the base, wherein the locking mechanism engages the actuator to prevent the translation table from a linear movement.

93. The system of at least one of the preceding claims, wherein the system is constructed and arranged to produce surface temperature estimations of a hollow body cavity having the tissue surface.

94. The system of at least one of the preceding claims, further comprising a sensor assembly having a sensor that receives the infrared energy from the at least one fiber, and converts the received infrared energy into temperature information signals.

95. The system of at least one of the preceding claims, wherein the sensor assembly is positioned on a positioning plate for aligning the sensor assembly with a proximal end of the at least one fiber.

96. The system of at least one of the preceding claims, wherein the positioning plate includes a positioning plate for adjusting the sensor assembly in at least one of a pitch, yaw, roll, x, y, and z direction relative to the proximal end of the at least one fiber.

97. The system of at least one of the preceding claims, wherein the sensor assembly comprises a cooling assembly constructed and arranged to cool one or more portions of the sensor.

98. The system of at least one of the preceding claims, further comprising a controller that processes the infrared energy received by the sensor assembly and generates an output that includes temperature data related to the processed infrared energy.

99. The system of at least one of the preceding claims, wherein a portion of the fiber assembly between the probe connector and the first coupling assembly extends in the linear direction along the longitudinal axis during translation of the at least one fiber.

100. The system of at least one of the preceding claims, wherein the at least one fiber extends directly between the first coupling assembly and the motion unit.

101. The system of at least one of the preceding claims, wherein the fiber assembly is passive, and is constructed and arranged to only collect infrared energy from the tissue surface.

102. The system of at least one of the preceding claims, wherein the first coupling mechanism includes a sheath bulkhead coupled to the base and having a slot for receiving the handle of the probe assembly.

103. The system of at least one of the preceding claims, wherein the sheath bulkhead includes a twist lock coupling at the slot, and wherein the handle includes a bayonet portion that mates with the twist lock coupling at the slot to prevent rotation of the handle about the longitudinal axis.

104. The system of at least one of the preceding claims, wherein the twist lock coupling includes a spring-loaded pin activation element and the bayonet portion of the handle includes at least one lobe, and wherein the spring-loaded pin activation element biases the at least one lobe at the sheath bulkhead unit.

105. The system of at least one of the preceding claims, wherein the motion unit comprises a Yankee screw and a rotary motor, wherein the Yankee screw includes a Yankee screw motor that translates the at least one fiber and the rotary motor in a linear direction along the longitudinal axis.

106. The system of at least one of the preceding claims, wherein the Yankee screw motor operates to rotate the Yankee screw, the Yankee screw including dual opposed continuous helical grooves and wherein the Yankee screw motor rotates the Yankee screw to translate the at least one fiber and the rotary motor in the linear direction.

107. The system of at least one of the preceding claims, wherein a translation speed and a rotational speed of the fiber assembly are both driven by the rotary motor.

108. The system of at least one of the preceding claims, wherein the at least one fiber collects infrared energy from a body lumen tissue surface while the rotary motor of the motion unit rotates the at least one fiber about the longitudinal axis.

109. The system of at least one of the preceding claims, wherein the at least one fiber collects infrared energy from a body lumen tissue surface while the motion unit at least one of translates the at least one fiber along the longitudinal axis and rotates the at least one fiber about the longitudinal axis.

110. The system of at least one of the preceding claims, further comprising a controller that processes infrared energy collected by the at least one fiber, and generates an output that includes temperature data related to the processed infrared energy.

111. The system of at least one of the preceding claims, wherein the output includes at least one of a two-dimensional (2D) graphical temperature map, a one-dimensional (1D) graphical temperature map, a temperature value, an alarm, and a temperature rate of change.

112. The system of at least one of the preceding claims, wherein the probe assembly further comprises a sheath coupled to the handle, wherein a distal end of the fiber is positioned in the sheath and at least one of translates and rotates relative to the sheath.

113. The system of at least one of the preceding claims, further comprising at least one marker band positioned at a distal end of the sheath, wherein the distal end of the fiber assembly is constructed and arranged to translate relative to the at least one marker band.

114. The system of at least one of the preceding claims, wherein the sheath includes an infrared opaque region at a distal side of the marker band, and an infrared transmissive region at a proximal side of the marker band.

115. The system of at least one of the preceding claims, wherein the at least one marker band comprises a distal band and a proximal band, and wherein the first fiber assembly is constructed and arranged to translate between the distal band and the proximal band.

116. The system of at least one of the preceding claims, wherein the translation assembly is constructed and arranged to translate the fiber in a reciprocating motion between the distal band and the proximal band, and wherein the fiber receives the infrared energy from a region between the distal band and the proximal band.

117. The system of at least one of the preceding claims, wherein the at least one marker band is constructed and arranged to cause a sensor in communication with a proximal end of the at least one fiber to produce a predetermined signal when the distal end of the at least one fiber receives infrared light from the at least one marker band.

118. The system of at least one of the preceding claims wherein the at least one marker band is C-shaped, and wherein the C-shaped marker band includes two ends, and a gap between the two ends.

119. The system of at least one of the preceding claims, wherein the gap identifies a rotational position of the at least one fiber.

120. The system of at least one of the preceding claims, wherein the gap provides a different and distinguishable signal from the rest of the marker band due to differences in emissivity between tissue and the marker band material.

121. The system of at least one of the preceding claims, further comprising a processor that converts the infrared energy received at the at least one fiber into a plurality of temperature measurements.

122. The system of at least one of the preceding claims, further comprising a display user interface that receives the temperature measurements from the processor, and displays a graphical temperature map corresponding to the tissue surface.

123. The system of at least one of the preceding claims, wherein the user interface is constructed and arranged to display the temperature map of at least one of a one-dimensional, two-dimensional, and three-dimensional representation of the tissue surface.

124. The system of at least one of the preceding claims, where the user interface is constructed and arranged to display the temperature map of a four-dimensional representation of the tissue surface.

125. The system of at least one of the preceding claims wherein the user interface is constructed and arranged to display other temperature information.

126. The system of at least one of the preceding claims, wherein the other temperature information comprises at least one of peak temperature information, rate of change of temperature information, and average temperature information for multiple tissue surfaces.

127. A probe assembly, comprising:
a rotary motor having a rotatable hollow shaft extending along a longitudinal axis;
an optical device extending through the hollow shaft along the longitudinal axis;
a stationary fiber assembly in communication with the optical device;
a mounting sleeve coupled to the hollow shaft along the longitudinal axis; and
an optical element in a mounting sleeve, the optical element in direct communication with a distal end of the optical device for outputting received infrared energy to the distal end of the optical device, wherein the rotary motor rotates the hollow shaft relative to the fiber assembly along the longitudinal axis, and wherein the hollow shaft rotates the mounting sleeve about the longitudinal axis relative to the stationary fiber assembly.

128. The probe assembly of at least one of the preceding claims, further comprising a probe sheath about the rotary motor and mounting sleeve, the probe sheath include an infrared transmissive surface, wherein the optical element can receive the infrared energy from a tissue surface via the infrared transmissive surface.

129. The probe assembly of at least one of the preceding claims, wherein the optical device is a portion of the fiber assembly, and wherein the rotary motor rotates the hollow shaft about the fiber assembly.

130. The probe assembly of at least one of the preceding claims, further comprising a slip ring about at least a portion of the stationary fiber assembly, the slip ring positioned between the stationary fiber assembly and the hollow shaft.

131. The probe assembly of at least one of the preceding claims, wherein the slip ring is coupled to an exposed region of the hollow shaft at a proximal end of the rotary motor to align a combination of the optical element, the fiber assembly, and a stationary optical element adjacent a proximal end of the fiber assembly.

132. The probe assembly of at least one of the preceding claims, further comprising a separating element between the rotary motor and the mounting sleeve that surrounds an exposed region of the hollow shaft extending from the rotary motor.

133. The probe assembly of at least one of the preceding claims, wherein the separating element includes a lubricous material, bearing, or a running gap.

134. The probe assembly of at least one of the preceding claims, wherein the optical device includes an index-matched optical element between the fiber assembly and the optical element, and wherein the optical element directs infrared energy along the index-matched optical element to the fiber assembly.

135. The probe assembly of at least one of the preceding claims, further comprising an electrical connector for providing power to the rotary motor.

136. A temperature mapping system that produces temperature estimations of a tissue surface, comprising:
a probe assembly;
a fiber assembly extending through the probe assembly, the fiber assembly including at least one fiber constructed and arranged to receive infrared energy from the tissue surface;
a processor that converts the received infrared energy into temperature information signals; and
a motion unit coupled to the proximal end of the probe assembly, the motion unit constructed and arranged to at least one of rotate the at least one fiber about a longitudinal axis and translate the fiber assembly along the longitudinal axis at a speed that changes according to the temperature signals.

137. The system of at least one of the preceding claims, wherein the processor processes an amount of temperature data that is dependent on a rate of rotation and speed of translation of the fiber assembly by the motion unit.

138. The system of at least one of the preceding claims, wherein the motion unit increases a rotational speed of the fiber assembly when an area of interest at the tissue surface is identified.

139. The system of at least one of the preceding claims, wherein the motion unit decreases the translation speed of the fiber assembly and reduces a translation distance to the area of interest.

140. The system of at least one of the preceding claims, wherein the motion unit further increases the rotational speed of the fiber assembly.

141. The system of at least one of the preceding claims, wherein the motion unit proportionally increases the translation speed of the fiber assembly and increases the rate of rotation of the fiber assembly at or near the area of interest.

142. A system that produces temperature estimations of a tissue surface, comprising,
a monitoring unit that receives and displays the temperature information;
a probe assembly;
a fiber assembly extending through the probe assembly, the fiber assembly including at least one fiber constructed and arranged to receive infrared energy from the tissue surface;
a patient interface unit, comprising
a base;
a motion unit at the base, the motion unit constructed and arranged to at least one of rotate the at least one fiber relative to the base about the longitudinal axis and translate the at least one fiber relative to the base in a linear direction along the longitudinal axis;
a first coupling mechanism coupled to the base; and
a second coupling mechanism at the motion unit, wherein the probe assembly is removably coupled to each of the first and second coupling mechanisms; and
a processor that converts the infrared energy received at the at least one fiber into a plurality of temperature measurements.

143. The system of at least one of the preceding claims, wherein the patient interface unit comprises a sensor assembly co-located with the rotary motor on the translation table.

144. A system for performing a medical procedure, comprising:
a base;
a probe assembly having a proximal end and a distal end, the proximal end of the probe assembly at the base and extending along a longitudinal axis, and including:
a handle at the proximal end of the probe assembly; and
a probe connector;
a fiber assembly extending through the probe assembly, the fiber assembly including at least one fiber constructed and arranged to receive infrared energy from the tissue surface;
a motion unit at the base, the motion unit constructed and arranged to at least one of rotate the at least one fiber relative to the base about the longitudinal axis and translate the at least one fiber relative to the base in a linear direction along the longitudinal axis;
a first coupling mechanism coupled to the base, wherein the handle is removably coupled to the first coupling mechanism; and
a second coupling mechanism at the motion unit, wherein the probe connector is removably coupled to the second coupling mechanism.

145. A system as described in reference to the figures.

146. A method of performing a medical procedure as described in reference to the figures.
